SEQUENCE LISTING (30 sequences)

SEQ ID NO: 1 - protein, 502 aa, Homo sapiens
MSLVLLSLAALCRSAVPREPTVQCGSETGPSPEWMLQHDLIPGDLRDLRVEPVTTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYKKKCVKAGSLWDPNITACKKNEETVEVNFTTTPLGNRYMALIQHSTIIGFSQVFEPHQKKQTRASVVIPVTGDSEGATVQLTPYFPTCGSDCIRHKGTVVLCPQTGVPFPLDNNKSKPGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 2 - protein, 505 aa, Pan troglodytes
MSLVLLSLAALCRSAVPREPTVQCGSETGPSPEWMLQHDLIPGDLRDLRVELVTTSVATGDYSILMNVSWVLRADASIRLLKATKICVTGKSNFQSYSCVRCNYTEAFQTQTRPSGGKWTFSYVGFPVELNTVYFIGAHNIPNANMNEDGPSMPVNFTSPGCLDHIMKYKKKCVKAGSLWDPNITACKKNEETVEVNFTTTPLGNRYMALIQHSTHSTVIGFSQVFEPHQKKQTRASVVIPVTGDSEGAMVQLTPYFPTCGSDCIRHKGIVVLCPQTGVPFPLDNNKSKLGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFRETDTKDDYNALSVCPKYHLMKDATAFCAELLHVKQQVSAGKRSQACHDGCCSL

SEQ ID NO: 3 - protein, 502 aa, Gorilla gorilla
MSLVLLSLAALCRSAVPREPTIQCGSETGPSPEWMLQHDLIPGDLRDLRVEPVKTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYTKKCVKAGSLWDPNITACKKNEETVEVNFTTTPLGNRYMALIQHSTIIGFSQVFEPHQKKQTRASVVIPVTGDSEGAMVQLTPYFPTCGSDCIRHKGTVVLCPQTGVPFPLDNNKSKPGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFRETDTKDDYNALSVCPKYHLMKDATAFCAELLHVKQQVSAGKRSQACHDGCCSL

SEQ ID NO: 4 - protein, 454 aa, Pteropus alecto
MGGDDLAMSLMLLSLAALCWGAVSPEPTIQCGPETGPSPEWMVRHTLTPGDLRDLRVEPVKSSVASEDYSILMNISWILRADASIRLLKATKICVTGKSGFQSYGCVRCNYTEVFQTQSRPSGGKWMFFYIGFPVELNTLYFIGAHNIPNANMNEDSPSMSVNFTSPGSLWDPNITACKKNENMVEVNFTISPLGNRYMVLILKNTVIGTSVSEEKLTRTSVMVPVTGESEGAVVQLTPYFHTCGNDCIRRKGMVVLCPQTGVPFSPDNKKIKMSLSSTMLLPITVLVVYPSEICFHHTVCYFAEFLQNHCRSQVILDKWQKKKIAEMGPVQWLTTQKKAADKVIFLLSNDVNTICDGTCGKSEGSPCENSQDLFPLAFNLFCSDLRSQTHQHKYIVVYFREADTKDDYNALNVCPKYCLMKDATSFCMELLHVEQQVSTGKRLRACHNRCSSL

SEQ ID NO: 5 - protein, 354 aa, Rattus norvegicus
MNEDSPSLSVNFTSPGCLNHVMKYKKQCIEAGSLWDPNITACKKNEKTVEVNFTTNSLGNRYMVLIRRDTMLGVSIVLENKLTRTSVVIPVNDESEGALVELTPYLHTCDNDCIRRKGTVVLCSETSAPFPPDDNRSMLRGWLPLLLVLLVATWVLAVGIYLTWRQGRSTKTSFPITAMLLPLVKVLVVYPSEICFHHTVCRFTDFLQNYCRSEVILEKWQKKKIAEMGPVQWLTTQKQAADKVVFLLPSDAPSLCDSACGHKEGSATENSQDLFPLAFNLFCSDFSSQTHLHKYLVVYLGGADLKGDYNALRVCPQYHLMKDAPAFHTELLKATQSMPLKKRPQACHGSCSPL

SEQ ID NO: 6 - protein, 499 aa, Mus musculus
MLLVLLILAASCRSALPREPTIQCGSETGPSPEWMVQHTLTPGDLRDLQVELVKTSVAAEEFSILMNISWILRADASIRLLKATKICVSGKNNMNSYSCVRCNYTEAFQSQTRPSGGKWTFSYVGFPVELSTLYLISAHNIPNANMNEDSPSLSVNFTSPGCLNHVMKYKKQCTEAGSLWDPDITACKKNEKMVEVNFTTNPLGNRYTILIQRDTTLGFSRVLENKLMRTSVAIPVTEESEGAVVQLTPYLHTCGNDCIRREGTVVLCSETSAPIPPDDNRRMLGGWLPLFLVLLVAVWVLAAGIYLTWRQGRSTKTSFPISTMLLPLIKVLVVYPSEICFHHTVCRFTDFLQNYCRSEVILEKWQKKKIAEMGPVQWLTTQKQAADKVVFLLPSDVPTLCDSACGHNEGSARENSQDLFPLAFNLFCSDFSSQTHLHKYLVVYLGGADLKGDYNALSVCPQYHLMKDATAFHTELLKATQSMSVKKRSQACHDSCSPL

SEQ ID NO: 7 - protein, 499 aa, Equus caballus
MSLVLLSLAALCWGAVPREPTIQCGSEAGPSPEWMVQHALTPGDLRDLQVEPVKSRVATEDYSVLMNISWILRADASIRFLKATKICVTGKSNFQSYSCVRCNYTEAFQTQTRPSGGKWTFSYIGFPVELDTLYFIGAHNIPNANMNGDGPSLSVNFTSPGCLDHVMKYKKKCIEAGSLWDPNITACKKNEKMVEVNFTTSPLGNRYMALIQNNTVIGLSNVLENKLTRTSVVIPVTGESEGAVVQLTPYFHTCGSDCIRRRGIVVLCPHTGASSPPDNSRSVLGGWLPFLLPALLVATWVLAVGIYLTWRHERIKKTSFPTTALLSPIKVLVVYPSEICFHHTVCYFTKFLQNHCRTEVILEKWQKKKIAEMGPVQWLTTQKKAADKVIFLLSNNVNNVCDGTCGKSEGSPHENSQDLFPLAFNLFCSDLRSQNHLHKYMVVYFREADTKDDYDALHVCPKYCLMKDAAAFCTELLHVEQHVSVGKRWRACHNRCSAL

SEQ ID NO: 8 - protein, 477 aa, Sus scrofa
MLLVLLSLAALCWGAMPPEPTIQCGSEPGLSPEWMVRHALTPGDLRDLRVEPIKSSVAVEDYSILMNISWILRADASIRLLKATKICVTGKSQKQTYSCVRCNYTEAFQTQTRPSGGKWMFSYVGFPVELNTRYFIGAHNIPNANMNEDGPSLAVNFTSPGCLDRIMKYKKKCIEAGSLWDPNITACKKSENTVEVNFTTSPLGNRYMALIQNSTVIGTSYVSELTPYFRTCGNDCIRRRGTVVRCPHTGVPFPQDQSRSMLSGWLPLLLLALLVAIWVLAGGIYLTRRHERIKKTSFSATILLPPIKVLVVYPSEICFHHTVCYFTRFLQNHCRSEVILEKWQKKKIAEMGPVQWLTTQKAAADKVIFLLSNDGNTACDGTCSNSEGGPHENSQDLFPLAFNLFCSDLRSQTHLHKYVVVYFREVDIKDDYSALSVCPTYHLMKDAPAFCKELLHAEQHVSVGRRLQACHYSCSSL

SEQ ID NO: 9 - protein, 494 aa, Felis catus
MSLALLSLAALCWGLVSTEPTIQCGSEPGPSPEWMVQHTLTPGDLRDLRVEPVRSRVAMDYSILMNVSWVLRADASIRLLKATKICVTGKSNLQSYSCVRCNYTEAFQTQTRPSGGRWTFSYVGFPVELNTVYFIGAHNIPNANINEDSPSMSVNFTSPGCLDHIMKYQKKCIKAGSLWDPNVTACKKNKTVVEVNFTTSPLGNKYMALIQNRIVIGFSNVLENKPPTRTSVVIPVTGESEGAMVQLTPYFHTCGNDCIRRKGTVVLCPQTGISFPLDGRRSMMGGWQPFLLPALLGATALLAAGIYVIWRHKRIKKASFPTATLLSPIKVLVVYPSEICFHHTVCHFTEFLQNHCRSEVILEEWQKKKIAEMGPVQWLTTQKKAVDKIIFLLSNDVNTMCDSTCDKSEGSPHENSQDLFPLAFNLFCSDLRNQTPLRKYMVVYFREADTKDEYSALSVCPKYRLMKDAPAFCTELLRVEQHMSAGKRLTACSM

SEQ ID NO: 10 - protein, 14 aa, artificial sequence
  IL-RB inhibitory petpide motif 1
  MISC_FEATURE (1)..(1): Xaa can be valine (V), isoleucine (I), leucine (L), alanine (A), methionine (M)
  MISC_FEATURE (4)..(4): Xaa can be glycine (G) or serine (S)
  MISC_FEATURE (5)..(5): Xaa can be threonine (T) or alanine (A)
  MISC_FEATURE (7)..(7): Xaa can be glycine (G), serine (S) or aspartic acid (D)
  MISC_FEATURE (8)..(8): C=Xaa can be lysine (K), histidine (H) or asparagine (N)
  MISC_FEATURE (9)..(9): Xaa can be serine (S), lysine (K) or asparagine (N)
  MISC_FEATURE (12)..(12): Xaa can be serine (S) or glycine (G)
  MISC_FEATURE (13)..(13): Xaa can be proline (P) or alanine (A)
  MISC_FEATURE (14)..(14): Xaa can be serine (S), cysteine (C), Threonine (T), arginine (R) or histidine (H)
XCDXXCXXXEGXXX

SEQ ID NO: 11 - protein, 14 aa, Artificial Sequence
  IL-17RB inhibitory peptide motif 2
  MISC_FEATURE (1)..(1): Xaa can be valine (V), isoleucine (I) or leucine (L)
  MISC_FEATURE (4)..(4): Xaa can be glycine (G) or serine (S)
  MISC_FEATURE (5)..(5): Xaa can be threonine (T) or alanine (A)
  MISC_FEATURE (8)..(8): Xaa can be lysine (K) or histidine (H)
  MISC_FEATURE (9)..(9): Xaa can be serine (S), lysine (K) or asparagine (N)
  MISC_FEATURE (13)..(13): Xaa can be proline (P) or alanine (A)
  MISC_FEATURE (14)..(14): Xaa can be serine (S), cysteine (C), Threonine (T), arginine (R) or histidine (H)
XCDXXCGXXEGSXX

SEQ ID NO: 12 - protein, 14 aa, Artificial Sequence
  IL-17RB inhibitory peptide motif 3
  MISC_FEATURE (1)..(1): Xaa can be valine (V) or isoleucine (I)
  MISC_FEATURE (14)..(14): Xaa can be serine (S), cysteine (C) or histidine (H)
XCDGTCGKSEGSPX

SEQ ID NO: 13 - protein, 14 aa, Artificial Sequence
  IL-17RB inhibitory peptide motif 4
  MISC_FEATURE (14)..(14): Xaa can be serine (S) or histidine (H)
VCDGTCGKSEGSPX

SEQ ID NO: 14 - protein, 14 aa, Homo sapiens
VCDGTCGKSEGSPS

SEQ ID NO: 15 - protein, 14 aa, Pteropus alecto
ICDGTCGKSEGSPC

SEQ ID NO: 16 - protein, 14 aa, Rattus norvegicus
LCDSACGHKEGSAT

SEQ ID NO: 17 - protein, 14 aa, Mus musculus
LCDSACGHNEGSAR

SEQ ID NO: 18 - protein, 14 aa, Equus caballus
VCDGTCGKSEGSPH

SEQ ID NO: 19 - protein, 14 aa, Sus scrofa
ACDGTCSNSEGGPH

SEQ ID NO: 20 - protein, 14 aa, Felis catus
MCDSTCDKSEGSPH

SEQ ID NO: 21 - protein, 9 aa, Human immunodeficiency virus
PKKRRQRRR

SEQ ID NO: 22 - protein, 16 aa, Antennapedia homeodomain
RQIKIWFQNRRMKWKK

SEQ ID NO: 23 - protein, 18 aa, Zea mays
VRLPPPVRLPPPVRLPPP

SEQ ID NO: 24 - protein, 17 aa, Human immunodeficiency virus type 1
TRQARRNRRRRWRERQR

SEQ ID NO: 25 - protein, 15 aa, Flock house virus
RRRRNRTRRNRRRVR

SEQ ID NO: 26 - protein, 13 aa, human T-cell leukemia viruse
TRRQRTRRARRNR

SEQ ID NO: 27 - protein, 16 aa, SIMIAN VIRUS 40
KRPAAIKKAGQAKKKK

SEQ ID NO: 28 - protein, 27 aa, Homo sapiens
GWTLNSAGYLLGKINLKALAALAKKIL

SEQ ID NO: 29 - protein, 18 aa, Mus musculus
LLIILRRRIRKQAHAHSK

SEQ ID NO: 30 - protein, 23 aa, Artificial Sequence
  IL-17RB inhibitory peptide, fusion protein
RKKRRQRRRVCDGTCGKSEGSPS

(19) United States
(12) Patent Application Publication
(10) Pub. No.: US 2024/0124528 A1
(43) Pub. Date: Apr. 18, 2024

(54) ANTAGONIST OF INTERLEUKIN-17B RECEPTOR (IL-17RB) AND USE THEREOF

(71) Applicant: Academia Sinica, Taipei City (TW)

(72) Inventors: Wen-Hwa LEE, Taipei (TW); Heng-Hsiung WU, Taichung City (TW); Chun-Mei HU, Taipei (TW); Chun-Kai HUANG, Taipei City (TW)

(73) Assignee: Academia Sinica, Taipei City (TW)

(21) Appl. No.: 18/266,882

(22) PCT Filed: Dec. 14, 2021
(86) PCT No.: PCT/US2021/063226

§ 371 Date: Jun. 13, 2023

Related U.S. Application Data
(60) Provisional application No. 63/125,148, filed on Dec. 14, 2020.

(51) Int. Cl. C07K 14/00; A61P 35/00; G01N 33/574; G01N 33/68

(52) U.S. Cl. CPC C07K 14/001; A61P 35/00; G01N 33/57488; G01N 33/6869; A61K 38/00

(57) ABSTRACT

The present invention relates to an antagonist of interleukin-17B receptor (IL-17RB) which features interruption of the interaction of IL-17RB and MLK4. The present invention also relates to use of such antagonist for treatment of diseases or disorders associated with IL-17RB activation. Further disclosed is a phosphorylated IL-17RB as a biomarker for predicting prognosis and/or monitoring progression of cancer.

### RELATED APPLICATIONS

This application claims the benefit of U.S. provisional application No. 63/125,148, filed Dec. 14, 2020 under 35 U.S.C. § 119, the entire content of which is incorporated herein by reference.

### TECHNOLOGY FIELD

The present invention relates to an antagonist of interleukin-17B receptor (IL-17RB) which features interruption of the interaction of IL-17RB and MLK4. The present invention also relates to use of the antagonist for treatment of diseases or disorders associated with IL-17RB activation. Further disclosed is a phosphorylated IL-17RB useful as a biomarker for predicting prognosis and/or monitoring progression of cancer.

### BACKGROUND OF THE INVENTION

The interleukin-17 (IL-17) family consists of at least six ligands (A to F) with 20-50% sequence homology, and five cognate receptors (RA to RE) (1). IL-17A, -C, -E, and -F mainly play roles in mediating inflammation in autoimmune, allergic, and chronic inflammatory diseases, while the roles of IL-17B and IL-17D are less clear in pro-inflammatory function. The IL-17 receptors (IL-17R) are single-pass transmembrane proteins with conserved structural features (2). Specifically, they contain two extracellular fibronectin II-like domains and a cytoplasmic SEFIR domain, which has a role in triggering downstream signaling (3). IL-17RA mediates signaling through hetero-dimerization with IL-17RC for IL-17A and IL-17F, with IL-17RB for IL-17E, with IL-17RE for IL-17C (4), and with IL-17RD for IL-17A (5). However, whether IL-17B binds to IL-17RB homodimers or heterodimers (6, 7) remains unclear.

In spite of IL-17 receptor family similarity, each receptor has its own distinct structural characteristics. Through detailed genetic analysis, IL-17RA contains approximately 100 additional residues beyond the SEFIR domain, termed the SEFEX domain, which is also required for signaling (8, 9). Based on subsequent X-ray crystallographic studies, both domains form a single composite structural motif (10). Moreover, the cytoplasmic tail of IL-17RA contains a distinct domain, termed the C/EBP-b activation domain (CBAD), which binds to TNF receptor-associated factor 3 (TRAF3) and the ubiquitin-editing enzyme A20 (11-13). However, unlike IL-17RA, the SEFIR region of IL-17RB exhibits a very different 3D topology (10, 14), and its C-terminal lacks the CBAD domain, suggesting that IL-17RB may operate in a distinct manner.

Cancer cells are known to exploit various signaling pathways responsible for cell proliferation, division, differentiation and migration to gain a growth advantage. Pro-inflammatory cytokines are involved in tumor progression through modulation of the inflammatory tumor microenvironment (15). Nevertheless, driving cancer cell progression through pro-inflammatory cytokine pathways is rarely found. Intriguingly, overexpression of IL-17RB in pancreatic cancer (7), breast cancer (6, 16, 17), and other neoplasms correlates with their malignancy. Depletion of IL-17RB or treatment with neutralizing antibodies against IL-17RB abolished tumor growth and metastasis (7), suggesting the importance of this pro-inflammatory receptor in these cancers. This potential oncogenic function of IL-17RB is similar to the well-recognized receptor tyrosine kinases (RTKs), which play a critical role in oncogenic processes in many cancers. All RTKs share a similar protein structure comprised of an extracellular ligand binding domain, a single transmembrane helix, and a cytoplasmic region that contains a tyrosine kinase domain (TKD) and a carboxyl (C)-terminal tail (18). RTKs are generally activated by receptor-specific ligands by binding to extracellular regions of RTKs, and the receptor is activated by ligand-induced receptor dimerization and/or oligomerization (18). For most RTKs, this conformational change allows the TKD to assume an active conformation for autophosphorylation of RTKs and engages downstream mediators that propagate critical cellular signaling pathways. However, IL-17RB lacks a defined kinase domain and is not an RTK. How IL-17RB responds to its ligand and transmits the signal to downstream mediators for its oncogenic function remains enigmatic.

### SUMMARY OF THE INVENTION

In this present invention, it is unexpectedly found that IL-17RB forms a homodimer upon IL-17B binding, and recruits MLK4, a dual kinase, to phosphorylate it at tyrosine 447. The tyrosine-phosphorylated IL-17RB, in turn, recruits TRIM56, a ubiquitin ligase, to add K63-linked ubiquitin chains on lysine 470. Introduced mutations of Y447F or K470R in IL-17RB fail to transmit oncogenic signaling. The significance of this signaling mechanism in cancer is further demonstrated by blocking mixed-lineage kinase 4 (MLK4, also known as KIAA1804 and MAP3K21) binding to IL-17RB with a specific peptide containing amino acid sequence 403-416 of IL-17RB, leading to a loss of Y447 phosphorylation and K470 ubiquitination, thereby reducing tumorigenesis and metastasis and prolonging the lifespan of pancreatic tumor-bearing mice. It is also found that the MLK4-mediated IL-17B/IL-17RB oncogenic signaling is independent and distinct from IL-17E/IL-17RB-mediated immunogenic signaling and therefore inhibition of the MLK4-mediated IL-17B/IL-17RB oncogenic signaling is clinically beneficial for treating a relevant proliferative disorder without side effects resulted from blockage of IL-17E induced IL-17RB immunogenic signaling.

Therefore, in one aspect, the present invention provides a method for inhibiting IL-17B/IL-17RB activation and/or treating a disease or disorder associated with IL-17B/IL-17RB activation (such as a proliferative disorder e.g. a cancer or its metastasis) by administering to a subject in need an effective amount of an IL-17RB antagonist that targets the interaction between IL-17RB and MLK4, and/or Y447 phosphorylation, and/or K470 ubiquitination of IL-17RB. In some instances, the IL-17RB antagonist is a peptide or a small molecule inhibiting the binding of MLK4 to IL-17RB. Specifically, the IL-17RB antagonist does not involve IL-17E/IL-17RB signaling and thus does not inhibit IL-17E/IL-17RB-mediated type 2 immunity in the subject.

In some embodiments, the IL-17RB antagonist is an IL-17RB inhibitory peptide comprising a first segment that comprises the amino acid sequence X₁CDX₂X₃CX₄X₅X₆EGX₇X₈X₉ (SEQ ID NO: 10), wherein X₁ is valine (V), isoleucine (I), leucine (L), alanine (A), methionine (M), X₂ is glycine (G) or serine (S), X₃ is threonine (T) or alanine (A), X₄ is glycine (G), serine (S) or aspartic acid (D), X₅ is lysine (K), histidine (H) or asparagine (N), X₆ is serine (S), lysine (K) or asparagine (N), X₇ is serine (S) or glycine (G), X₈ is proline (P) or alanine (A), and X₉ is serine (S), cysteine (C), Threonine (T), arginine (R) or histidine (H).

In some embodiments, the first segment comprises the motif of X₁CDX₂X₃CGX₅X₆EGSX₈X₉ (SEQ ID NO: 11), wherein X₁ is valine (V), isoleucine (I) or leucine (L), X₂ is glycine (G) or serine (S), X₃ is threonine (T) or alanine (A), X₅ is lysine (K) or histidine (H), X₆ is serine (S), lysine (K) or asparagine (N), X₈ is proline (P) or alanine (A), and X₉ is serine (S), cysteine (C), Threonine (T), arginine (R) or histidine (H).

In some embodiments, the first segment comprises the motif of X₁CDGTCGKSEGSPX₉ (SEQ ID NO: 12), wherein X₁ is valine (V) or isoleucine (I), and X₉ is serine (S), cysteine (C) or histidine (H).

In some embodiments, the first segment comprises the motif of VCDGTCGKSEGSPX₉ (SEQ ID NO: 13), wherein X₉ is serine (S) or histidine (H).

In some embodiments, the first segment comprises the amino acid sequence selected from the group consisting of VCDGTCGKSEGSPS (SEQ ID NO: 14, human), VCDGTCGKSEGSPS (SEQ ID NO: 14, chimpanzee), VCDGTCGKSEGSPS (SEQ ID NO: 14, gorilla), ICDGTCGKSEGSPC (SEQ ID NO: 15, fox), LCDSACGHKEGSAT (SEQ ID NO: 16), LCDSACGHNEGSAR (SEQ IDNO: 17, mouse), VCDGTCGKSEGSPH (SEQ ID NO: 18, horse), ACDGTCSNSEGGPH (SEQ ID NO: 19), and MCDSTCDKSEGSPH (SEQ ID NO: 20, cat).

In some embodiments, the first segment comprises the amino acid sequence selected from the group consisting of SEQ ID NO: 14-18.

In some embodiments, the first segment comprises the amino acid sequence selected from the group consisting of SEQ ID NO: 14, 15 and 18.

In some embodiments, the first segment comprises the amino acid sequence selected from the group consisting of SEQ ID NO: 14 and 18.

In some embodiments, the first segment is fused to a second segment that comprises a cell-penetrating peptide sequence.

In some embodiments, the cell-penetrating peptide sequence is selected from the group consisting of

(SEQ ID NO: 21)
RKKRRQRRR
(HIV-TAT)
(SEQ ID NO: 22)
RQIKIWFQNRRMKWKK
(Penetratin)
(SEQ ID NO: 23)
VRLPPPVRLPPPVRLPPP
(SAP)
(SEQ ID NO: 24)
TRQARRNRRRWRERQR
(HIV-1 Rev)
(SEQ ID NO: 25)
RRRNRTRRNRRRVR
(FHV)
(SEQ ID NO: 26)
TRRQRTRRARRNR
(HTLV-II)
(SEQ ID NO: 27)
KRPAAIKKAGQAKKKK
(NLS)
(SEQ ID NO: 28)
GWTLNSAGYLLGKINLKALAALAKKIL
(Transportan)
(SEQ ID NO: 29)
LLIILRRRIRKQAHAHSK
(pVEC).

In one certain example, the IL-17RB inhibitory peptide comprises or consists of the amino acid sequence as set forth in RKKRRQRRRVCDGTCGKSEGSPS SEQ ID NO: 30.

In some embodiments, the peptide is a loop (cyclic) peptide.

In some embodiments, the IL-17RB inhibitory peptide has a length of less than 100 amino acids, e.g. 80 amino acids or less, 60 amino acids or less, 40 amino acids or less, or 30 amino acids or less.

In another aspect, the present invention provides an IL-17RB inhibitory peptide that inhibits the binding of MLK4 to IL-17RB as described herein.

In a further aspect, the present disclosure provides a recombinant nucleic acid comprising a nucleotide sequence encoding any of the peptides as described herein. Such a nucleic acid may be a vector comprising the coding sequence noted herein. In some examples, the vector is an expression vector.

Any of the peptides or nucleic acids may be formulated to form a composition, which further comprises a physiologically acceptable carrier. In some instance, the composition of the present invention is a pharmaceutical composition for medical use.

According to the present invention, any of the IL-17RB inhibitory peptides or an encoding nucleic acid thereof or a composition comprising the peptide or the encoding nucleic acid is useful in inhibiting IL-17B/IL-17RB activation and/or treating a disease or disorder associated with such activation in a subject in need thereof. Typically, the disease or disorder is IL-17B/IL-17RB-mediated proliferation disorder e.g. a cancer and a metastasis thereof.

In some embodiments, the cancer is selected from the group consisting of lung cancer, pancreatic cancer, breast cancer, colorectal cancer, liver cancer, kidney cancer, head and neck cancer, esophageal cancer, gastric cancer, biliary tract cancer, gallbladder and bile duct cancer, mammary cancer, ovarian cancer, cervical cancer, uterine body cancer, bladder cancer, prostate cancer, testicular tumor, osteogenic and soft-tissue sarcomas, leukemia, malignant lymphoma, multiple myeloma, skin cancer, brain tumor and plural malignant mesothelioma.

In one certain example, the cancer is breast cancer.

In another certain example, the cancer is pancreatic cancer.

It is also found in this invention that the phosphorylated IL-17RB (particularly P-Y447) correlates with negative (poor) prognosis of cancer. Therefore, the present invention provides a method for predicting the prognosis of cancer comprising collecting a biological sample obtained from a cancerous patient, measuring the expression level of phosphorylated IL-17RB in the sample, and determining the prognosis of the cancer in the patient based on the expression level of phosphorylated IL-17RB in the sample, wherein an elevated level of phosphorylated IL-17RB in the sample indicates poor prognosis. The present invention also provides a method for monitoring progression of cancer in a cancer patient, comprising (a) measuring a level of phosphorylated IL-17RB protein in a first biological sample obtained from the patient at a first time-point; (b) measuring a level of phosphorylated IL-17RB protein in a second biological sample obtained from the patient at a second time-point; and (c) determining cancer progression in the patient based on the levels in the first and second biological samples wherein an elevated level of phosphorylated IL-17RB protein in the second biological sample as compared to that in the first biological sample is indicative of cancer progression. In one instance, the cancer is pancreatic cancer.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1A to 1H. Tyrosine 447 (Y447) in the intracellular domain of IL-17RB is critical for IL-17RB oncogenic signaling. (FIG. 1A) Immunoblots of phosphorylation at tyrosine, serine and threonine residues of IL-17RB immunoprecipitated by anti-IL-17RB antibody (D9) from the CFPAC1 cells treated with rIL-17B. (FIG. 1B) Sequence of the IL-17RB intracellular domain with the six tyrosine residues. The amino acid sequence of the full-length human IL-17RB is SEQ ID NO: 1. (FIG. 1C) Immunoblotting analysis of IL-17RB-KO BxPC3 cells expressing the wild-type (WT) and six tyrosine (Y)-to-phenylalanine (F) mutants of IL-17RB. The IL-17RB-KO BxPC3 cells were treated with rIL-17B and the cell lysates were immunoprecipitated with anti-IL-17RB or control mIgG (mouse IgG) followed immunoblotting with the indicated antibodies or directly immunoblotted with the indicated antibodies as the input control. (FIG. 1D) The P-Y447 antibody recognizes WT, but not Y447F, IL-17RB after IL-17B treatment. IL-17RB-KO BxPC3 cells expressing Flag-tagged WT and Y447F IL-17RB were treated with rIL-17B and the cell lysates were immunoprecipitated by anti-Flag-conjugated beads. The blot was probed by P-Y447-specific antibody or directly immunoblotted with the antibodies indicated as the input control. (FIG. 1E) Immunoblotting analysis of the expression of P-Y447 of IL-17RB depending on the amount of IL17B. BxPC3 cells were treated with the indicated amounts of rIL-17B and the cell lysates were immunoblotted with the indicated antibodies. (FIG. 1F to FIG. 1H) Y447 is essential for IL-17RB oncogenic signaling. (FIG. 1F) Immunoblotting analysis of CFPAC1 cells expressing IL-17RB and the six mutants as in (FIG. 1C) with the indicated antibodies. (FIG. 1G) Relative mRNA expression of CCL20, CXCL1 and TFF1 in the same cells were measured by RT-qPCR (n=3). (FIG. 1H) SACF assay of the same cells (n=6).

FIGS. 2A to 2C include charts showing that high expression of IL-17RB P-Y447 parallels with high IL-17RB amounts and correlates with worse progression of patients with pancreatic cancer. (FIG. 2A) Representative IHC images of three serial sections of a BxPC3 xenograft tumor staining with anti-P-Y477 or peptide-pre-absorbed anti-sera to verify the antibody specificity. Scale bars: 50 μm. (FIG. 2B) Overall survival of the 87 patients with pancreatic cancer with different levels of P-Y447 expression was plotted with the Kaplan-Meier method. Log-rank test was used. (FIG. 2C) The plot showed the proportion of post-operative progression within one year of 44 patients with pancreatic cancer with high or low levels of P-Y447 expression. The dashed line indicates the median time of cancer progression. #Progression# was defined by post-operative recurrence and/or metastasis. Log-rank test was used.

FIGS. 3A to 3G include charts showing identification of MLK4 critical for IL-17B/IL-17RB oncogenic signaling in pancreatic cancer cells. (FIG. 3A) Flow chart described the process for identifying IL-17RB-interacting proteins (left).

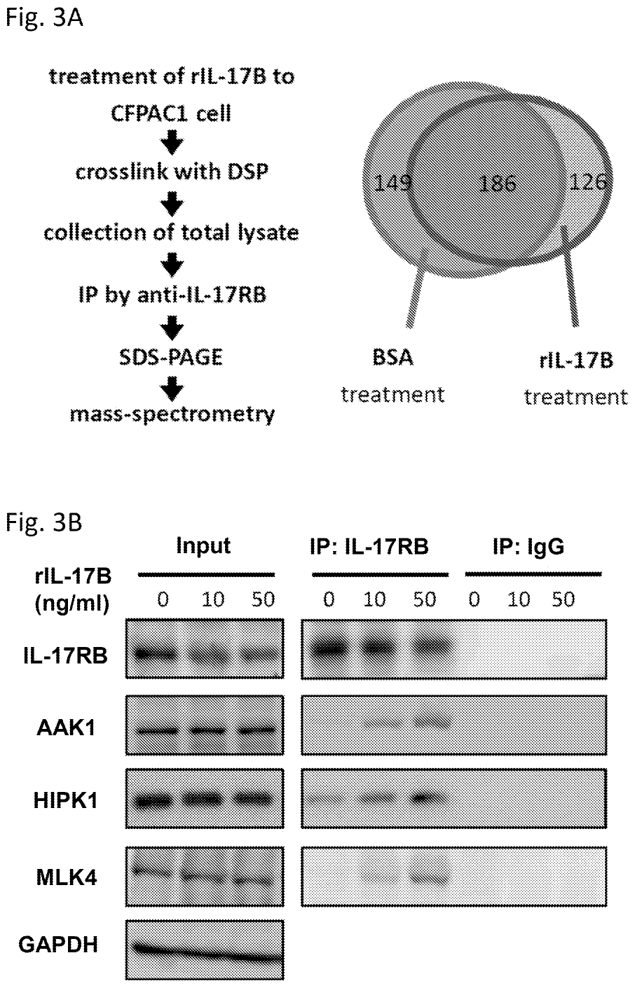

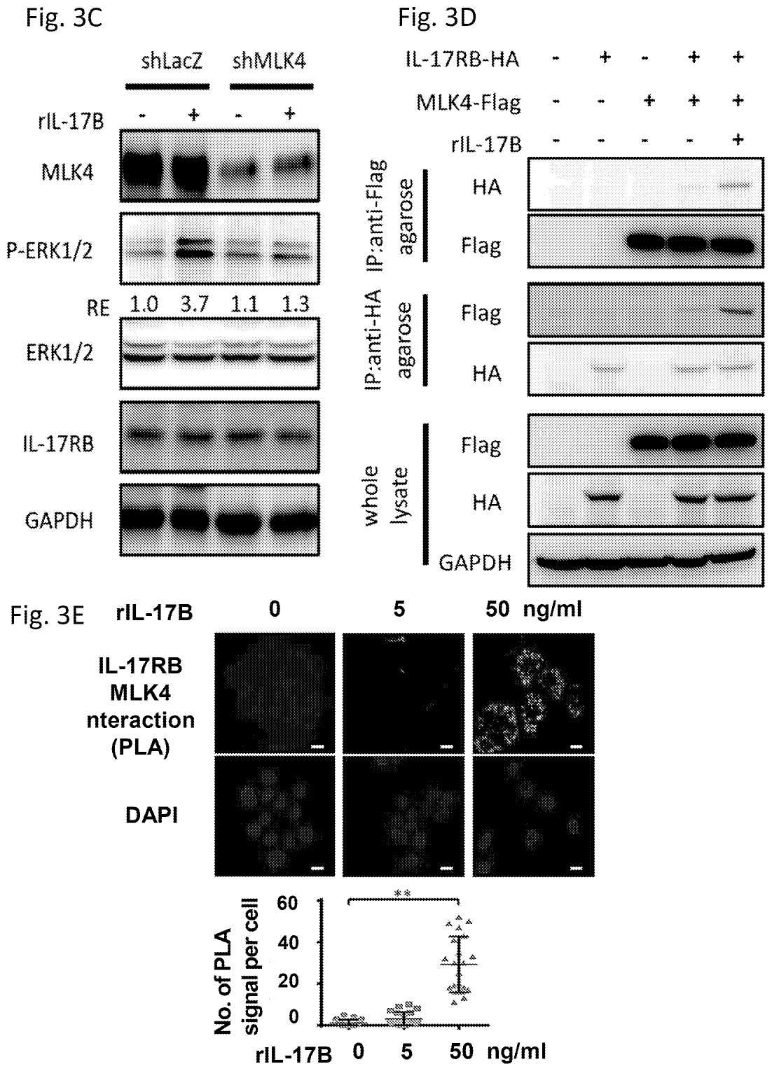

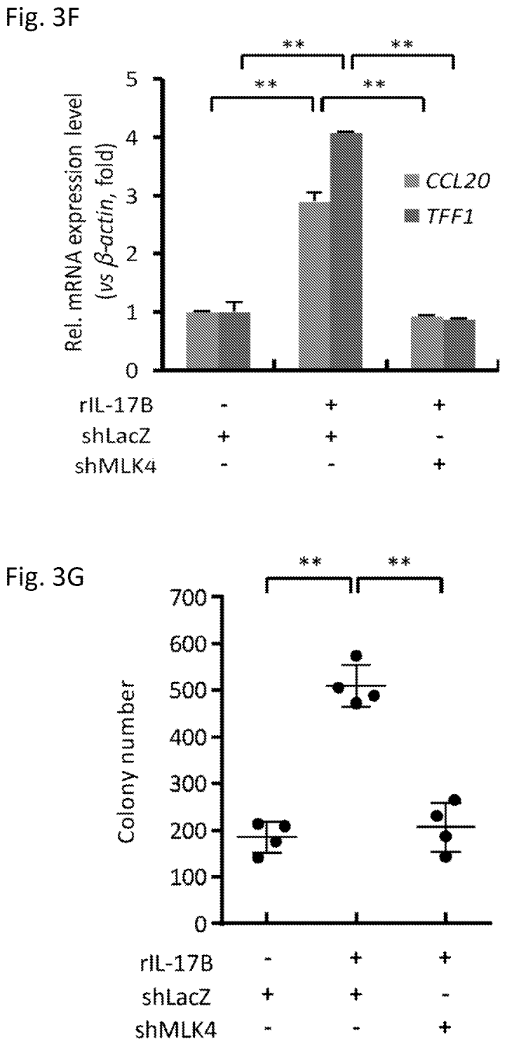

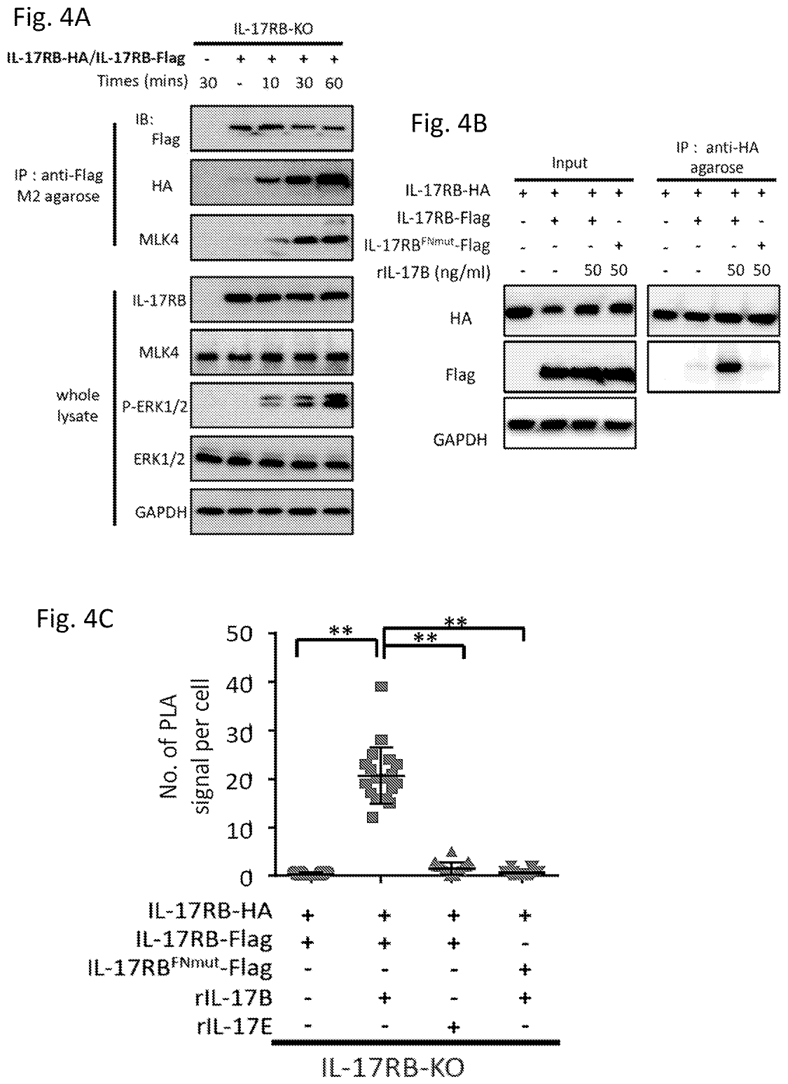

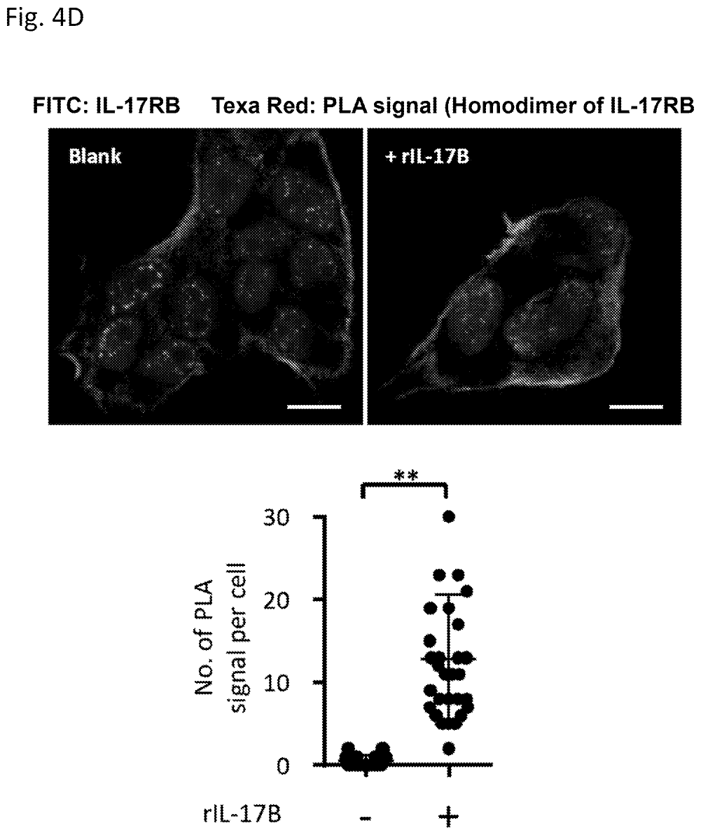

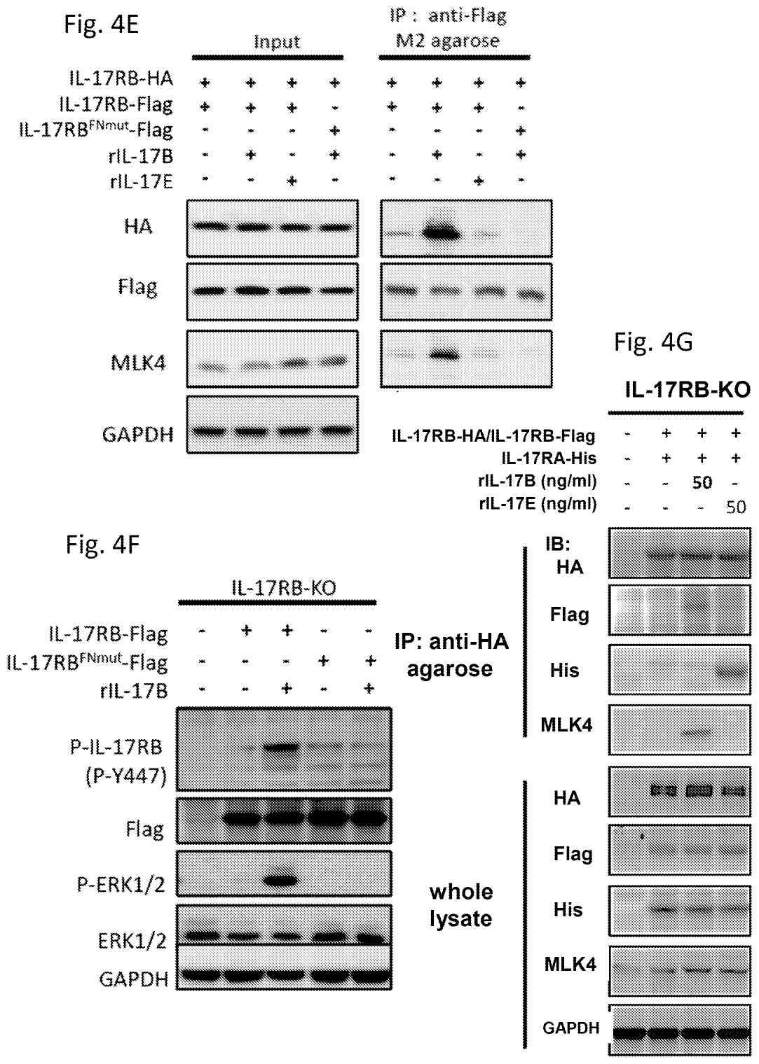

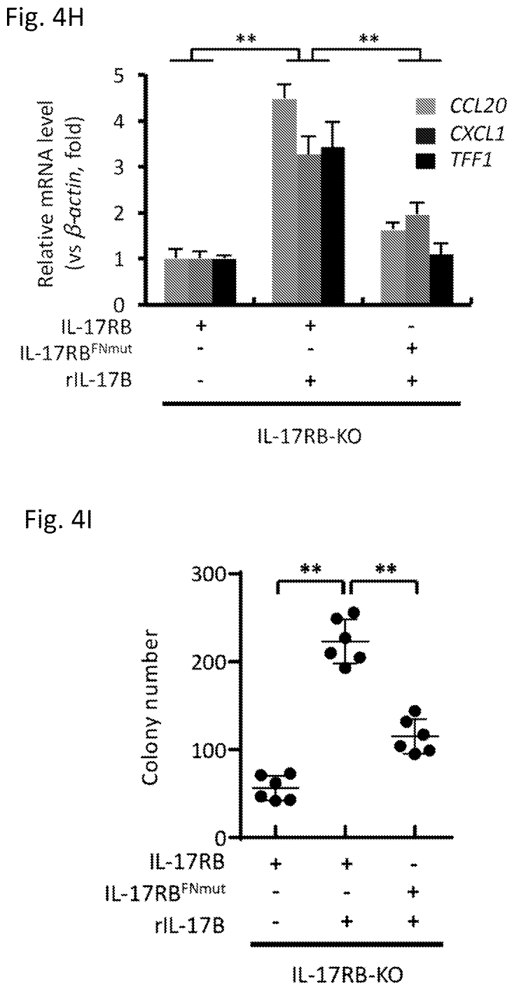

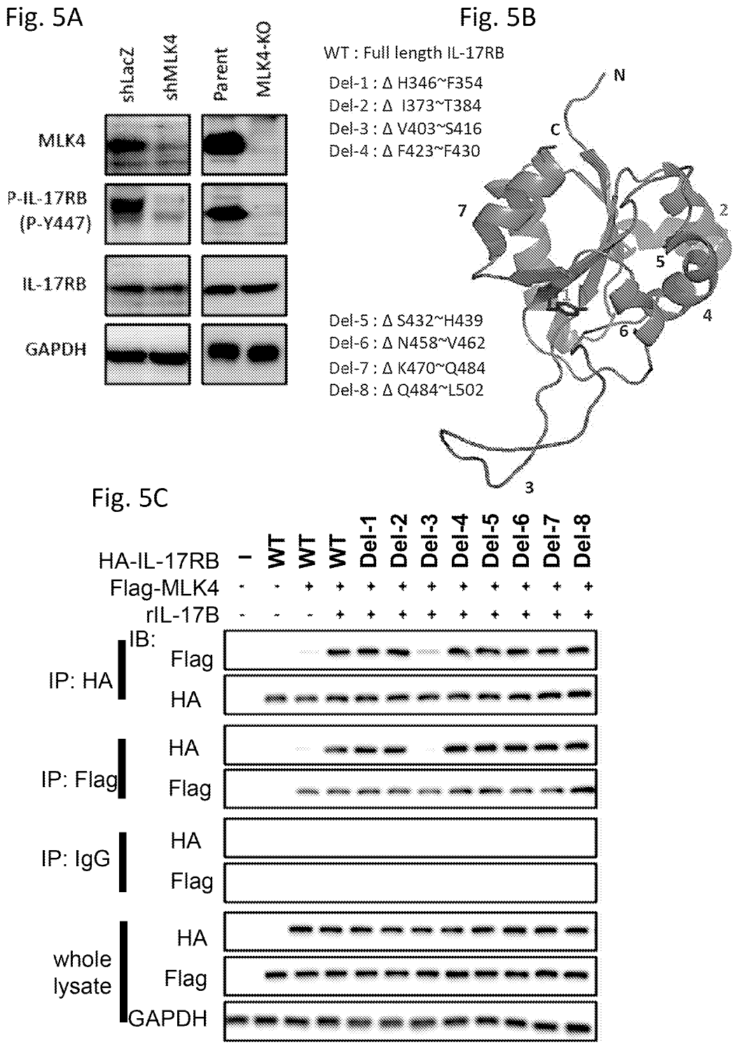

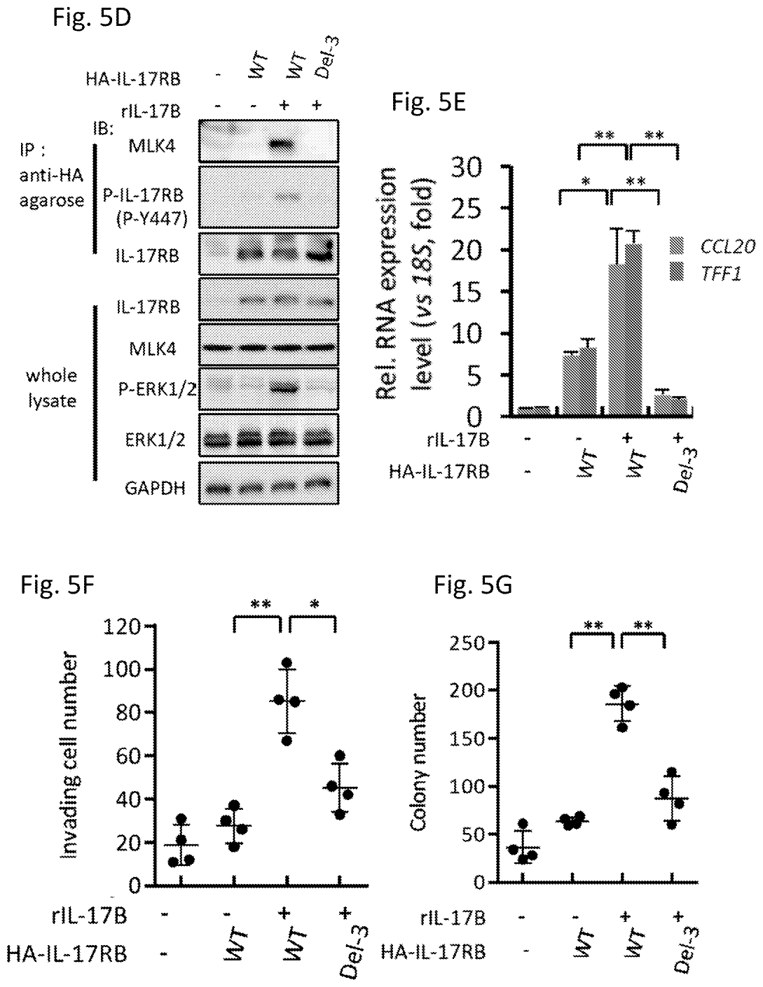

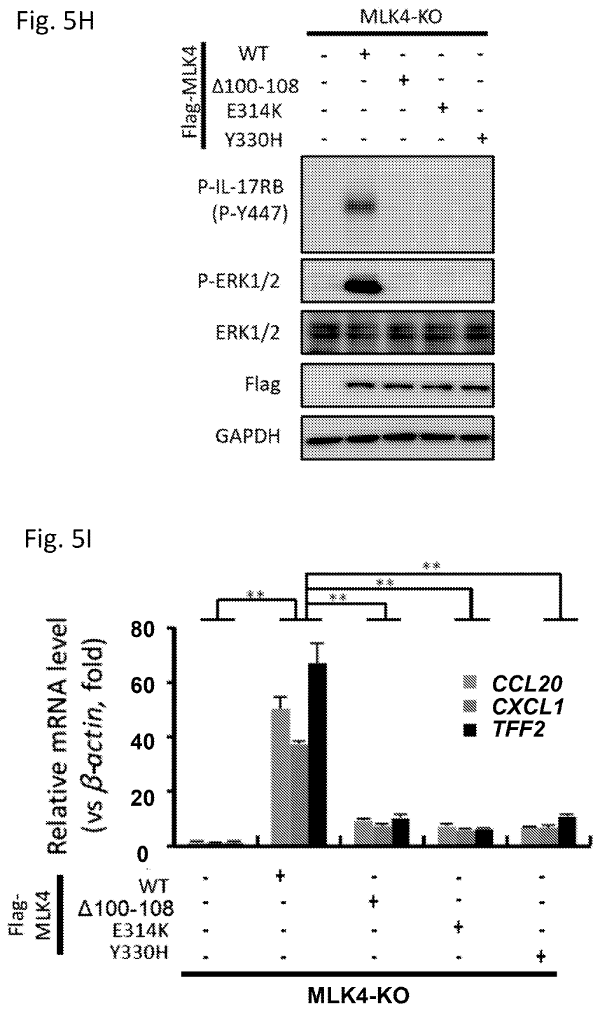

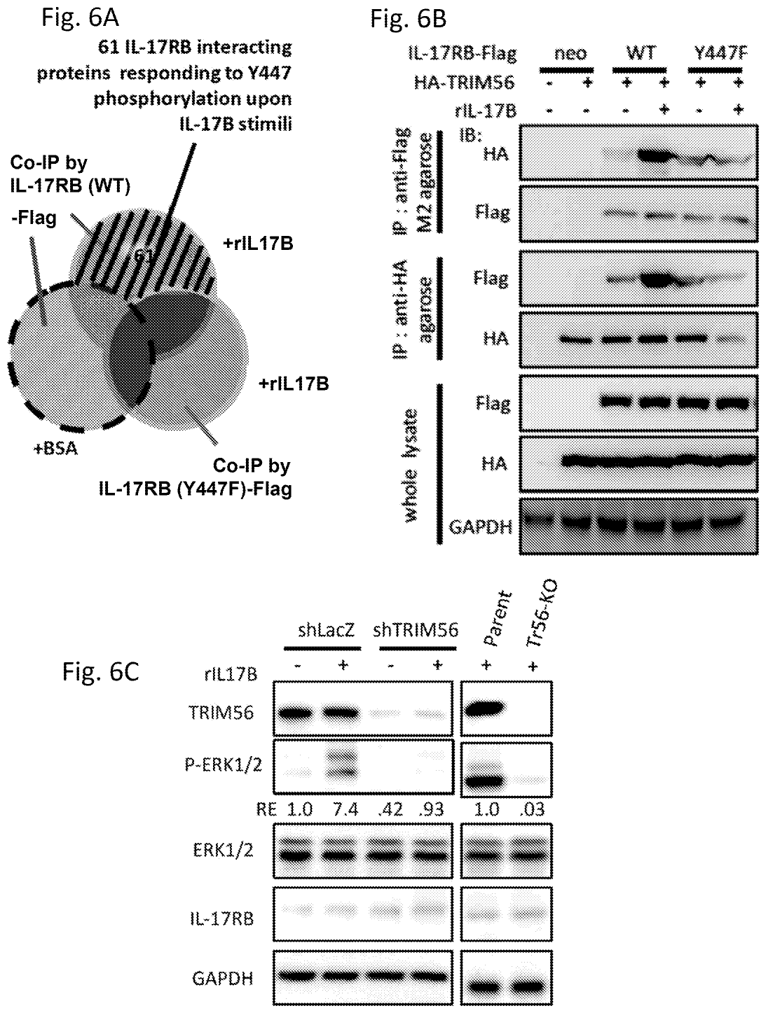

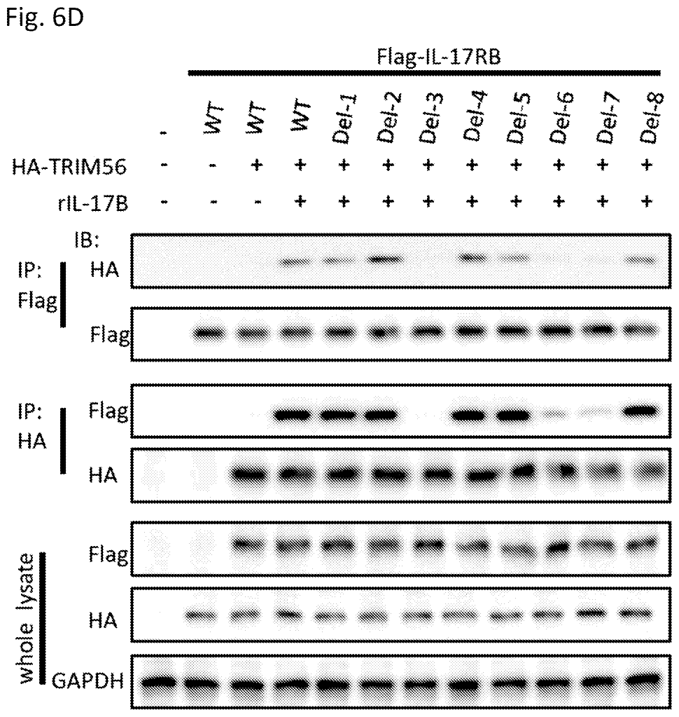

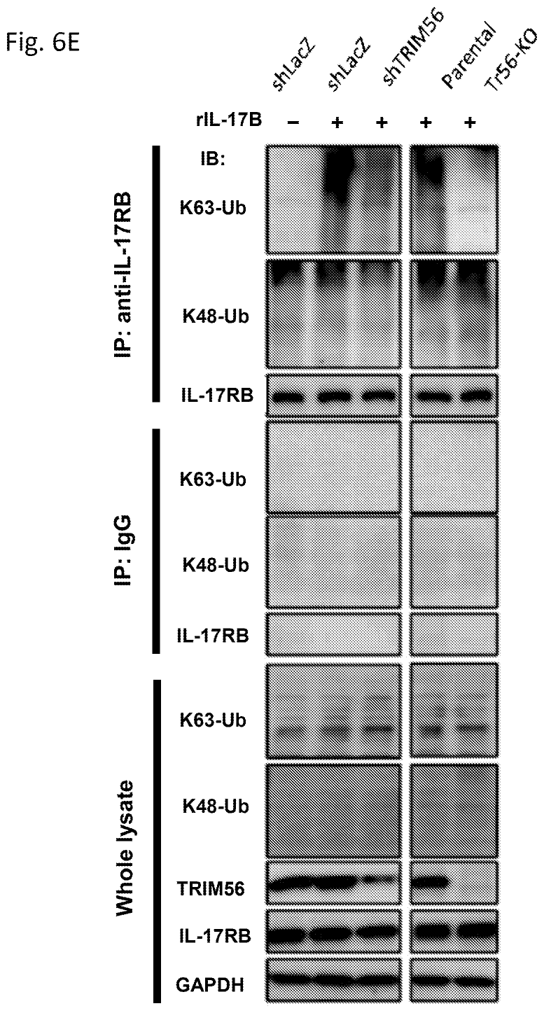

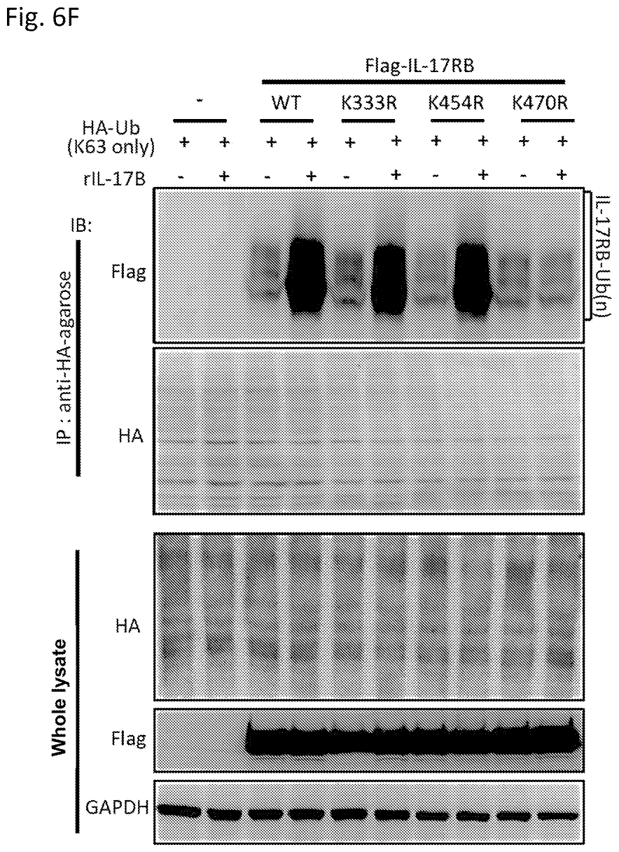

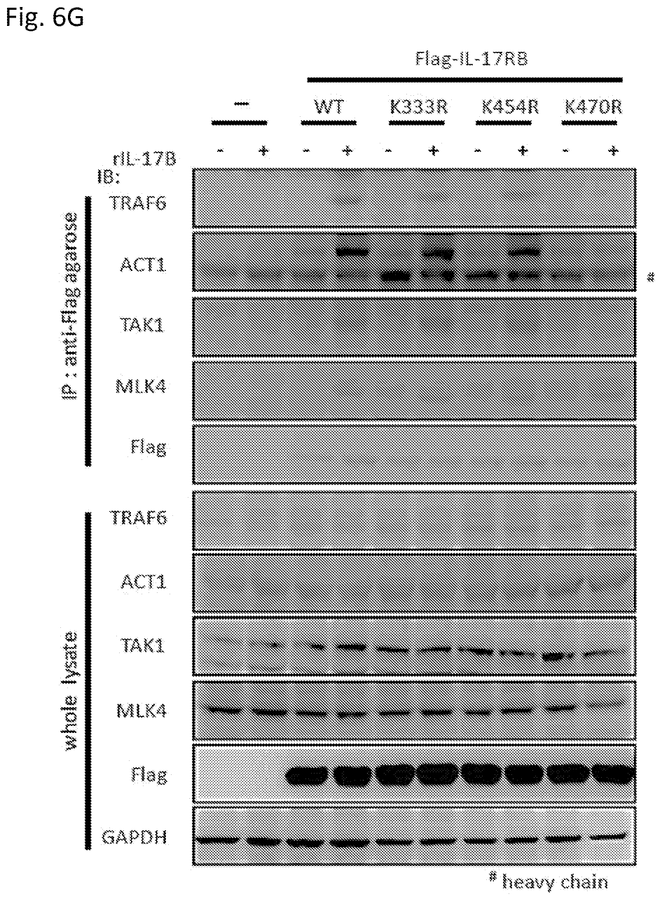

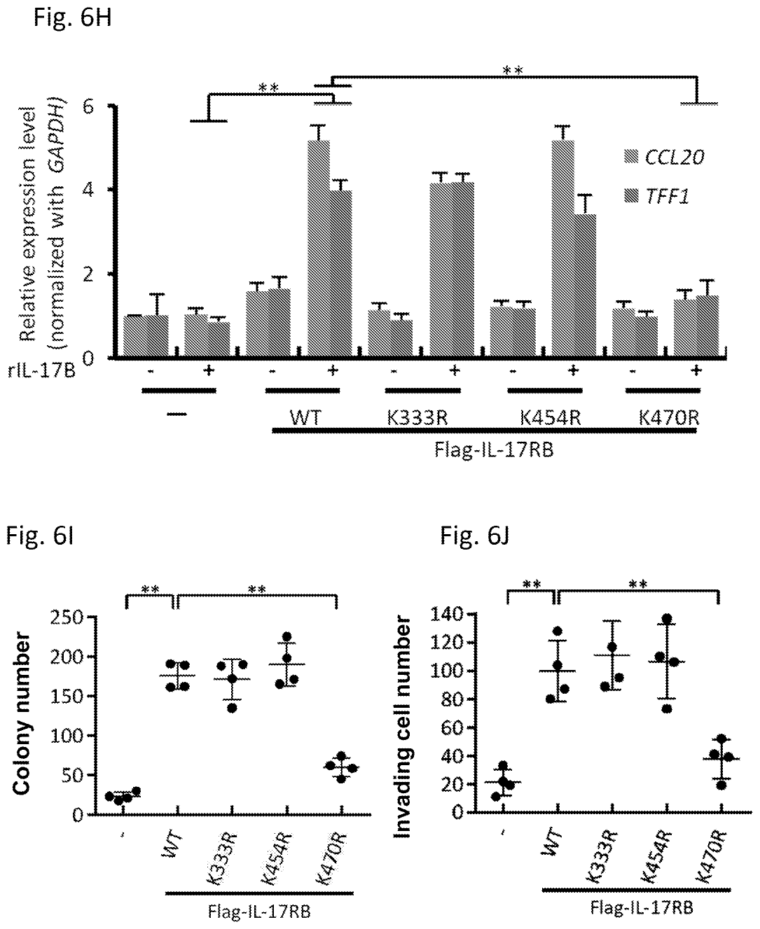

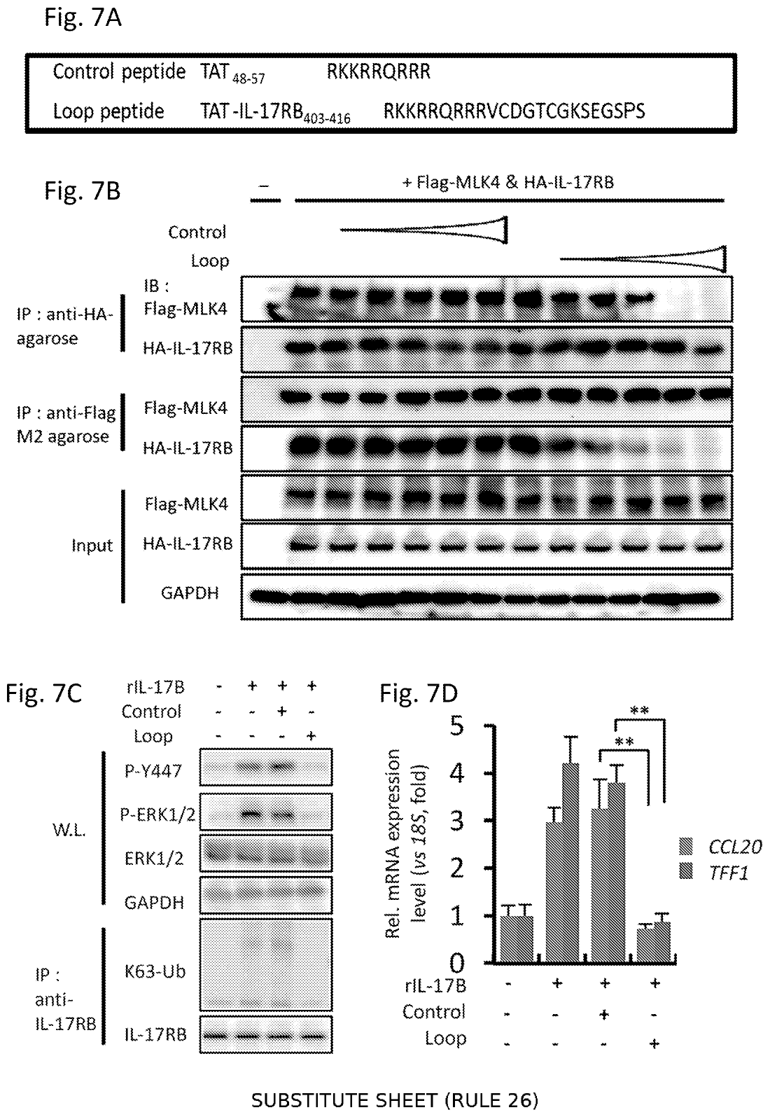

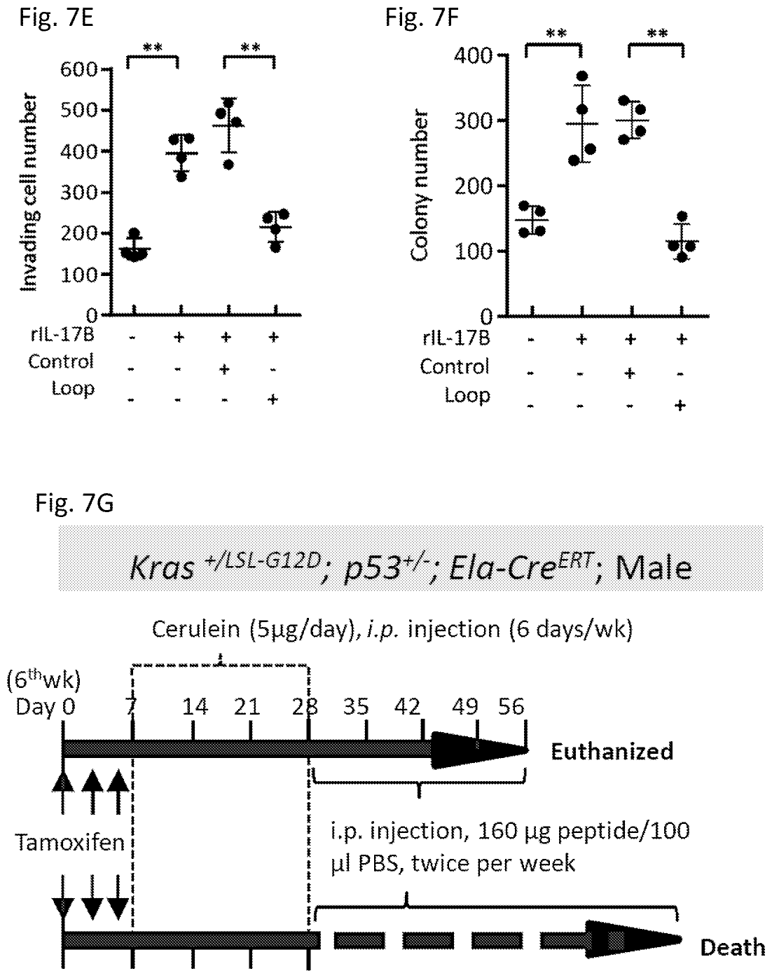

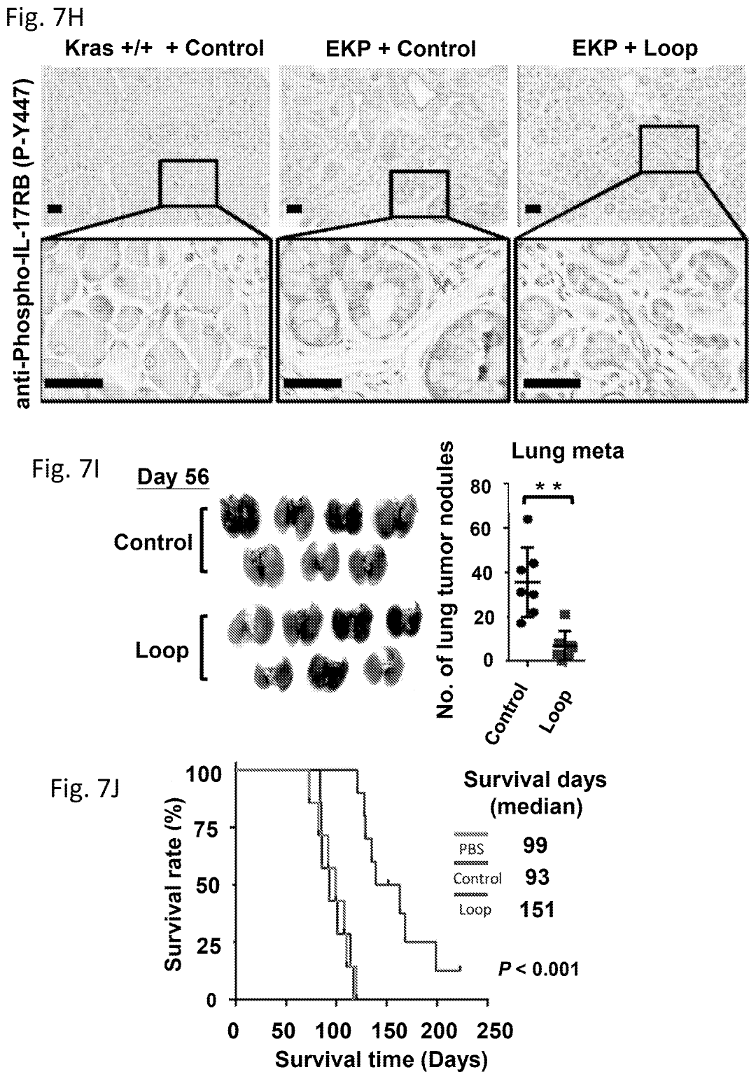

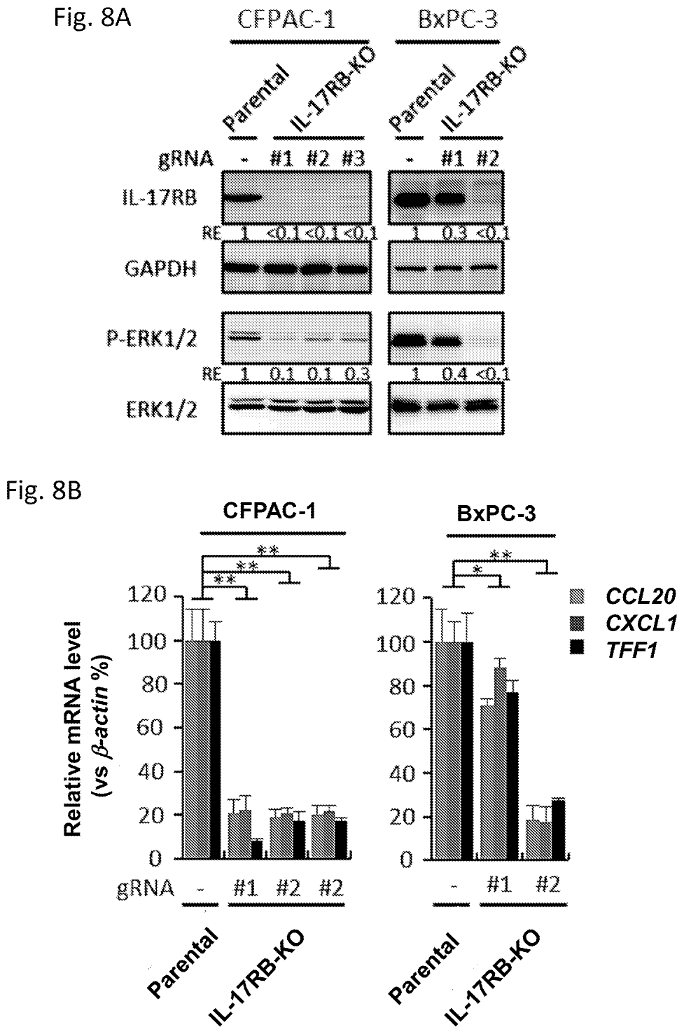

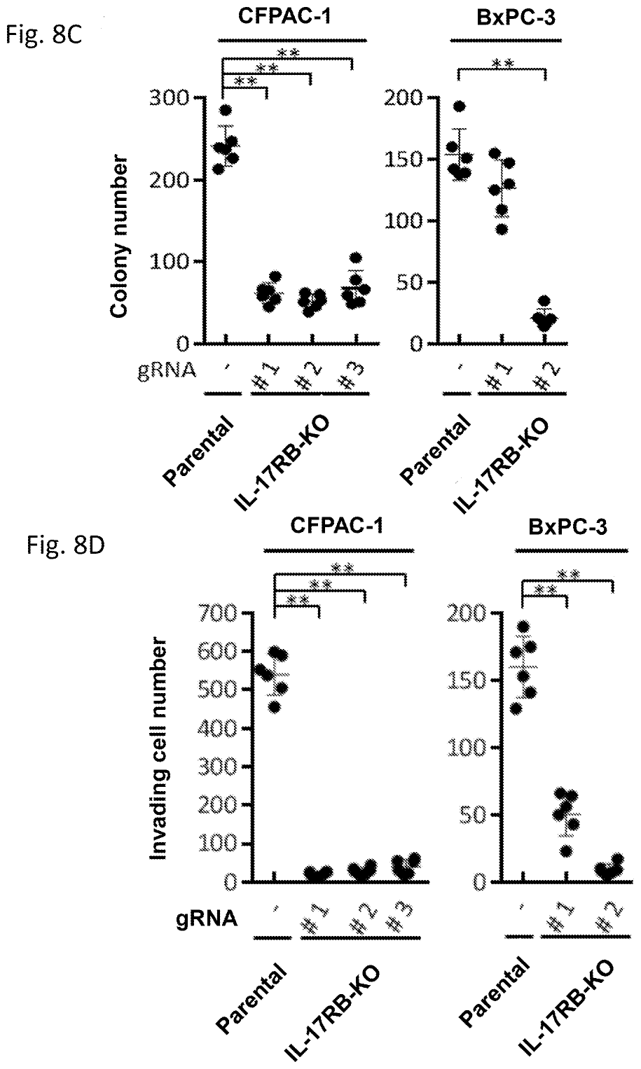

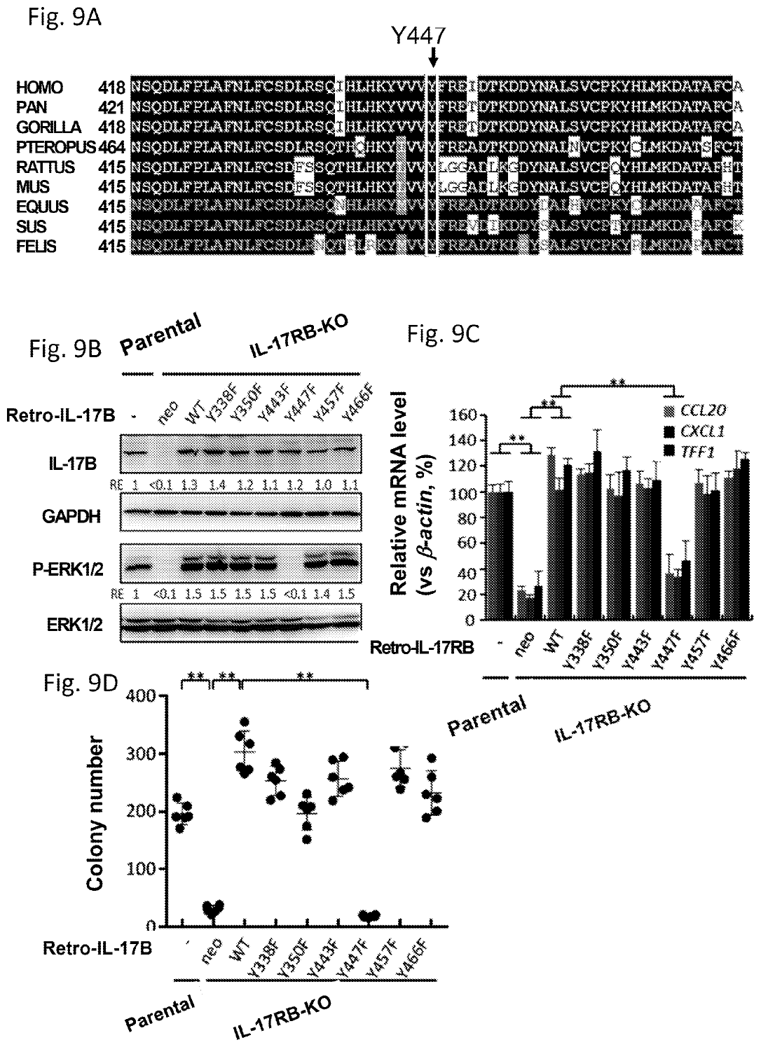

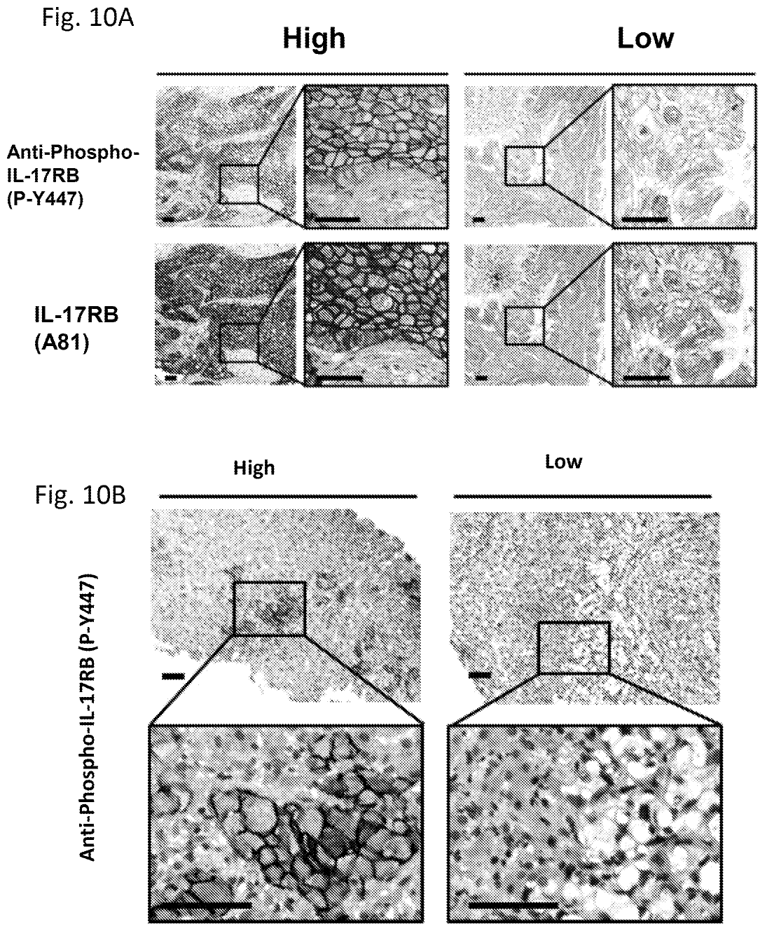

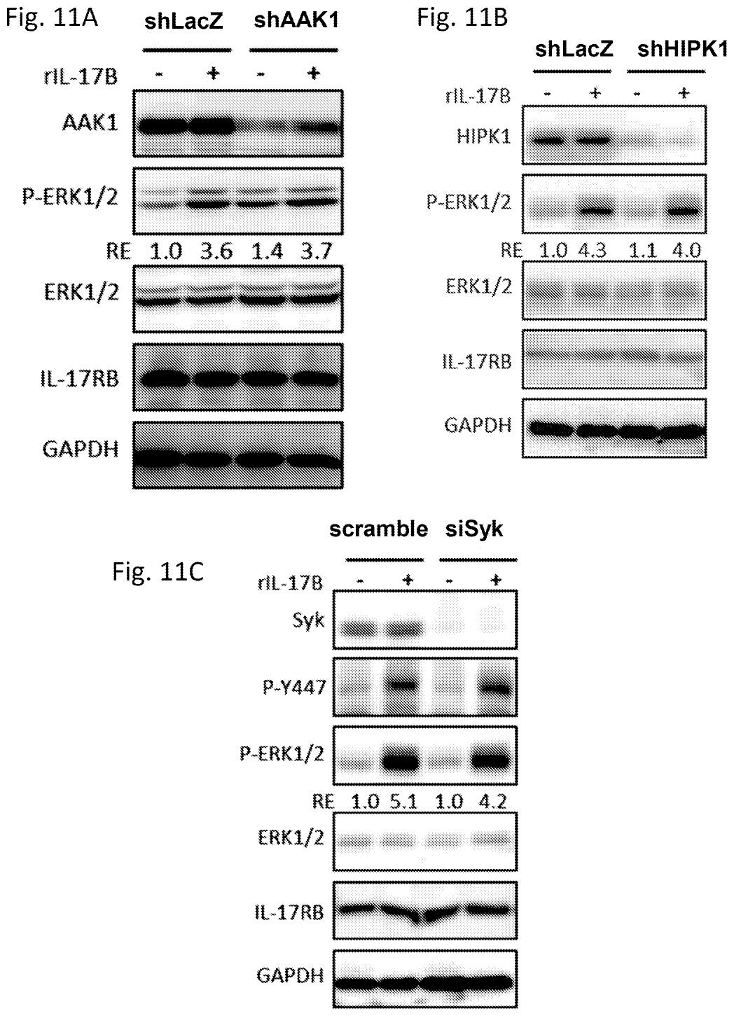

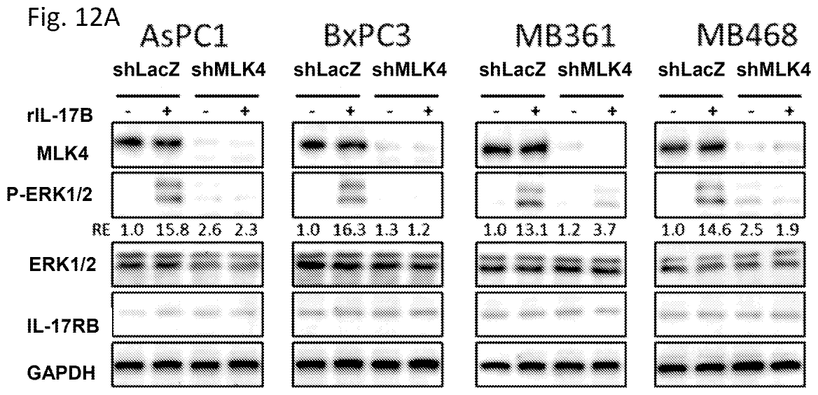

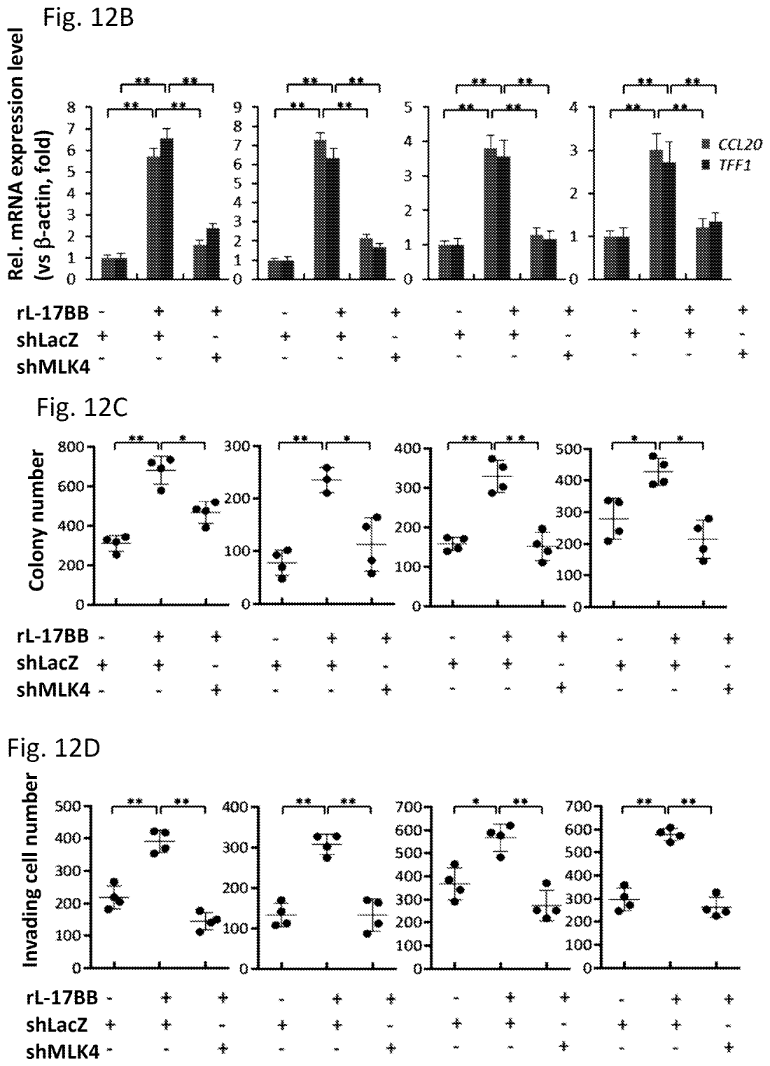

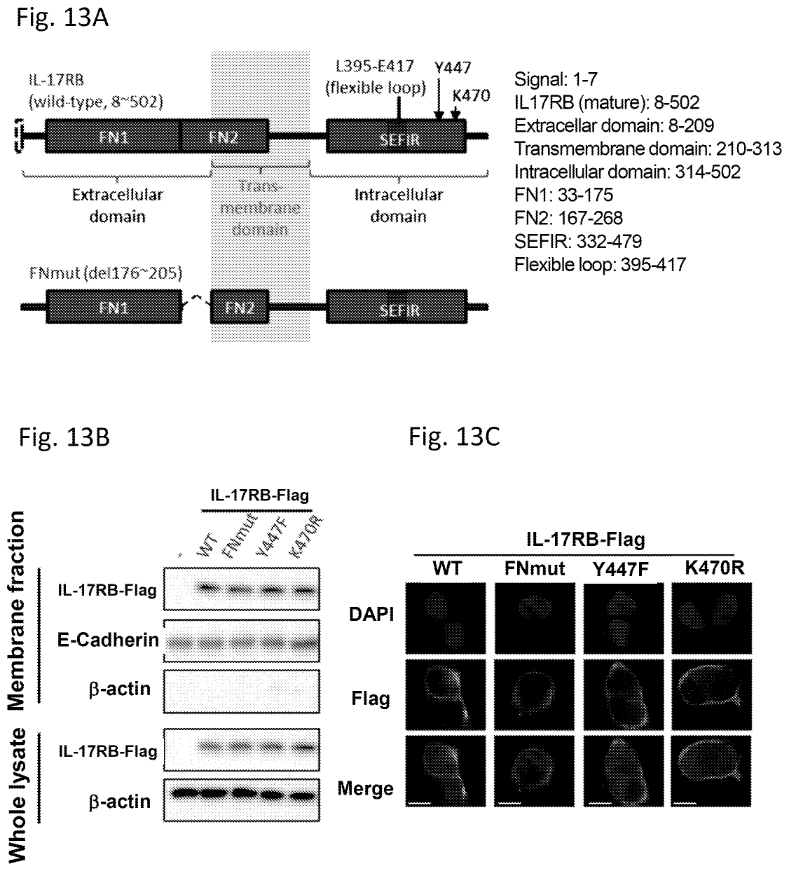

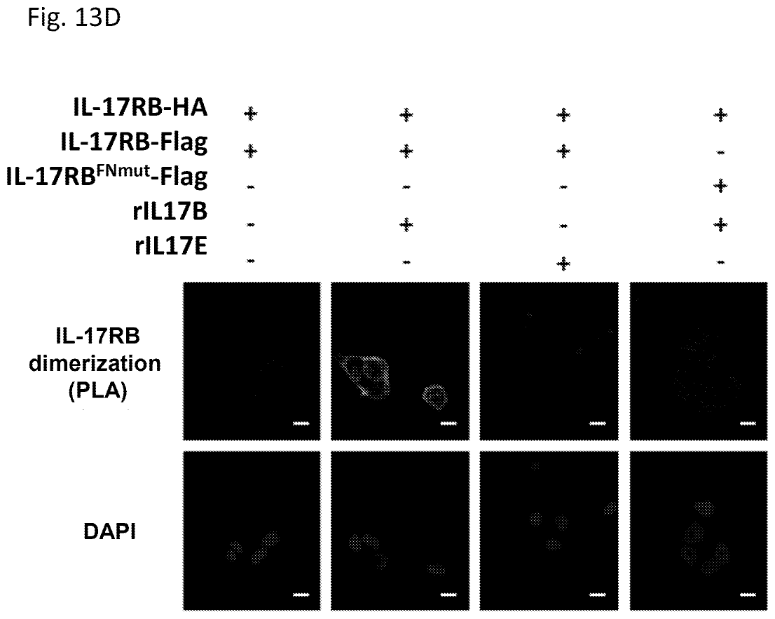

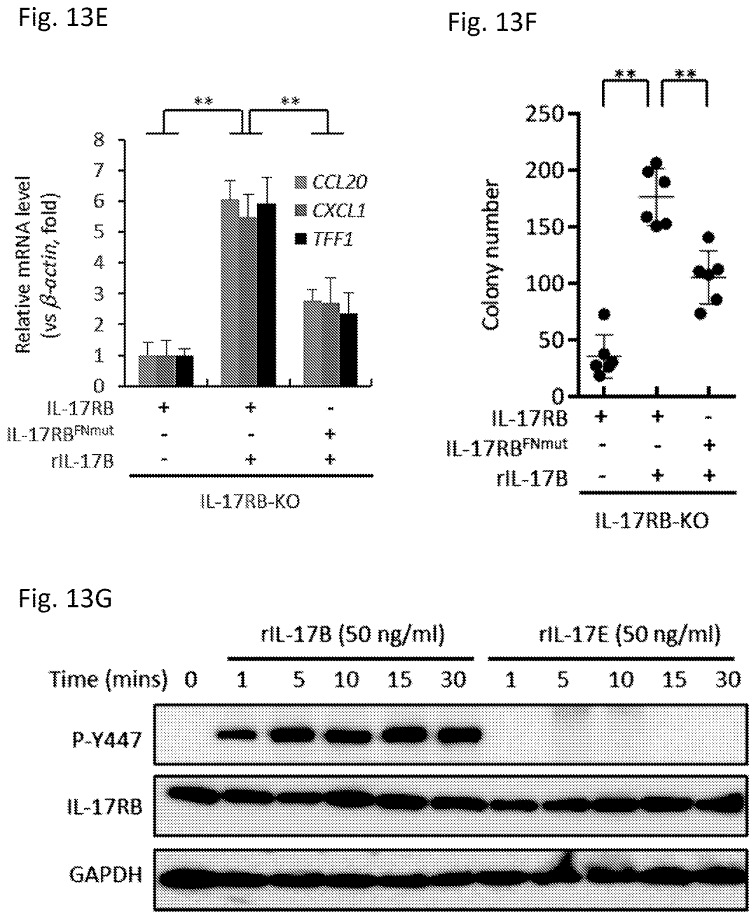

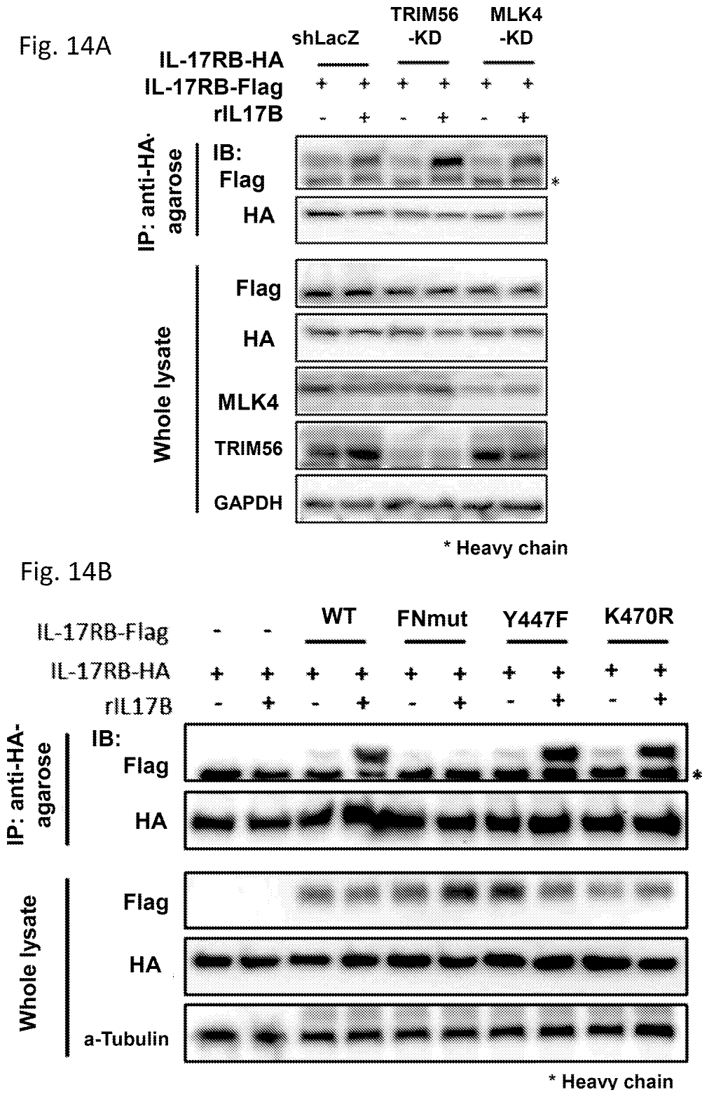

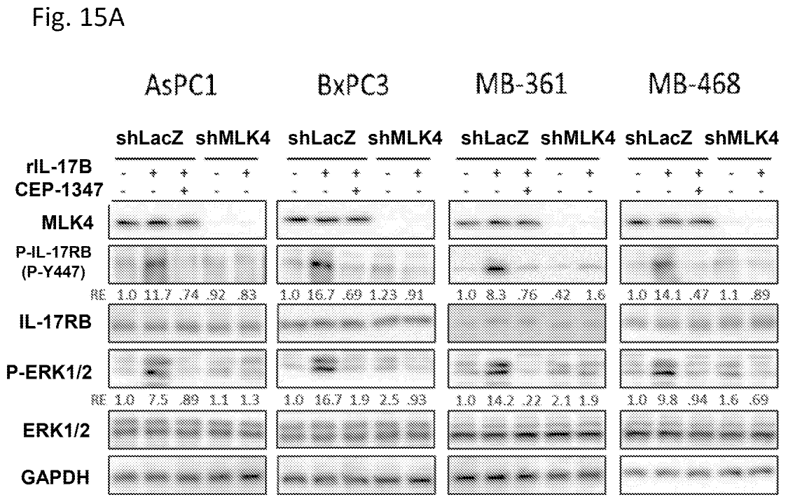

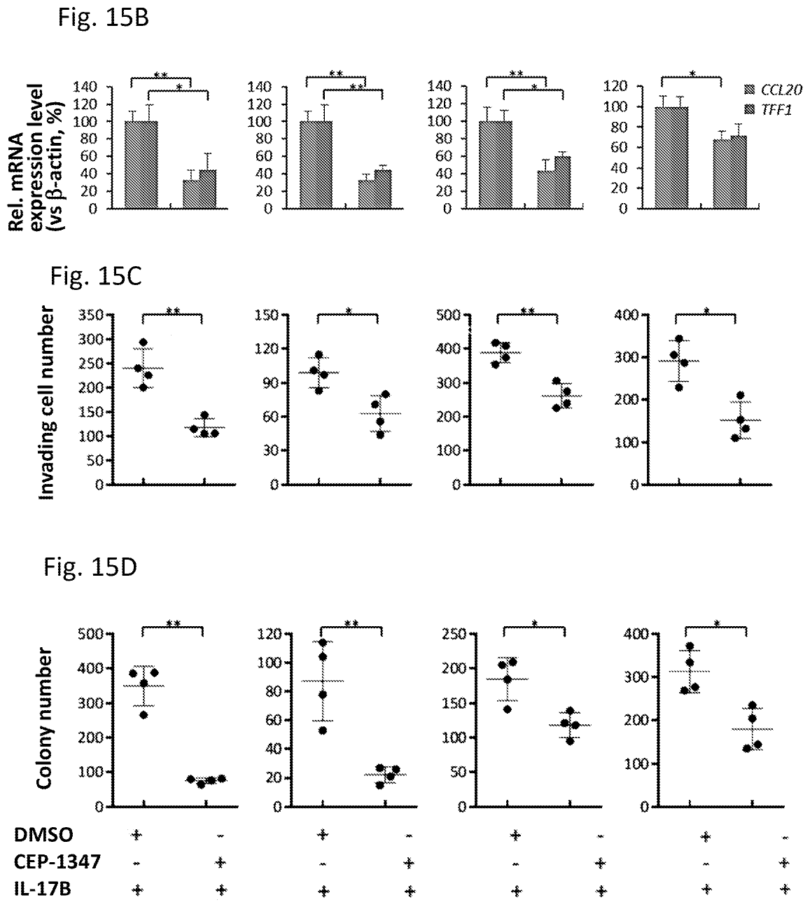

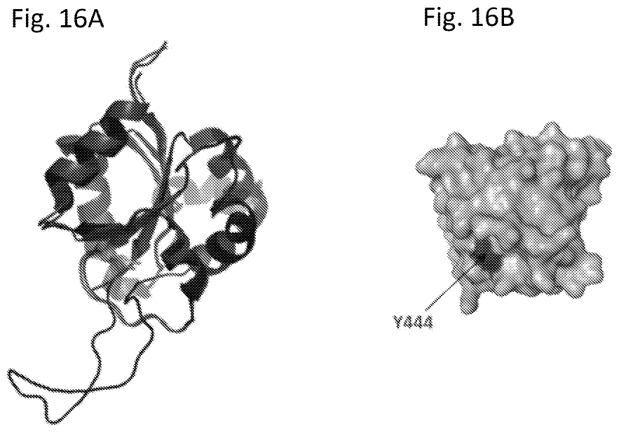

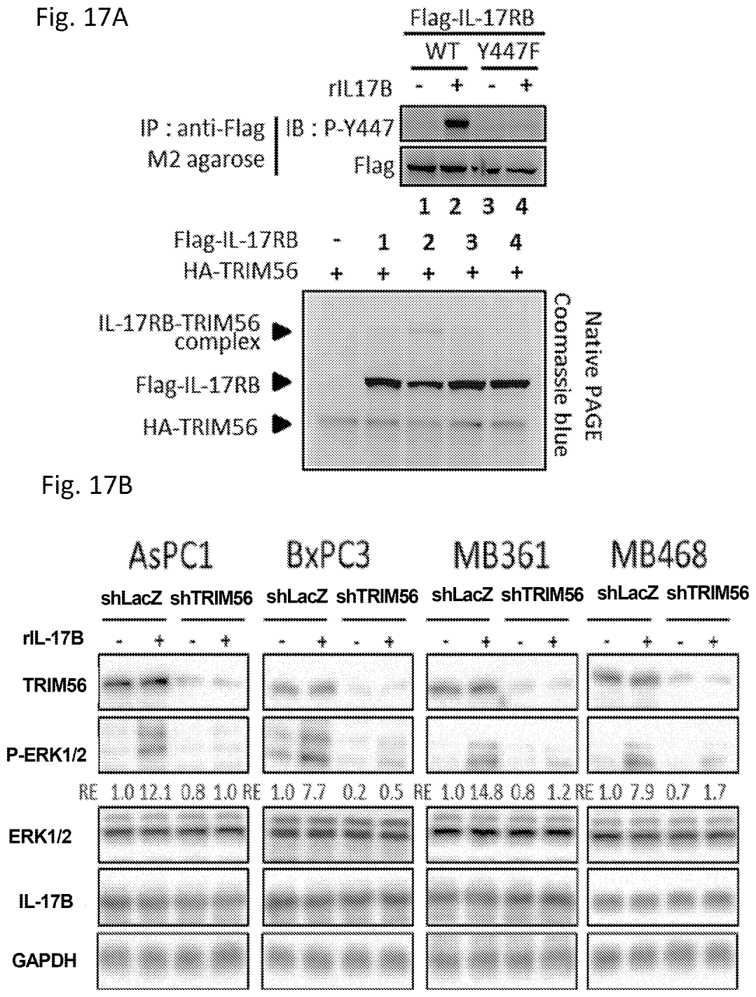

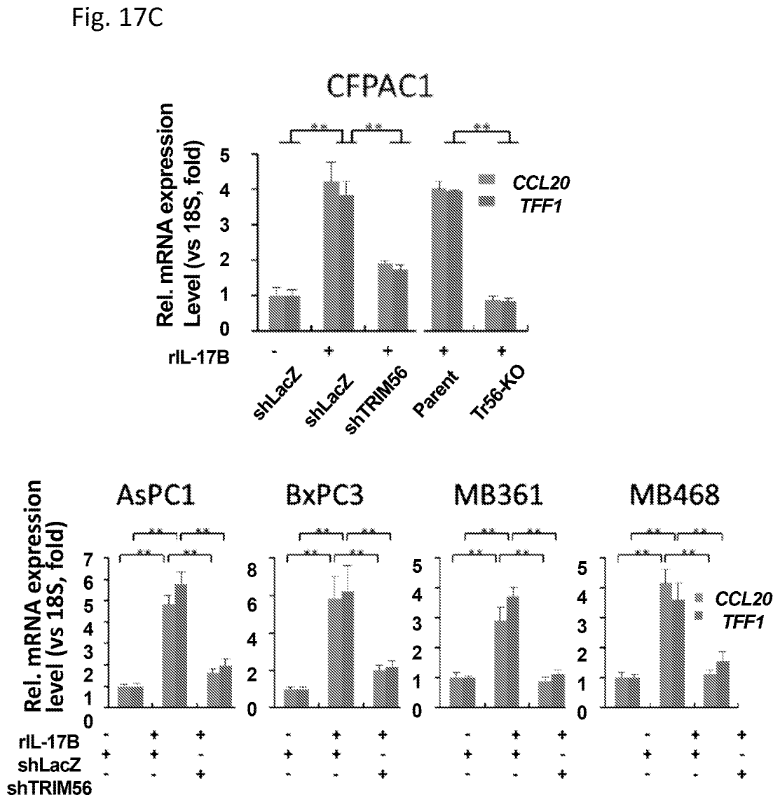

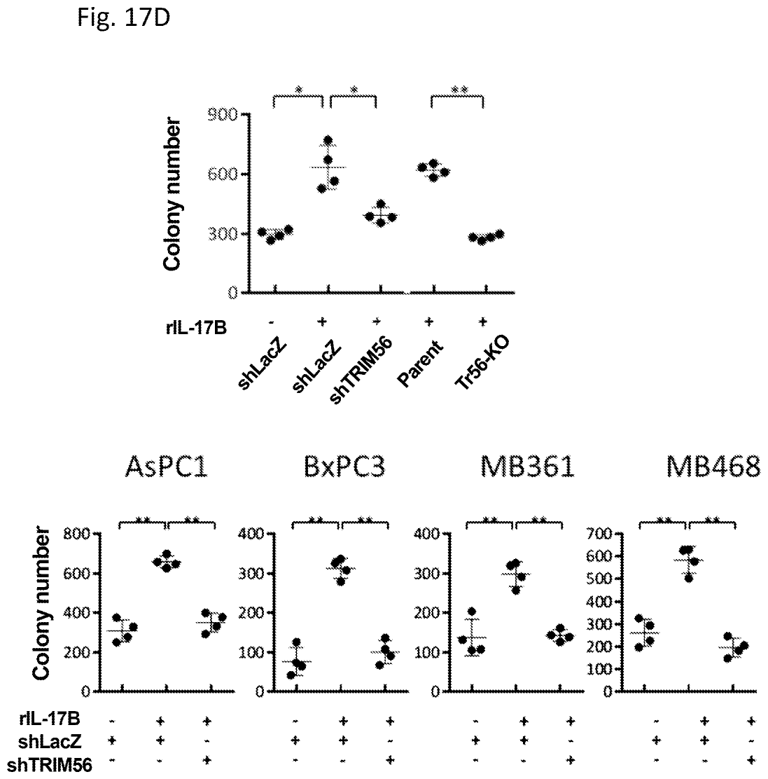

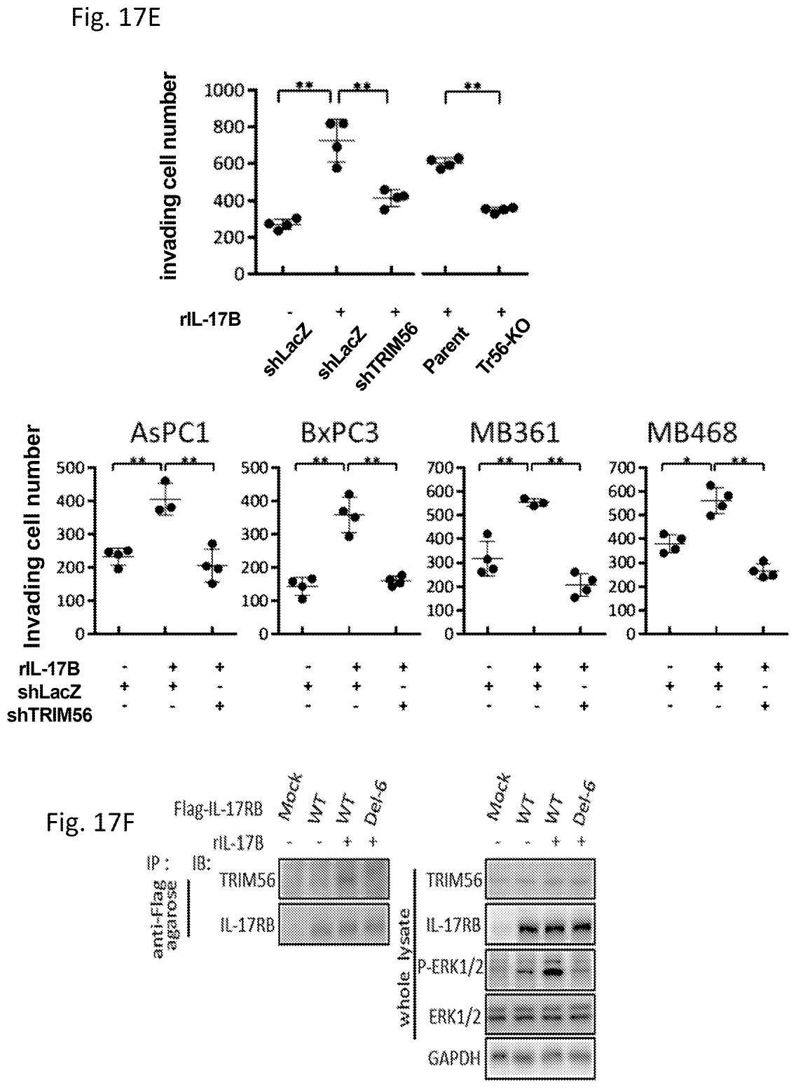

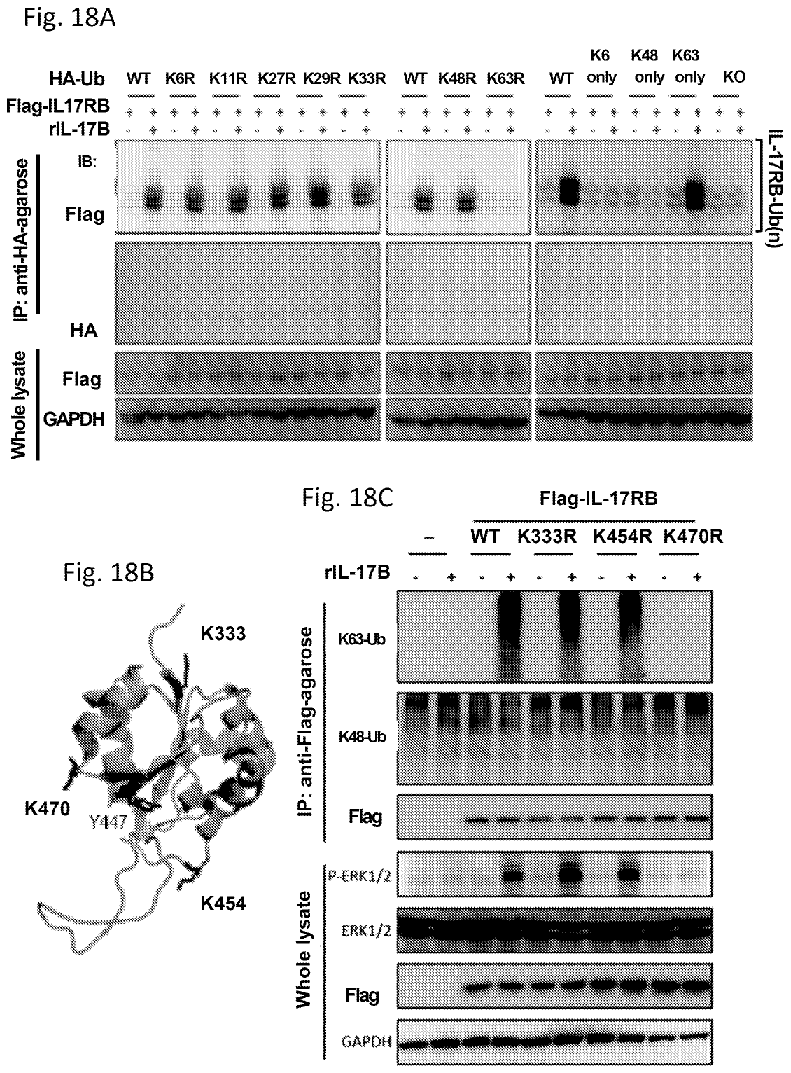

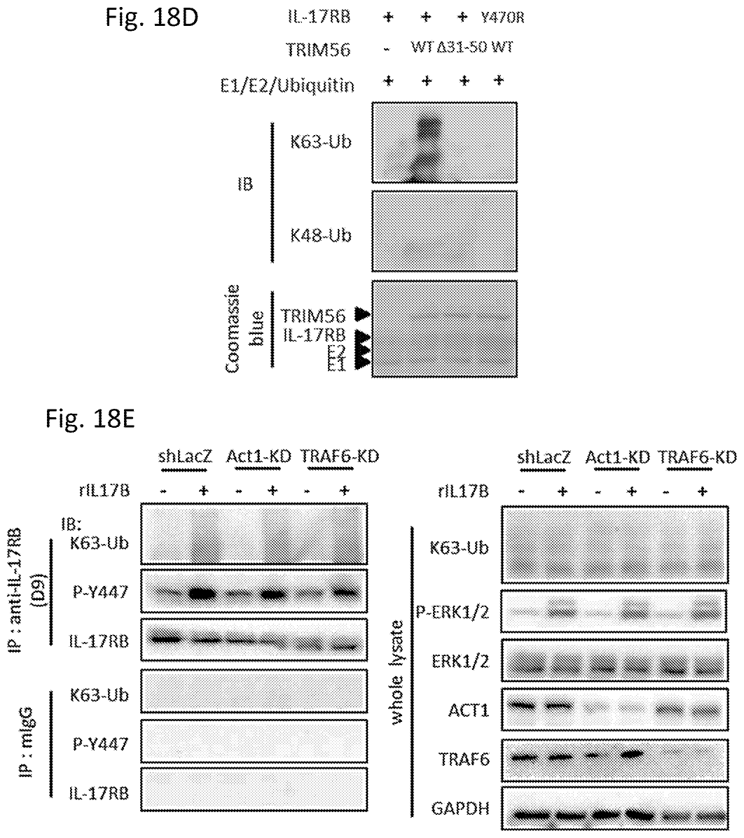

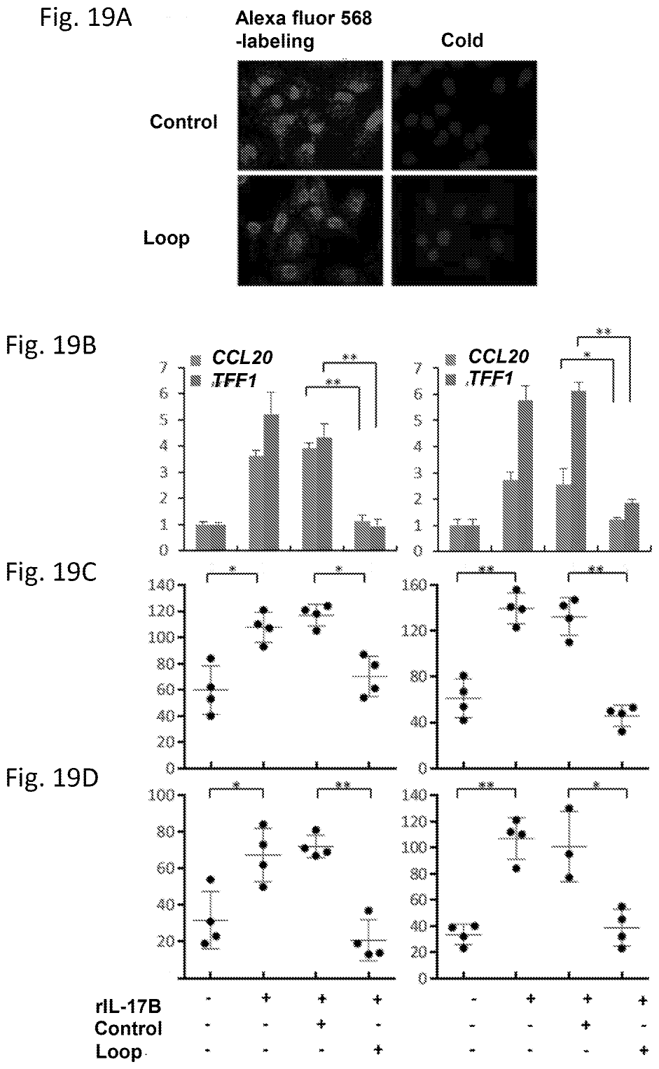

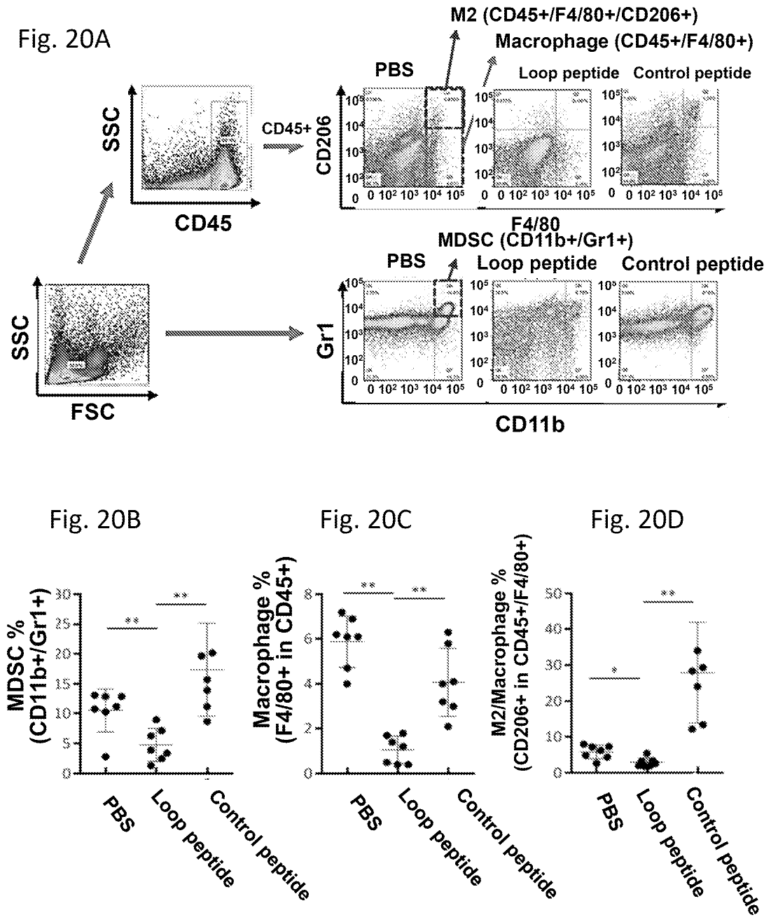

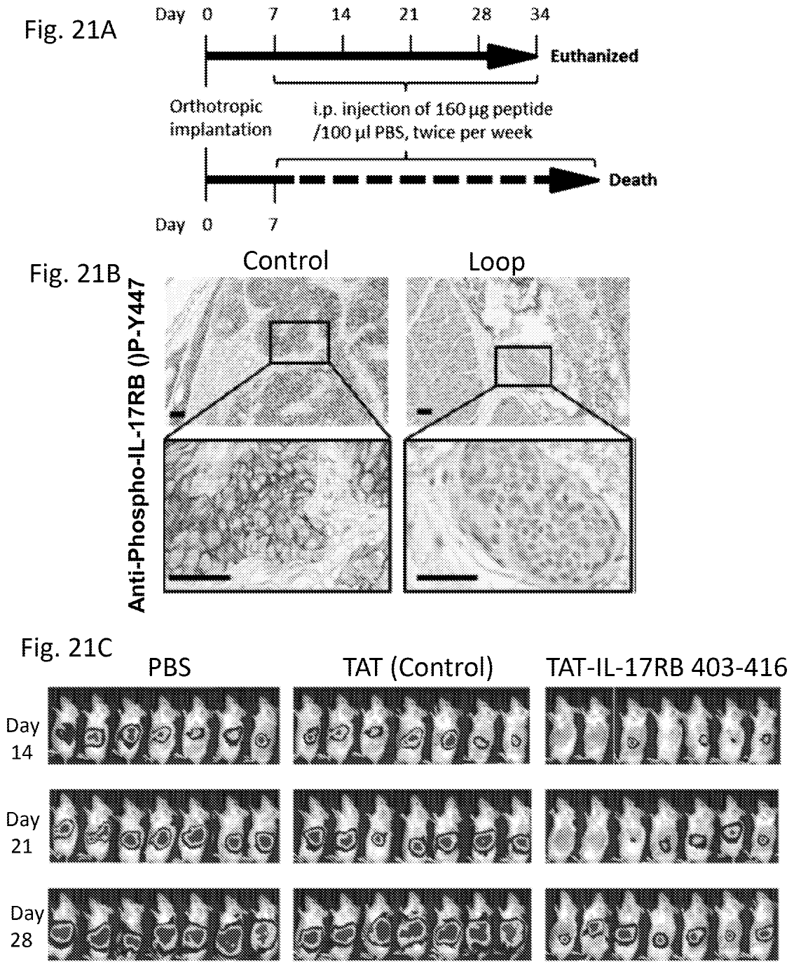

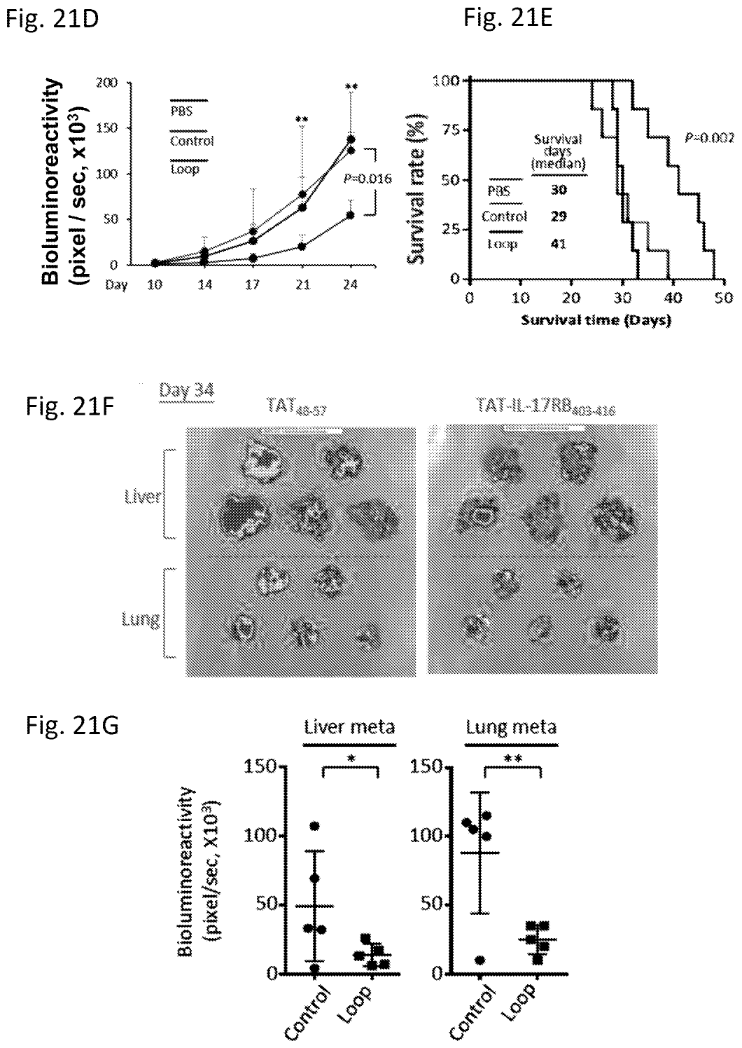

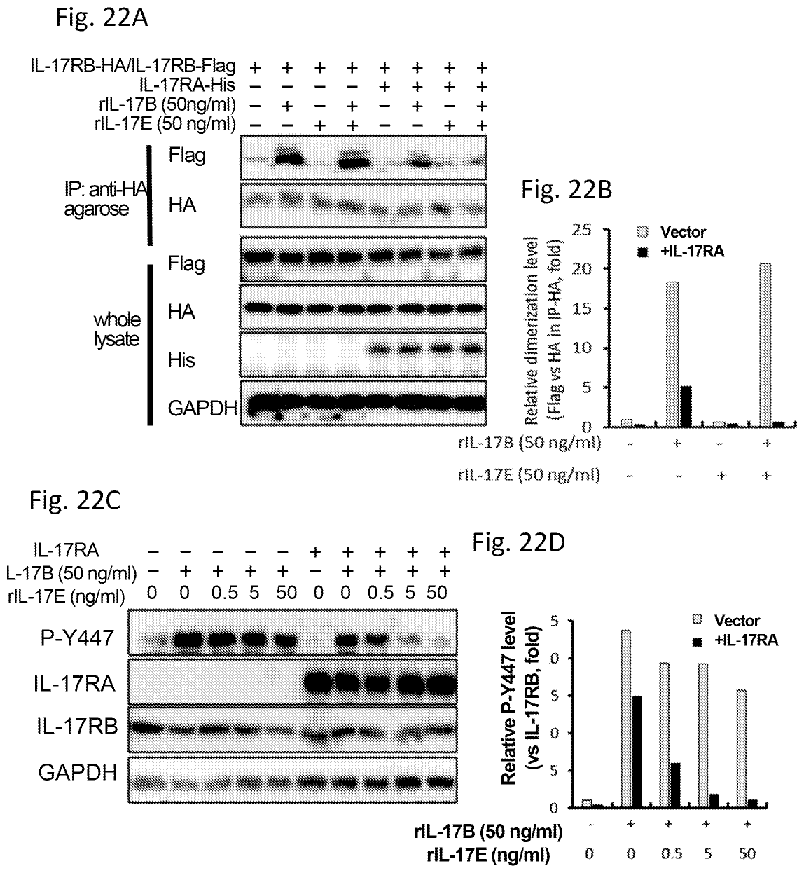

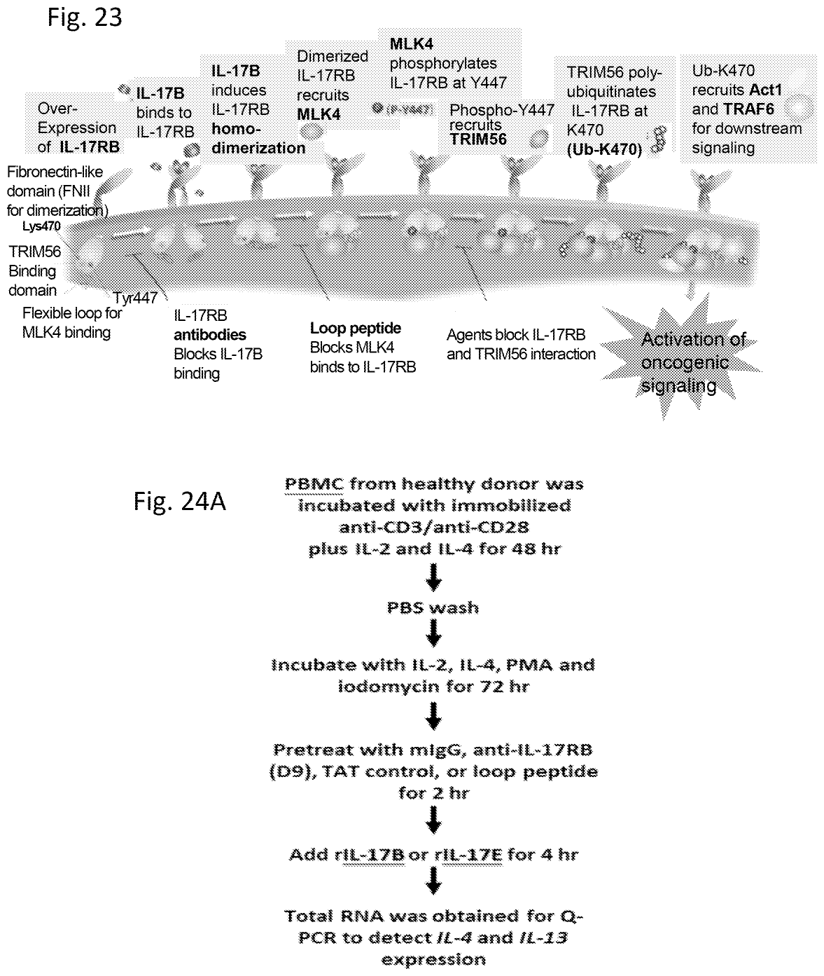

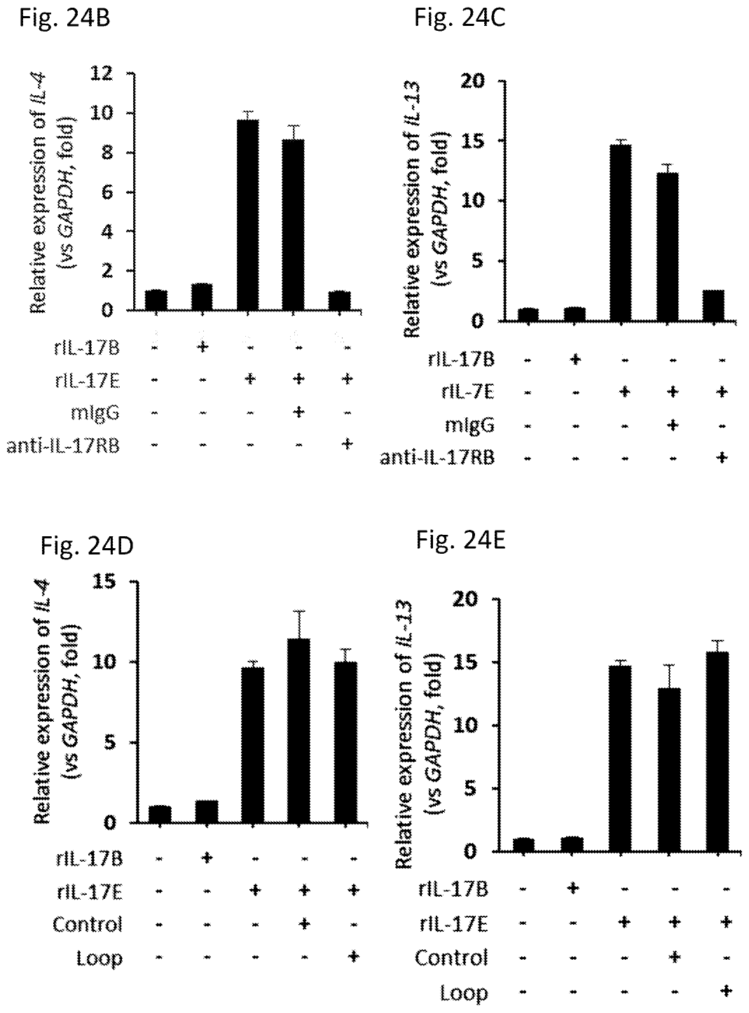

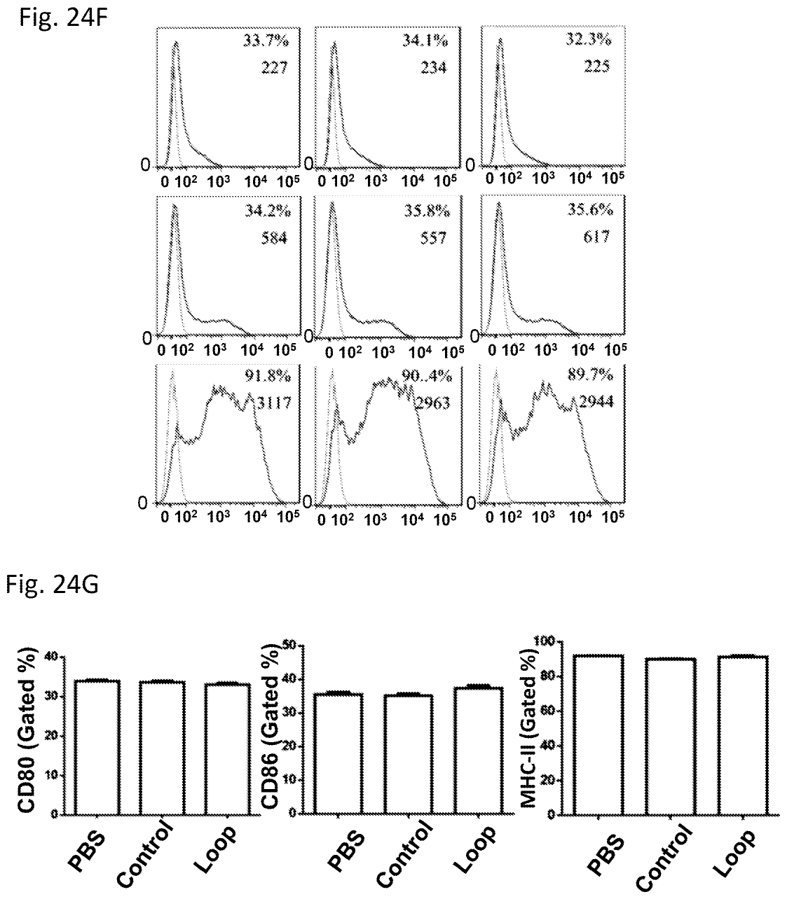

The diagram showed that 126 protein candidates were specifically identified upon rIL-17B treatment (right). (FIG. 3B) Immunoblotting analysis of IL-17RB binding to the three kinases. CFPAC1 cells were treated with the indicated concentrations of rIL-17B and the cell lysates were co-immunoprecipitated with anti-IL-17RB and blotted with the indicated antibodies or directly immunoblotted with the indicated antibodies as the input controls. (FIG. 3C) Immunoblotting analysis of MLK4 depletion and ERK1/2 phosphorylation. CFPAC1 cells was depleted by the corresponding lenti-shRNAs of shMLK4 or shLacZ as the control and the cell lysates were directly immunoblotted with the indicated antibodies. RE: relative amount. (FIG. 3D) Co-immunoprecipitation of MLK4 and IL-17RB. 293T cells co-transfected with IL-17RB-HA and Flag-MLK4 were treated with rIL-17B and the cell lysates were reciprocally co-immunoprecipitated with anti-Flag- and anti-HA-conjugated beads and followed by immunoblotting analysis with the indicated antibodies. (FIG. 3E) Image micrographs of Duolink in situ interaction assay. The interaction between IL-17RB with MLK4 was analyzed in BxPC3 cells using anti-IL-17RB and anti-MLK4 antibodies after rIL-17B treatment. Scale bars: 5 μm (upper). The green dots in the cells were scored (n=20) (bottom). (FIG. 3F and FIG. 3G) CFPAC1 cells with MLK4-knockdown was used for measuring cytokine gene mRNA expression by RT-qPCR (n=3) (FIG. 3F), or for SACF assay (n=4) (FIG. 3G).

FIGS. 4A to 4I include charts showing that homodimerization of IL-17RB recruits MLK4 for downstream oncogenic signaling. (FIG. 4A) Immunoblotting analysis of IL-17RB homodimerization induced by IL-17B. IL-17RB-KO BxPC3 cells co-transfected with IL-17RB-HA and IL-17RB-Flag were treated with rIL-17B for the indicated time. The cell lysates were immunoprecipitated with anti-Flag-conjugated beads and immunoblotted with the indicated antibodies or directly immunoblotted with the indicated antibodies as the input controls. (FIG. 4B) Immunoblotting analysis of IL-17RB^{FNmut} mutant homodimerization. 293T cells expressing IL-17RB-HA was co-transfected with IL-17RB-Flag or IL-17RB^{FNmut}-Flag and treated with rIL-17B and the cell lysates were immunoprecipitated with anti-HA-conjugated beads and immunoblotted with the indicated antibodies or directly immunoblotted with the indicated antibodies as the input controls. (FIG. 4C) Duolink in situ interaction assay. IL-17RB-KO BxPC3 expressing IL-17RB-HA expressing IL-17RB-Flag or IL-17RB^{FNmut}-Flag were treated with IL-17B or IL-17E and subjected for Duolink in situ interaction assays using anti-HA and anti-Flag as probes. The plot shows the numbers of the positive green dots. (FIG. 4D) Duolink in situ interaction assay of the endogenous IL-17RB in response to IL-17B. CFPAC1 cells treated with 50 ng/ml rIL-17B for 30 minutes were stained with FITC-prelabled anti-IL-17RB (D9), following Duolink in situ interaction assay with two mouse secondary antibodies carrying PLA probes (anti-mouse PLUS and anti-mouse MINUS). Dimerization of IL-17RB upon rIL-17B treatment was revealed by the red dots at the membrane (left). Scale bars: 5 μm. Quantification of the red dots (n=25) was shown in right panel. (FIG. 4E) Immunoblot analysis of IL-17RB^{FNmut} binding to MLK4 upon IL-17B stimuli. The cells, same as in (C), were treated with 50 ng/ml rIL-17B or rIL-17E for 30 mins and the cell lysates were immunoprecipitated with anti-Flag-conjugated beads and immunoblotted with the indicated antibodies or directly immunoblotted with the indicated antibodies as the input controls. (FIG. 4F) Immunoblot analysis of IL-17RB^{FNmut} in IL-17B-induced IL-17RB Y447 and ERK1/2 phosphorylation. The same cells as (FIG. 4C) were lysed and the whole cell lysates were directly immunoblotted with the indicated antibodies. (FIG. 4G) Immunoblot analysis of distinct dimerization pattern of IL-17RB upon IL-17B stimuli. IL-17RB-KO BxPC3 cells were co-transfected with IL-17RB-HA, IL-17RB-Flag and IL-17RA-His and treated with rIL-17B or rIL-17E and the cell lysates were immunoprecipitated with anti-HA-conjugated beads following immunoblotted with the indicated antibodies or directly immunoblotted with the indicated antibodies as the input control. (FIG. 4H). Relative expression of CCL20, CXCL1 and TFF1 mRNA in IL-17RB-KO CFPAC1 cells. IL-17RB-KO CFPAC1 cells expressing IL-17RB-Flag, or IL-17RB^{FNmut}-Flag were treated with rIL-17B for measuring the corresponding mRNA by RT-qPCR (n=3) or for SACF assay (n=6) (FIG. 4I).

FIGS. 5A to 5I include charts showing that the flexible loop (V403˜S416) of IL-17RB is required for MLK4 binding and Y447-phosphorylation of IL-17RB. (FIG. 5A) Immunoblotting analysis of MLK4 activity for phosphorylation of Y447 of IL-17RB. BxPC3 cells either treated with shMLK4, or shLacZ or depleted MLK4 by knockout were induced with rIL-17B, and the cell lysates were immunoblotted with the indicated antibodies. (FIG. 5B) Diagram of the human IL-17RB intracellular domain with eight potential subdomains and the list of mutants with the corresponding deleted amino acid sequences. (FIG. 5C) Immunoblotting analysis of the subdomain of IL-17RB essential for MLK4 binding. 293T cells were co-transfected with Flag-MLK4 and HA-tagged wild-type (WT) or each mutant listed above and then treated with rIL-17B. The cell lysates were reciprocally co-immunoprecipitated with anti-HA and anti-Flag and blotted with anti-Flag or anti-HA or directly blotted with the indicated antibodies as the input controls. (FIG. 5D) Immunoblot analysis of Del-3 mutant binding to MIL4 and phosphorylating Y447 and ERK1/2. The HA-tagged WT or Del-3 mutant of IL-17RB were transduced into the low IL-17RB-expressing SU.86.86 cells, respectively. After rIL-17B treatment for 30 min, the cell lysates were subjected to co-IP with anti-HA-conjugated beads followed by immunoblotting with anti-P-Y447 and anti-MLK4 or directly immunoblotted with the indicated antibodies as the input controls. (FIG. 5E to FIG. 5G) Biological activity assay of Del-3 mutant. CCL20 and TFF1 mRNA of IL-17RB-KO BxPC3 cells expressing HA-tagged WT or Del-3 mutant of IL-17RB were measured by RT-qPCR (n=3) (FIG. 5E), for invasion assay (n=4) (FIG. 5F), and for SACF assay (n=4) (FIG. 5G) after treated with 50 ng/ml rIL-17B. (FIG. 5H and FIG. 5I), Immunoblotting analysis of MLK4 kinase mutants in IL-17B-induced oncogenic activity. Flag-tagged WT and kinase mutants (Δ130-138, E314K, Y330H) of MLK4 were separately expressed in MLK4-KO BxPC3 cells following rIL-17B treatment. The corresponding cell lysates were directly immunoblotted with the indicated antibodies (FIG. 5H). (FIG. 5I) CCL20, CXCL1 and TFF1 mRNA expressions of the same cells as (FIG. 5H) were measured by RT-qPCR (n=3).

FIGS. 6A to 6J include charts showing that P-Y447 IL-17RB recruits TRIM56 for K63-linked ubiquitination at K470 of IL-17RB for downstream oncogenic signaling. (FIG. 6A) Diagram illustrated the process of the identification of 61 protein candidates specifically interacting with WT, but not Y447F mutant of IL-17RB after rIL-17B treatment. IL-17RB-KO BxPC3 cells expressing Flag-tagged WT or Y447F mutant of IL-17RB were treated with rIL-17B. These cells were used for the co-immunoprecipitation with anti-Flag and then followed by mass spectrometry analysis. (FIG. 6B) Immunoblotting analysis TRIM56 binding to IL-17RB. 293T cells expressing both Flag-tagged WT or Y447F of IL-17RB and HA-tagged TRIM56 were treated with rIL-17B and the cell lysates were co-immunoprecipitated and immunoblotted with the indicated antibodies or directly immunoblotted with the indicated antibodies as the input control. neo: empty vector. (FIG. 6C) Immunoblotting analysis of TRIM56 in ERK phosphorylation in responding to IL-17B. CFPAC1 cells treated with shTRIM56 or with TRIM56 knockout (Tr56-KO) were subject to rIL-17B stimuli. The corresponding cell lysates were immunoblotted with the indicated antibodies. RE indicates the relative expression of the phosphorylated ERK1/2. (FIG. 6D) Immunoblotting analysis of IL-17RB subdomain required for TRIM56 binding. 293T cells were co-transfected with HA-TRIM56 and Flag-tagged WT or each of the eight deletion mutants as in (FIG. 5B). The cells were lysed after treated with rIL-17B for reciprocal co-IP with anti-HA and anti-Flag and immunoblotted with the indicated antibodies or directly immunoblotted with the indicated antibodies as the input controls. (FIG. 6E) Blotting analysis of TRIM56 ligase activity for ubiquitination of IL-17RB. CFPAC1 cells either with TRIM56-knockdown (left), and TRIM56-knockout (right) were treated with rIL-17B and the cell lysates were used for co-immunoprecipitation with the indicated antibodies following immunoblotting with anti-K63-Ub, anti-K48-Ub and anti-IL-17RB antibodies or directly immunoblotted with the indicated antibodies as the input controls. (FIG. 6F) Immunoblotting analysis of lysine site of IL-17RB ubiquitinated by TRIM56. IL-17RB-KO CFPAC1 cells were co-transduced with Flag-tagged WT or mutant (K333R, K454R, K470R) of IL-17RB and HA-Ub (K63 only) and treated rIL-17B. The cell lysates were immunoprecipitated by HA-agarose following immunoblotting with the indicated antibodies or directly immunoblotted with the indicated antibodies as the input controls. (FIG. 6G) Immunoblotting analysis of IL-17RB K470R recruiting downstream effectors. IL-17RB-KO BxPC3 cells expressing the Flag-tagged WT, K333R, K454R and K470R of IL-17RB were treated with rIL-17B and the cell lysates were co-immunoprecipitated with anti-Flag-agarose and immunoblotted with the indicated antibodies or directly immunoblotted with the indicated antibodies as the input controls. (FIG. 6H to FIG. 6J) IL-17RB-knockout CFPAC1 cells expressing WT or each IL-17RB mutant with K333R, K454R, or K470R, were analyzed for their CCL20 and TFF1 mRNA expression (FIG. 6H) by RT-qPCR (n=3) after rIL-17B treatment. The same cells were assayed for SACF (n=4) (FIG. 6I) and invasion (n=4) (FIG. 6J).

FIGS. 7A to 7J include charts showing that disruption of the interaction between IL-17RB and MLK4 by the loop peptide blocks oncogenic progression. (FIG. 7A) Peptides TAT₄₈₋₅₇ (Control) (SEQ ID NO: 21) and TAT-IL-17RB₄₀₃₋₄₁₆ (Loop) (SEQ ID NO: 30) used for the following experiments. (FIG. 7B) Immunoblotting analysis of IL-17RB binding to MLK4 with Loop peptide treatment. 293T cells expressing Flag-MLK4 and HA-IL-17RB were pretreated with the control and loop peptides with different dose (0, 0.1, 1, 10, 50, 100 ng/ml) for 30 mins, and then added rIL-17B for 30 mins. The cell lysates were reciprocally co-immunoprecipitated with anti-Flag and anti-HA and blotted with the indicated antibodies or directly immunoblotted with the indicated antibodies as the input controls. (FIG. 7C to FIG. 7F) CFPAC1cells were pretreated with the peptides for 30 mins and then added rIL-17B for 30 mins. The cell lysates were immunoblotted with the indicated antibodies or immunoprecipitated with anti-IL-17RB or control IgG and blotted with the indicated antibodies (FIG. 7C). CFPAC1 cells were pretreated with TAT₄₈₋₅₇ or TAT-IL-17RB₄₀₃₋₄₁₆ (50 ng/ml) 30 mins and then added rIL-17B for two hrs. CCL20 and TFF/mRNA expression of these cells were measured by RT-qPCR (n=3) (FIG. 7D), the same cells were for invasion assay (n=4) (FIG. 7E) and SACF assay (n=4) (FIG. 7F). (FIG. 7G) The scheme of the treatment of transgenic pancreatic cancer EKP mice (LSL-Kras^{+/G12D}; p53^{+/-}; Ela-Cre^{ERT}; plus cerulein) with the Loop peptides. (FIG. 7H) IHC images of pancreases stained with anti-P-Y447 antibody. Scale bars: 50 μm. The pancreas tissues were from EKP mice after treated with the control or loop peptides at Day 42. The Kras^{+/+} mice (Kras^{+/+}; p53^{+/-}; Ela-Cre^{ERT}; plus cerulein) were used as the non-cancerous control. Boxes indicate the enlarged areas. (FIG. 7I) The experimental EKP mice (n=7 for each group) were euthanized at Day 56, and the metastatic tumor nodules in the lungs were counted. (FIG. 7J) Kaplan-Meier survival curve plot of the lifespans of the experimental EKP mice (n=7 for each group). Log-rank test was used.

FIGS. 8A to 8D include charts showing that knockout of IL-17RB diminishes IL-17B/IL-17RB oncogenic signaling. The IL-17RB-KO CFPAC1 and BxPC3 cells were established by the CRISPR/Cas9 system with two gRNA (#1 and #2). (FIG. 8A) Immunoblot analysis was used to assess IL-17RB and phospho-ERK1/2 expression in IL-17RB-KO cells treated with 50 ng/ml rIL-17B in serum-free conditions for 30 mins. RE: relative expression. (FIG. 8B to FIG. 8D) IL-17RB-KO cells were used for measuring the CCL20 and TFF1 mRNA expression (FIG. 8B) by RT-qPCR (n=3) after 50 ng/ml rIL-17B treatment in serum-free conditions for 2 hrs, for soft agar colony formation assay (SACF) (n=6) (FIG. 8C), or for invasion assay (n=6) (FIG. 8D) after supplementation with rIL-17B (50 ng/ml). Data in (FIG. 8B to FIG. 8D) are mean±s.d. *P<0.05 by two-tailed Student's t-test. **P<0.01 by two-tailed Student's t-test.

FIGS. 9A to 9D include charts showing that mutation of tyrosine 447 (Y447) of IL-17RB abrogates IL-17B-induced oncogenic signaling. (FIG. 9A) Sequence alignment of IL-17RB among different species (the full length sequences refer to SEQ ID NOs: 1 to 9, respectively). Arrow indicates Y447. The amino acid sequences of the full length of IL-17RB of different species are SEQ ID NOs: 1 to 9. (FIG. 9B to FIG. 9D) The effect of Y447 in IL-17RB oncogenic signaling. The wild-type (WT) and six tyrosine (Y)-to-phenylalanine (F) mutants of IL-17RB were ectopically expressed in IL-17RB-KO BxPC3 cells, separately, and the cell lysates were immunoblotted with the indicated antibodies (FIG. 9B). The same cells were for the measurement of the cytokine gene expression by RT-qPCR (n=3) (FIG. 9C), and for SACF assay (n=6) (FIG. 9D). Data in (FIG. 9C and FIG. 9D) are mean±s.d., **P<0.01 by two-tailed Student's t-test.

FIGS. 10A and 10B include charts showing that immunohistochemistry images of primary and metastatic pancreatic tumor specimens using anti-P-Y447 antibody. (FIG. 10A) Representative IHC images of pancreatic tumors with anti-P-Y447 and anti-IL-17RB (A81) antibodies. Low expression means <10% and high expression means >10% positive staining in cancer cell populations. Three serial sections were used for IHC. Boxes show enlarged regions. (FIG. 10B) Representative IHC images of liver metastatic pancreatic tumors with anti-P-Y447 antibody. Low expression means <10% and high expression means >10% positive staining in cancer cell populations. Scale bars: 50 μm.

FIGS. 11A to 11C include charts showing that depletion of AAK1, HIPK1 and Syk does not alter IL-17B-induced ERK1/2 phosphorylation. Immunoblotting analysis of cell lysates prepared from the AAK1- (FIG. 11A), HIPK1- (FIG. 11B) and Syk- (FIG. 11C) knockdown cells after 50 ng/ml rIL-17B treatment for 15 mins in serum-free conditions. The blots were probed with the indicated antibodies.

FIGS. 12A to 12D include charts showing that knockdown of MLK4 expression reduces IL-17B-induced ERK1/2 phosphorylation, CCL20 and TFF1 expression, and aggressive phenotypes of pancreatic and breast cancer cells. Pancreatic cancer cells (AsPC1 and BxPC3) and breast cancer cells (MB361 and MB468) were transduced with either lentiviral shLacZ (control) or lentiviral shMLK4, separately, and the cell lysates were immunoblotted directly with the indicated antibodies (FIG. 12A). The same cells were for RT-qPCR analysis of the cytokine gene expression (n=3) (FIG. 12B), for SACF assay (n=4) (FIG. 12C), and for invasion assay (n=4) (FIG. 12D). Data in (FIGS. 12B to 12D) are mean±s.d. *P<0.05 by two-tailed Student's t-test. **P<0.01 by two-tailed Student's t-test.

FIGS. 13A to 13G include charts showing that IL-17B, but not IL-17E, induces IL-17RB tyrosine phosphorylation, and FNmut mutant fails to homodimerization and transmits downstream oncogenic signaling. (FIG. 13A) Diagram illustrated the functional domain of IL-17RB. FN: fibronectin-III-like domain; SEFIR: similar expression to fibroblast growth factor genes and IL-17R, flexible loop: L395-E417 and Y447 and K470 in the wild-type and FNmut (FN2-truncated) of IL-17RB. Grey shade indicates as cell membrane. (FIG. 13B) WT and mutant IL-17RB-Flag was transfected to BxPC3 cells, separately. The membrane fractions and whole cell lysates were immunoblotted with the indicated antibodies. (FIG. 13C) Representative immunofluorescent images of the BxPC3 cells transfected with WT, FNmut, Y447F and K470R IL-17RB-Flag and stained with anti-Flag antibody. (FIG. 13D) Duolink in situ interaction assay for IL-17RB homodimerization. IL-17RB-KO BxPC3 cells were co-transfected with IL-17RB-HA and IL-17RB-Flag, or IL-17RB^{FNmut}-Flag, followed by treatment with 50 ng/ml rIL-17B or rIL-17E for 30 mins after serum starvation, and Duolink in situ interaction assay was performed using anti-HA and anti-Flag antibodies. Scale bars: 5 μm. (FIG. 13E and FIG. 13F) IL-17RB-KO BxPC3 cells were transfected with IL-17RB-Flag or IL-17RB^{FNmut}-Flag, and the cytokine gene expression of these cells were measured by RT-qPCR (n=3), (FIG. 13E) and for SACF assay (n=6) (FIG. 13F). Data in (FIG. 13E and FIG. 13F) are mean±s.d. *P<0.05 by two-tailed Student's t-test. **P<0.01 by two-tailed Student's t-test. (FIG. 13G) BxPC3 cells were treated with 50 ng/ml rIL-17B or rIL-17E, separately, in serum-free conditions for the indicated time. The cell lysates were immunoblotted directly with the indicated antibodies.

FIGS. 14A and 14B include charts showing that IL-17B-induced IL-17RB dimerization was not affected by knockdown of TRIM56, MLK4, or mutations of Y447 and K470 of IL-17RB. (FIG. 14A) IL-17RB-KO BxPC3 cells were transduced with shRNAs of TRIM56 or MLK4 to knockdown expressions of TRIM56 and MLK4, respectively. These cells were co-transfected with IL-17RB-HA and IL-17RB-Flag. After serum starvation, the cells were treated with rIL17B (50 ng/ml) for 30 mins and the cell lysates were immunoprecipitated with anti-HA-conjugated beads and blotted with the indicated antibodies as the input controls. (FIG. 14B) IL-17RB-HA was co-transfected with either Flag tagged WT, Y447F or K470R IL-17RB in IL-17RB-KO BxPC3 cells, separately. These cells were treated with rIL-17B (50 ng/ml) in a serum free condition for 15 mins and the cell lysates were immunoprecipitated with anti-HA-conjugated beads and immunoblotted with indicated the antibodies.

FIGS. 15A to 15D include charts showing that CEP-1347 inhibits IL-17RB Y447 phosphorylation, ERK1/2 phosphorylation, and aggressiveness of both pancreatic and breast cancer cells. Pancreatic cancer cells (AsPC1 and BxPC3) and breast cancer cells (MB361 and MB468) were transduced with lentiviral shLacZ (control) and lentiviral shMLK4, separately. In comparison to MLK4-knockdown cells, shLacZ-transduced cancer cells were pretreated with CEP-1347 (200 nM), a non-selective inhibitor of MLKs, before adding 50 ng/ml rIL-17B in serum-free conditions for 30 mins. The cell lysates were immunoblotted directly with the indicated antibodies (FIG. 15A). The same cells were measured by RT-qPCR for the cytokine gene expression (n=3) (FIG. 15B), for SACF assay (n=4) (FIG. 15C), and for invasion assay (n=4) (FIG. 15D). All the data are mean±s.d. *P<0.05, **P<0.01 by two-tailed Student's t-test.

FIGS. 16A and 16B show predicted structure of the SEFIR domain of human IL-17RB and the steric conformation of Y447. (FIG. 16A) Human IL-17RB SEFIR domain structure (blue) was predicted using mIl-17rb (3vbc, red) as a template, with Y447 indicated (green). Y444 of mIl-17rb is also shown (orange). (FIG. 16B) Y444 is located on the surface of the mouse Il-17rb SEFIR domain.

FIGS. 17A to 17F include charts showing that TRIM56 binds to N458˜V462 of IL-17RB and is critical for IL-17RB oncogenic signaling. (FIG. 17A) 293T cells were transfected with Flag-tagged WT, Y447F IL-17RB and the cell lysates were immunoprecipitated with M2 beads and immunoblotted with anti-P-Y447 and anti-flag antibodies (upper). The WT and Y447F Flag-IL-17RB were purified from these cells by anti-FLAG (M2) beads, while HA-TIM56 expressing in 293T cells was purified by anti-HA beads, respectively. The Flag-IL-17RB (WT or Y447F) proteins were mixed with HA-TRIM56 and incubated on ice for 30 mins. The protein mixtures were then separated by native-PAGE followed by Coomassie blue staining (bottom). (FIG. 17B to FIG. 17E) Pancreatic cancer cells (CFPAC1, AsPC1 and BxPC3) and breast cancer cells (MB361 and MB468) were transduced with lentiviral shLacZ (control) and lentiviral shTRIM56, separately, and the cell lysates were immunoblotted with the indicated antibodies (FIG. 17B and see also FIG. 6C), These cells were for RT-qPCR analysis of the cytokine gene expression (n=3) (FIG. 17C), for SACF) assay (n=4) (FIG. 17D), and for invasion assay (n=4) (FIG. 17E). *P<0.05 by two-tailed Student's t-test. Data in (FIG. 17C to FIG. 17E) are mean±s. d. **P<0.01 by two-tailed Student's t-test. (FIG. 17F) The Flag-tagged WT or Del-6 (ΔN458—V462) IL-17RB were transduced into SU.86.86 cells, separately. After 50 ng/ml rIL-17B treatment in serum-free conditions for 30 min, co-IP with anti-Flag-conjugated beads was performed and followed by immunoblotting analysis with the indicated antibodies.

FIGS. 18A to 18E include charts showing that TRIM56 serves as an E3 ligase for K63-linked ubiquitination of IL-17RB. (FIG. 18A) Flag-IL-17RB was co-transfected with HA-tagged WT and mutants of Ub into 293T cells, separately. These cells were then treated with 50 ng/ml rIL-17B in serum-free conditions for 30 mins. Whole cell lysates were collected for co-immunoprecipitation with anti-HA-agarose and immunoblotted with the indicated antibodies. (FIG. 18B) Diagram illustrated the three lysine residues (K333, K454 and K470, blue) and Y447 (red) on the surface of the IL-17RB intracellular domain. (FIG. 18C) Flag-tagged WT, K333F, K454F and K470F of IL-17RB constructs were transfected into IL-17RB-KO BxPC3 cells. These cells were treated with 50 ng/ml rIL-17B in serum-free conditions for 30 mins. Whole cell lysates were collected for co-immunoprecipitation with anti-Flag-agarose and immunoblotting analysis with the indicated antibodies. (FIG. 18D) Affinity-purified Flag-IL-17RB (WT or Y470F) protein and HA-TRIM56 (WT or Δ31-50) proteins were used for in vitro ubiquitination assay with the recombinant E1, E2 and ubiquitin. The protein mixtures were then separated by SDS-PAGE followed by immunoblotting analysis with anti-K48-Ub and anti-K63-Ub (upper) and Coomassie blue staining (bottom). (FIG. 18E) Act-knockdown, TRAF6-knockout and control AsPC1 cells were treated with 50 ng/ml rIL-17B and the cell lysates were co-immunoprecipitated anti-IL-17RB (D9)-conjugated beads and immunoblotted with the indicated antibodies.

FIGS. 19A to 19D include charts showing that treatment with the loop peptide suppresses aggressive behavior of patient-derived pancreatic tumor cells. (FIG. 19A) CFPAC1 cells were treated with Alexa Fluor 568-labeled or non-labeled cold peptides of TAT₄₈₋₅₇ (Control) and TAT-IL-17R₄₀₃₋₄₁₆ (Loop) for 30 mins, respectively. Confocal microscopy was used for visualization of peptide penetration into cells. (FIG. 19B) The PDX-derived tumor cells (PC080 left, PC084 right) were pretreated with the peptides for 30 mins and then treated with 50 ng/ml rIL-17B for 2 hrs in serum-free conditions. (FIG. 19C and FIG. 19D) These cells were measured by RT-qPCR for the cytokine gene mRNA expression (n=3), or for Invasion assay (n=4) (FIG. 19C) and SACF assay (n=4) (FIG. 19D). Data in (FIG. 19B—FIG. 19D) are mean±s.d. * P<0.05, **P<0.001 by two-tailed Student's t-test.

FIGS. 20A to 20D include charts showing that treatment of TAT-IL-17RB₄₀₃₋₄₁₆ peptide reduces the recruitment of MDSC and M2 macrophage. (FIG. 20A) Pancreatic tissues of EKP mice (LSL-Kras+/G12D; p53+/−; Ela-Cre^{ERT}; plus cerulein) (n=7 for each group) treated with TAT-IL-17RB₄₀₃₋₄₁₆ (Loop) peptides were examined the tumor-immune modulating capacity by flow cytometry with specific cell surface markers as indicated. (FIG. 20B to FIG. 20D) Representative dot plots revealed the presence of F4/80 and CD206 in CD45+ immune cells (upper), and CD11b+/Gr1+ MDSC (bottom). The plots showed the quantitation of percentage of CD11b+/Gr1+ MDSC (FIG. 20B), F4/80+ macrophage in CD45+ cells (FIG. 20C), and CD206+M2 macrophage in CD45+/F4/80+ cells (FIG. 20D). Data are mean±s.d. *P<0.05, **P<0.01 by two-tailed Student's t-test.

FIGS. 21A to 21G include charts showing that disruption of IL-17RB and MLK4 interaction by the loop peptide suppresses pancreatic tumor progression. (FIG. 21A) The diagram illustrated the schedule and timeline of the mouse xenograft experiments testing the anti-tumor effect of the peptides in an orthotopically xenografted pancreatic cancer mouse model. About 2×10⁵ CFPAC1-GFP/Luc cells were transplanted into the pancreases of the NOD-SCID mice for seven days. PBS, control and loop peptides (160 μg/100 μl PBS, twice per week, n=7 for each group) were intraperitoneally injected, separately. (FIG. 21B) Representative IHC images of P-Y447 in pancreases from tumor-bearing mice after peptide treatment. Boxes show enlarged areas. Scale bars: 50 μm. (FIG. 21C and FIG. 21D) Tumor growth in the mice at the indicated times was monitored by IVIS imaging system after pancreatic cancer cell (CFPAC1-GFP/Luc) implantation (FIG. 21C). Tumor growth curves were plotted by the intensity of the bioluminescent signals from IVIS imaging (FIG. 21D). Data are mean±s.d. *P<0.05, **P<0.01 by two-tailed Student's t-test. (FIG. 21E) Kaplan-Meier survival analysis of the pancreatic tumor-bearing mice after treatment with the peptides. Log-rank test was used. (FIG. 21F) Lung and liver metastasis of the pancreatic tumors after treatment with the peptides. At Day 34, the tumor-bearing mice (n=5 each group) were euthanized and livers and lungs were collected to examine the distant metastases of the pancreatic cancer cells. The metastatic pancreatic tumors in the livers and lungs of the peptide-treated mice were revealed by IVIS imaging. (FIG. 21G) Plots of the quantification results of bioluminescent signals in liver and lung metastatic tumors. Data are mean±s.d. *P<0.05, **P<0.01 by two-tailed Student's t-test.

FIGS. 22A to 22D include charts showing that overexpression of IL-17RA or treating with IL-17E reduces IL-17B-mediated IL-17RB dimerization and tyrosine phosphorylation. (FIG. 22A and FIG. 22B) IL-17RB-HA, IL-17RB-Flag and IL-17RA-HA were co-transfected into IL-17RB-KO BxPC3 cells. These cells were treated with rIL17B, rIL17E or both in a serum free condition for 15 mins. Cell lysates were co-immunoprecipitated with anti-HA-conjugated beads and blotted with the indicated antibodies (FIG. 22A), and quantification result was shown with a bar chart (FIG. 22B). (FIG. 22C and FIG. 22D) BxPC3 cells alone or expressing IL-17RA were cotreated with rIL-17B (50 ng/ml) and the indicated amount of IL-17E in a serum free condition for 30 mins. Cell lysates were immunoblotted with the indicated antibodies (FIG. 22C) and quantification result was shown with a bar chart (FIG. 22D).

FIG. 23 shows graphical summary: strategies for targeting key steps of IL-17RB/B-driven oncogenic signaling. Binding of IL-17B specifically induces IL-17RB homo-dimerization, leading to the recruitment of MLK4 and subsequent phosphorylation of IL-17RB at Y447. P-Y447 is recognized and bound by TRIM56 for K63-linked poly-ubiquitination at K470 of IL-17RB (Ub-K470), which initiates downstream oncogenic signaling. In turn, interruption of the interaction between IL-17RB and MLK4 by loop peptide (TAT-IL-17RB₄₀₃₋₄₁₆) inhibits activation of IL-17B/IL-17RB oncogenic signaling to suppress pancreatic tumor growth and metastasis.

FIGS. 24A to 24G include charts showing that loop peptide does not suppress IL-17E-induced IL-4 and IL-13 mRNA expression in PBMCs or affect bone marrow-derived dendritic cell activation. (FIG. 24A) Flow chart describing the experimental design for testing the effects of anti-IL-17RB antibody and loop peptide on IL-17E-induced expression of IL-4 and IL-13 in peripheral blood mononuclear cells (PBMCs). PBMCs were purified by Ficoll-paque and used for the following experiments. (FIG. 24B to FIG. 24E) IL-4 and IL-13 expression induced by IL-17B or IL-17E in the PBMCs was determined by RT-qPCR after mIgG or anti-IL-17RB antibody pretreatment (FIG. 24B and FIG. 24C) and control or loop peptide pretreatment (FIG. 24D and FIG. 24E), respectively. (FIG. 24F and FIG. 24G) The DCs derived from mouse bone marrow were treated with the control or loop peptides, separately. (FIG. 24F) Flow cytometry analysis using anti-CD80, CD86 and MHC-II antibodies was performed to determine the activity of the DCs after incubation with the control or loop peptides for 48 hrs. (FIG. 24G) The activities of DCs were quantified by gating the area of the CD80, CD86 and MHC-II-positive cells (n=3). Data are mean±s.d.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “polypeptide” refers to a polymer composed of amino acid residues linked via peptide bonds. The term “peptide” refers to a relatively short polypeptide composed of linked amino acids e.g., 200 amino acids or less, 175 amino acids or less, 150 amino acids or less e.g. 140 or less, 130 or less, 120 or less, 110 or less, 100 or less, 90 or less, 80 or less, 70 or less, 60 or less, 50 or less or 40 or less amino acids in length.

As used herein, “corresponding to,” refers to a residue at the enumerated position in a protein or peptide, or a residue that is analogous, homologous, or equivalent to an enumerated residue in a protein or peptide.

As used herein, the term “fusion protein” refers to a protein produced by genetic technology, which comprises two or more functional domains derived from different proteins. The fusion protein can be prepared in a conventional manner, for example, by gene expression of the nucleotide sequence encoding the fusion protein in a suitable cell.

IL-17RB is one of the IL-17 receptors which are single-pass transmembrane proteins. IL-17RB as described herein can include human IL-17RB and its homologues from vertebrates, and particularly those homologues from mammals. Specifically, IL-17RB as described herein includes the IL-17RB amino acid sequences from human (SEQ ID NO: 1), and the IL-17RB amino acid sequences from other mammals (SEQ ID NOs: 2 to 9). IL-17RB as described herein further includes any recombinantly (engineered)-derived IL-17RB polypeptides encoded by cDNA copies of the natural polynucleotide sequence encoding IL-17RB.

In structure, IL-17RB includes an extracellular domain, a transmembrane domain and an intracellular cytoplasmic tail. Specifically, the extracellular domain is located at positions corresponding to positions 18-289 of SEQ ID NO: 1, the transmembrane domain is located at positions corresponding to positions 290-312 of SEQ ID NO: 1, the intracellular cytoplasmic tail is located at positions corresponding to positions 313-502 of SEQ ID NO: 1, in which a flexible loop for MLK4 binding is located at positions 403-416.

Below shows the amino acid sequence of IL17RB from human and other mammals (SEQ ID NOs: 1 to 9).

IL-17RB; interleukin-17 receptor B [Homo sapiens (human)]
NCBI-GeneID: 55540
Source: www.genome.jp/dbget-bin/www_bget?hsa:55540
(SEQ ID NO: 1)
Amino Acid Sequence (502 aa)
MSLVLLSLAALCRSAVPREPTVQCGSETGPSPEWMLQHDLIPGDLRDLRVEPVTTSVATG
DYSILMNVSWVLRADASIRLLKATKICVTGKSNFQSYSCVRCNYTEAFQTQTRPSGGKWT
FSYIGFPVELNTVYFIGAHNIPNANMNEDGPSMSVNFTSPGCLDHIMKYKKKCVKAGSLW
DPNITACKKNEETVEVNFTTTPLGNRYMALIQHSTIIGFSQVFEPHQKKQTRASVVIPVT
GDSEGATVOLTPYFPTCGSDCIRHKGTVVLCPQTGVPFPLDNNKSKPGGWLPLLLLSLLV
ATWVLVAGIYLMWRHERIKKTSFSTTTLLPPIKVLVVYPSEICFHHTICYFTEFLQNHCR

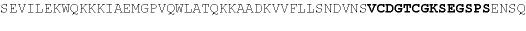

DLFPLAFNLFCSDLRSQIHLHKYVVVYFREIDTKDDYNALSVCPKYHLMKDATAFCAELL
HVKQQVSAGKRSQACHDGCCSL
Note: The area with gray background indicates the flexible loop
for MLK4 binding (aa 403-416)
IL-17rb; interleukin-17 receptor B [Pan troglodytes
(Chimpanzee))]
NCBI-GeneID: 460451
Source: https://www.ncbi.nlm.nih.gov/gene/460451
(SEQ ID NO: 2)
Amino Acid Sequence (505 aa)
MSLVLLSLAALCRSAVPREPTVQCGSETGPSPEWMLQHDLIPGDLRDLRVELVTTSVATG
DYSILMNVSWVLRADASIRLLKATKICVTGKSNFQSYSCVRCNYTEAFQTQTRPSGGKWT
FSYVGFPVELNTVYFIGAHNIPNANMNEDGPSMPVNFTSPGCLDHIMKYKKKCVKAGSLW
DPNITACKKNEETVEVNFTTTPLGNRYMALIQHSTHSTVIGESQVFEPHQKKQTRASVVI
PVTGDSEGAMVOLTPYFPTCGSDCIRHKGIVVLCPQTGVPFPLDNNKSKLGGWLPLLLLS
LLVATWVLVAGIYLMWRHERIKKTSFSTTTLLPPIKVLVVYPSEICFHHTICYFTEFLON

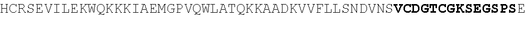

NSQDLFPLAFNLFCSDLRSQIHLHKYVVVYFRETDTKDDYNALSVCPKYHLMKDATAFCA
ELLHVKQQVSAGKRSQACHDGCCSL
Note: The area with gray background indicates the flexible loop
for MLK4 binding (aa 406-419)
IL-17rb; interleukin-17 receptor B [Gorilla (Western lowland
gorilla)]
NCBI-GeneID: 101142225
Source: https://www.ncbi.nlm.nih.gov/gene/101142225
(SEQ ID NO: 3)
Amino Acid Sequence (502 aa)
MSLVLLSLAALCRSAVPREPTIQCGSETGPSPEWMLQHDLIPGDLRDLRVEPVKTSVATG
DYSILMNVSWVLRADASIRLLKATKICVTGKSNFQSYSCVRCNYTEAFQTQTRPSGGKWT
FSYIGFPVELNTVYFIGAHNIPNANMNEDGPSMSVNFTSPGCLDHIMKYTKKCVKAGSLW
DPNITACKKNEETVEVNETTTPLGNRYMALIQHSTIIGFSQVFEPHQKKOTRASVVIPVT
GDSEGAMVOLTPYFPTCGSDCIRHKGTVVLCPQTGVPFPLDNNKSKPGGWLPLLLLSLLV
ATWVLVAGIYLMWRHERIKKTSFSTTTLLPPIKVLVVYPSEICFHHTICYFTEFLQNHCR

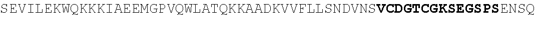

DLFPLAFNLFCSDLRSQIHLHKYVVVYFRETDTKDDYNALSVCPKYHLMKDATAFCAELL
HVKQQVSAGKRSQACHDGCCSL
Note: The area with gray background indicates the flexible loop
for MLK4 binding (aa 403-416)
IL-17rb; interleukin-17 receptor B [Pteropus alecto (Black
flying fox)]
NCBI-GeneID: 102891713
Source: https://www.ncbi.nlm.nih.gov/gene/ 102891713
(SEQ ID NO: 4)
Amino Acid Sequence (454 aa)
MGGDDLAMSLMLLSLAALCWGAVSPEPTIQCGPETGPSPEWMVRHTLTPGDLRDLRVEPV
KSSVASEDYSILMNISWILRADASIRLLKATKICVTGKSGFQSYGCVRCNYTEVFOTQSR
PSGGKWMFFYIGFPVELNTLYFIGAHNIPNANMNEDSPSMSVNFTSPGSLWDPNITACKK
NENMVEVNFTISPLGNRYMVLILKNTVIGTSVSEEKLTRTSVMVPVTGESEGAVVOLTPY
FHTCGNDCIRRKGMVVLCPQTGVPFSPDNKKIKMSLSSTMLLPITVLVVYPSEICFHHTV

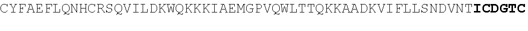

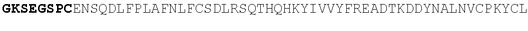

MKDATSFCMELLHVEQQVSTGKRLRACHNRCSSL
Note: The area with gray background indicates the flexible loop
for MLK4 binding (aa 355-368)
IL-17rb; interleukin-17 receptor B [Rattus norvegicus (Rat)]
NCBI-GeneID: 306247
Source: https://www.ncbi.nlm.nih.gov/gene/306247
(SEQ ID NO: 5)
Amino Acid Sequence (354 aa)
MNEDSPSLSVNFTSPGCLNHVMKYKKQCIEAGSLWDPNITACKKNEKTVEVNFTTNSLGN
RYMVLIRRDTMLGVSIVLENKLTRTSVVIPVNDESEGALVELTPYLHTCDNDCIRRKGTV
VLCSETSAPFPPDDNRSMLRGWLPLLLVLLVATWVLAVGIYLTWRQGRSTKTSFPITAML
LPLVKVLVVYPSEICFHHTVCRFTDFLQNYCRSEVILEKWQKKKIAEMGPVQWLTTQKQA

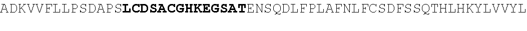

GGADLKGDYNALRVCPQYHLMKDAPAFHTELLKATQSMPLKKRPQACHGSCSPL
Note: The area with gray background indicates the flexible loop
for MLK4 binding (aa 255-268)
IL-17rb; interleukin-17 receptor B [Mus musculus (Mouse)]
NCBI-GeneID: 50905
Source: https://www.ncbi.nlm.nih.gov/gene/50905
(SEQ ID NO: 6)
Amino Acid Sequence (499 aa)
MLLVLLILAASCRSALPREPTIQCGSETGPSPEWMVQHTLTPGDLRDLQVELVKTSVAAE
EFSILMNISWILRADASIRLLKATKICVSGKNNMNSYSCVRCNYTEAFQSQTRPSGGKWT
FSYVGFPVELSTLYLISAHNIPNANMNEDSPSLSVNFTSPGCLNHVMKYKKQCTEAGSLW
DPDITACKKNEKMVEVNETTNPLGNRYTILIQRDTTLGESRVLENKLMRTSVAIPVTEES
EGAVVQLTPYLHTCGNDCIRREGTVVLCSETSAPIPPDDNRRMLGGWLPLFLVLLVAVWV
LAAGIYLTWRQGRSTKTSFPISTMLLPLIKVLVVYPSEICFHHTVCRFTDELQNYCRSEV

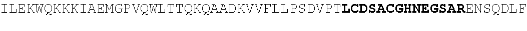

PLAFNLFCSDESSQTHLHKYLVVYLGGADLKGDYNALSVCPQYHLMKDATAFHTELLKAT
QSMSVKKRSQACHDSCSPL
Note: The area with gray background indicates the flexible loop
for MLK4 binding (aa 400-413)
IL-17RB; interleukin-17 receptor B [Equus caballus (Horse)]
NCBI-GeneID: 100059421
Source: https://www.ncbi.nlm.nih.gov/gene/100059421
(SEQ ID NO: 7)
Amino Acid Sequence (499 aa)
MSLVLLSLAALCWGAVPREPTIQCGSEAGPSPEWMVQHALTPGDLRDLQVEPVKSRVATE
DYSVLMNISWILRADASIRFLKATKICVTGKSNFQSYSCVRCNYTEAFQTQTRPSGGKWT
FSYIGFPVELDTLYFIGAHNIPNANMNGDGPSLSVNFTSPGCLDHVMKYKKKCIEAGSLW
DPNITACKKNEKMVEVNFTTSPLGNRYMALIQNNTVIGLSNVLENKLTRTSVVIPVTGES
EGAVVOLTPYFHTCGSDCIRRRGIVVLCPHTGASSPPDNSRSVLGGWLPFLLPALLVATW
VLAVGIYLTWRHERIKKTSFPTTALLSPIKVLVVYPSEICFHHTVCYFTKFLQNHCRTEV

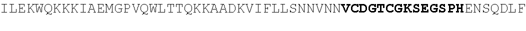

PLAFNLFCSDLRSQNHLHKYMVVYFREADTKDDYDALHVCPKYCLMKDAAAFCTELLHVE
QHVSVGKRWRACHNRCSAL
Note: The area with gray background indicates the flexible loop
for MLK4 binding (aa 400-413)
IL-17rb; interleukin-17 receptor B [Sus scrofa (Pig)]
NCBI-GeneID: 100156014
Source: https://www.ncbi.nlm.nih.gov/gene/100156014
(SEQ ID NO: 8)
Amino Acid Sequence (477 aa)
MLLVLLSLAALCWGAMPPEPTIQCGSEPGLSPEWMVRHALTPGDLRDLRVEPIKSSVAVE
DYSILMNISWILRADASIRLLKATKICVTGKSQKQTYSCVRCNYTEAFQTQTRPSGGKWM
FSYVGFPVELNTRYFIGAHNIPNANMNEDGPSLAVNFTSPGCLDRIMKYKKKCIEAGSLW
DPNITACKKSENTVEVNFTTSPLGNRYMALIQNSTVIGTSYVSELTPYFRTCGNDCIRRR
GTVVRCPHTGVPFPQDQSRSMLSGWLPLLLLALLVAIWVLAGGIYLTRRHERIKKTSESA
TILLPPIKVLVVYPSEICFHHTVCYFTRFLQNHCRSEVILEKWQKKKIAEMGPVOWLTTO

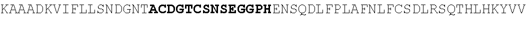

VYFREVDIKDDYSALSVCPTYHLMKDAPAFCKELLHAEQHVSVGRRLQACHYSCSSL
Note: The area with gray background indicates the flexible loop
for MLK4 binding (aa 378-391)
IL-17rb; interleukin-17 receptor B [Felis catus (Cat) (Felis
silvestris catus)]
NCBI-GeneID: 101080437,
Source: https://www.ncbi.nlm.nih.gov/gene/101080437
(SEQ ID NO: 9)
Amino Acid Sequence (494 aa)
MSLALLSLAALCWGLVSTEPTIQCGSEPGPSPEWMVQHTLTPGDLRDLRVEPVRSRVAMD
YSILMNVSWVLRADASIRLLKATKICVTGKSNLQSYSCVRCNYTEAFQTQTRPSGGRWTF
SYVGFPVELNTVYFIGAHNIPNANINEDSPSMSVNFTSPGCLDHIMKYQKKCIKAGSLWD
PNVTACKKNKTVVEVNFTTSPLGNKYMALIQNRIVIGESNVLENKPPTRTSVVIPVTGES
EGAMVOLTPYFHTCGNDCIRRKGTVVLCPQTGISFPLDGRRSMMGGWQPFLLPALLGATA
LLAAGIYVIWRHKRIKKASFPTATLLSPIKVLVVYPSEICFHHTVCHFTEFLONHCRSEV

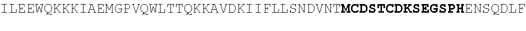

PLAFNLFCSDLRNQTPLRKYMVVYFREADTKDEYSALSVCPKYRLMKDAPAFCTELLRVE
QHMSAGKRLTACSM
Note: The area with gray background indicates the flexible loop
for MLK4 binding (aa 400-413)

In function, IL-17RB can be activated upon stimulation by the cytokine IL-17B. IL-17RB forms a homo-dimer upon IL-17B binding and recruits MLK4 through the flexible loop to phosphorylate it at position Y447, and the phosphorylated IL-17B in turn recruits TRIM56, an ubiquitin ligase, to add K63-linked ubiquitin chains on position K470. Activation of the IL-17B/IL-17RB signaling confers oncogenic activities. IL-17RB also can be recognized by IL-17E. However, unlike IL-17B, the binding of IL-17E to IL-17RB induces hetero-dimerization of IL-17RB with IL-17RA to activate Th2 immune responses which is not mediated by MLK4 phosphorylation.

The present disclosure is based, at least in part, on the finding that any of the events including the IL-17B/IL-17RB signaling through MLK4 phosphorylation at Y447 and TRIM56 ubiquitination at K470 is critical for oncogenesis. Accordingly, provided herein are methods for inhibiting IL-17B/IL-17RB activation and thus treating a disease or disorder associated therewith with an IL-17RB antagonist which targets the interaction between IL-17RB and MLK4, Y447 phosphorylation and/or K470 ubiquitination. Especially, the methods described herein would induce no certain side effects, such as reduction of type 2 immunity.

As used herein, the term “IL-17RB antagonist” refers to a substance or an agent which can substantially reduce, inhibit, block and/or mitigate activation of IL-17RB signaling, particularly IL-17B/IL-17RB signaling. In some embodiments, the IL-17RB antagonist as used herein are capable of inhibiting the interaction between IL-17RB and MLK4, for example, by competing the binding site of MLK4 in IL-17RB, thus substantially reducing, inhibiting, blocking and/or mitigating activation of IL-17RB. The IL-17RB antagonist for use in the present invention may include a peptide or a small molecular compound. Specifically, the IL-17RB antagonist for use in the present invention does not inhibit IL-17RB immunogenic signaling through IL-17E.

In some embodiments, the present invention discloses an IL-17RB inhibitory peptide acting as an IL-17RB antagonist for inhibiting IL-17B/IL-17RB activation. The IL-17RB inhibitory peptide is a non-naturally occurring fragment including the amino acid sequences of the loop for MLK4 binding of IL-17RB. Such peptide competes the binding site of MLK4 in IL-17RB and is useful in suppressing the IL-17B/IL-17RB medicated oncogenic signaling pathway and thus benefits treatment of diseases and disorders associated with abnormal IL-17RB activation.

In some embodiments, the IL-17RB inhibitory peptide comprises a first segment that comprises the motif of X₁CDX₂X₃CX₄X₅X₆EGX₇X₈X₉ (SEQ ID NO: 10), wherein X₁ is valine (V), isoleucine (I), leucine (L), alanine (A), methionine (M). X₂ is glycine (G) or serine (S), X₃ is threonine (T) or alanine (A), X₄ is glycine (G), serine (S) or aspartic acid (D), X₅ is lysine (K), histidine (H) or asparagine (N), X₆ is serine (S), lysine (K) or asparagine (N), X₇ is serine (S) or glycine (G), X₈ is proline (P) or alanine (A), and X₉ is serine (S), cysteine (C), Threonine (T), arginine (R) or histidine (H).

In some embodiments, the first segment comprises the motif of X₁CDX₂X₃CGX₅X₆EGSX₈X₉ (SEQ ID NO: 11), wherein Xi is valine (V), isoleucine (I) or leucine (L), X₂ is glycine (G) or serine (S), X₃ is threonine (T) or alanine (A), X₅ is lysine (K) or histidine (H), X₆ is serine (S), lysine (K) or asparagine (N), X₈ is proline (P) or alanine (A), and X₉ is serine (S), cysteine (C), Threonine (T), arginine (R) or histidine (H).

In some embodiments, the first segment comprises the motif of X₁CDGTCGKSEGSPX₉ (SEQ ID NO: 12), wherein Xi is valine (V) or isoleucine (I), and X₉ is serine (S), cysteine (C) or histidine (H).

In some embodiments, the first segment comprises the motif of VCDGTCGKSEGSPX₉ (SEQ ID NO: 13), wherein X₉ is serine (S) or histidine (H).

In particular examples, the first segment comprises the amino acid sequence selected from the group consisting of SEQ ID Nos: 14-20.

| | | | |
|---|---|---|---|
| VCDGTCG | SEQ ID | human | aa 403-416 of SEQ ID |
| KSEGSPS | NO: 14 | | NO: 1 |
| VCDGTCG | SEQ ID | chimpanzee | aa 406-419 of SEQ ID |
| KSEGSPS | NO: 14 | | NO: 2 |
| VCDGTCG | SEQ ID | gorilla | aa 403-416 of SEQ ID |
| KSEGSPS | NO: 14 | | NO: 3 |
| ICDGTCG | SEQ ID | fox | aa 355-368 of SEQ ID |
| KSEGSPC | NO: 15 | | NO: 4 |
| LCDSACG | SEQ ID | rat | aa 255-268 of SEQ ID |
| HKEGSAT | NO: 16 | | NO: 5 |
| LCDSACG | SEQ ID | mouse | aa 400-413 of SEQ ID |
| HNEGSAR | NO: 17 | | NO: 6 |
| VCDGTCG | SEQ ID | horse | aa 400-413 of SEQ ID |
| KSEGSPH | NO: 18 | | NO: 7 |
| ACDGTCS | SEQ ID | pig | aa 378-391 of SEQ ID |
| NSEGGPH | NO: 19 | | NO: 8 |
| MCDSTCD | SEQ ID | cat | aa 400-413 of SEQ ID |
| KSEGSPH | NO: 20 | | NO: 9 |

In particular examples, the first segment comprises the amino acid sequence selected from the group consisting of SEO ID Nos: 14-18.

| | | | |
|---|---|---|---|
| VCDGTCG | SEQ ID | human | aa 403-416 of SEQ ID |
| KSEGSPS | NO: 14 | | NO: 1 |
| VCDGTCG | SEQ ID | chimpanzee | aa 406-419 of SEQ ID |
| KSEGSPS | NO: 14 | | NO: 2 |
| VCDGTCG | SEQ ID | gorilla | aa 403-416 of SEQ ID |
| KSEGSPS | NO: 14 | | NO: 3 |
| ICDGTCG | SEQ ID | fox | aa 355-368 of SEQ ID |
| KSEGSPC | NO: 15 | | NO: 4 |
| LCDSACG | SEQ ID | rat | aa 255-268 of SEQ ID |
| HKEGSAT | NO: 16 | | NO: 5 |
| LCDSACG | SEQ ID | mouse | aa 400-413 of SEQ ID |
| HNEGSAR | NO: 17 | | NO: 6 |
| VCDGTCG | SEQ ID | horse | aa 400-413 of SEQ ID |
| KSEGSPH | NO: 18 | | NO: 7 |

In particular examples, the first segment comprises the amino acid sequence selected from the group consisting of SEQ ID Nos: 14, 15 and 18.

| | | | |
|---|---|---|---|
| VCDGTCG | SEQ ID | human | aa 403-416 of SEQ ID |
| KSEGSPS | NO: 14 | | NO: 1 |
| VCDGTCG | SEQ ID | chimpanzee | aa 406-419 of SEQ ID |
| KSEGSPS | NO: 14 | | NO: 2 |
| VCDGTCG | SEQ ID | gorilla | aa 403-416 of SEQ ID |
| KSEGSPS | NO: 14 | | NO: 3 |
| ICDGTCG | SEQ ID | fox | aa 355-368 of SEQ ID |
| KSEGSPC | NO: 15 | | NO: 4 |
| VCDGTCG | SEQ ID | horse | aa 400-413 of SEQ ID |
| KSEGSPH | NO: 18 | | NO: 7 |

In particular examples, the first segment comprises the amino acid sequence selected from the group consisting of SEQ ID Nos: 14 and 18.

| | | | |
|---|---|---|---|
| VCDGTCG | SEQ ID | human | aa 403-416 of SEQ ID |
| KSEGSPS | NO: 14 | | NO: 1 |
| VCDGTCG | SEQ ID | chimpanzee | aa 406-419 of SEQ ID |
| KSEGSPS | NO: 14 | | NO: 2 |
| VCDGTCG | SEQ ID | gorilla | aa 403-416 of SEQ ID |
| KSEGSPS | NO: 14 | | NO: 3 |
| VCDGTCG | SEQ ID | horse | aa 400-413 of SEQ ID |
| KSEGSPH | NO: 18 | | NO: 7 |

In additional embodiments, the IL-17RB inhibitory peptide as described herein may be a variant thereof with one or more mutations. It is understandable that a polypeptide may have a limited number of changes or modifications that may be made within a certain portion of the polypeptide irrelevant to its activity or function and still result in a functionally equivalent variant with an acceptable level of equivalent or similar biological activity or function. In some examples, the amino acid residue mutations are conservative amino acid substitution, which refers to the amino acid residue of a similar chemical structure to another amino acid residue and the polypeptide function, activity or other biological effect on the properties smaller or substantially no effect. Variants can be prepared according to methods for altering polypeptide sequence known to one of ordinary skills in the art such as those found in references which compile such methods, e.g. Molecular Cloning: A Laboratory Manual, J. Sambrook, et al., eds., Second Edition, Cold Spring Harbor Laboratory Press, Cold Spring Harbor, New York, 1989. For example, conservative substitutions of amino acids include substitutions made amongst amino acids within the following groups: (i) A, G; (ii) T, S; (iii) Q, N; (iv) D, E; (v) M, I, L, V; (vi) F, Y, W; and (vii) K, R, H.

As used herein, the term “substantially identical” refers to two sequences having 70% or more, preferably 75% or more, more preferably 80% or more, even more preferably 85% or more, still even more preferably 90% or more, and most preferably 95% or more or 100% identity.

To determine the percent identity of two sequences, the sequences are aligned for optimal comparison purposes (e.g., gaps can be introduced in the sequence of a first nucleotide sequence for optimal alignment with a second nucleotide sequence). In calculating percent identity, typically exact matches are counted. The determination of percent homology or identity between two sequences can be accomplished using a mathematical algorithm known in the art, such as BLAST and Gapped BLAST programs, the NBLAST and XBLAST programs, or the ALIGN program.

In some embodiments, the IL-17RB inhibitory peptide as described herein may also include a substantially identical amino acid sequence to the particular sequence no. as described herein e.g. an amino acid sequence having 70% or more, preferably 75% or more, more preferably 80% or more, even more preferably 85% or more, still even more preferably 90% or more, and most preferably 95% or more or 100% identity to SEQ ID NO: 14, 15, 16, 17, 18, 19 or 20.

In some embodiment, the IL-17RB inhibitory peptide of the present invention further includes a second segment that comprises a cell-penetrating peptide sequence which is fused to the first segment.

As used herein, a cell-penetrating peptide sequence is described with respect to a peptide chain that directs a polypeptide transport within the cell. The delivery process into the cell may occur via endocytosis while the peptide may also be internalized into the cell through direct membrane translocation. The amino acid composition of a cell-penetrating peptide usually contains high relative abundance of positively charged amino acids (such as lysine (L) or arginine (R)), or has a sequence containing alternating patterns of polar/charged amino acids and non-polar hydrophobic amino acids. In one embodiment, the cell penetrating peptide sequence is fused at the N-terminus of the first segment as described herein. In another embodiment, the cell penetrating peptide is fused at the C-terminus of the first segment as described herein.

Examples of a cell-penetrating peptide sequence include SEQ ID NO: 21-29.

| | |
|---|---|
| (HIV-TAT) | RKKREQRRR (SEQ ID NO: 21) |
| Penetratin | RQIKIWPQNRRMKWKK (SEQ ID NO: 22) |
| SAP | VRLPPPVRLPPPVRLPPP(SEQ ID NO: 23) |
| HIV-1 Rev | TRQARRNRRRRWRERQR(SEQ ID NO: 24) |
| FHV | RRRRNRTRRNRRRVR (SEQ ID NO: 25) |
| HITIL-II | TRRQRTRRARRNR (SEQ ID NO: 26) |
| NLS | KRPAAIKKAGQAKKKK (SEQ ID NO: 27) |
| Transportan | GWTINSAGYLLGKINLKALAALAKKIL |
| | (SEQ ID NO: 28) |
| pVEC | LLIILRRRIRKQAHAHSK (SEQ ID NO: 29) |

In one particular example, the IL-17RB inhibitory peptide of the present invention comprises or consists of the amino acid sequence as set forth in PKKRRQRRRVCDGTCGKSEGSPS (SEQ ID NO: 30).

In some embodiments, the cell penetrating peptide is fused with the first segment via a flexible peptide, spacer peptide or linker peptide which does not substantially affect the IL-17RB inhibitory activity of the first segment and the cell penetrating activity of the cell penetrating peptide.

In some embodiments, the IL-17RB inhibitory peptide of the present invention comprising the motif of SEQ ID NO: 10, or the motif of SEQ ID NO: 11, 12 or 13, or the particular amino acid sequence selected from the group consisting of SEQ ID Nos: 14-20 or its variant having substantially identical amino acid sequence thereto, optionally fused to a cell-penetrating peptide, has a length of at least 14 amino acids and less than 80 amino acids, particularly less than 70 amino acids, more particularly less than 60 amino acids, even more particular less than 50 amino acids.

The IL-17RB inhibitory peptide of the present invention may be produced by chemical synthesis using techniques well known in the chemistry of proteins such as solid phase synthesis or synthesis in homogenous solution.

As an alternative, the IL-17RB inhibitory peptide of the present invention can be prepared using recombinant techniques. In this regard, a recombinant nucleic acid comprising a nucleotide sequence encoding an IL-17RB inhibitory peptide of the present invention are provided.

The term “polynucleotide” or “nucleic acid” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. The term “nucleic acid” typically refers to large polynucleotides. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

The term “complementary” refers to the topological compatibility or matching together of interacting surfaces of two polynucleotides. Thus, the two molecules can be described as complementary, and furthermore the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Thus, the polynucleotide whose sequence 5′-TATAC-3′ is complementary to a polynucleotide whose sequence is 5′-GTATA-3′.”

The term “encoding” refers to the inherent property of specific sequences of nucleotides in a polynucleotide (e.g., a gene, a cDNA, or an mRNA) to serve as templates for synthesis of other polymers and macromolecules in biological processes having either a defined sequence of nucleotides (i.e., rRNA, tRNA and mRNA) or a defined sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “recombinant nucleic acid” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence (expression vector) or maintaining the given nucleotide sequence for replicating it, manipulating it or transferring it between different locations (e.g., between different organisms). Vectors can be introduced into a suitable host cell for the above-mentioned purposes. A “recombinant cell” refers to a host cell that has had introduced into it a recombinant nucleic acid. “Transformation” refers to a genetic change in a cell following incorporation of new DNA (i.e., DNA exogenous to the cell). “Transfection” means the process of a cell being transferred with exogenous DNA. “Transduction” can specifically mean the process whereby exogenous DNA is introduced into a cell via a viral vector. “A transformed cell” mean a cell into which has been introduced, by means of recombinant DNA techniques, a DNA molecule encoding a protein of interest.

Vectors may be of various types, including plasmids, cosmids, fosmids, episomes, artificial chromosomes, phages, viral vectors, etc. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprise, for example and without limitation, a promoter sequence (e.g., the cytomegalovirus (CMV) promoter, simian virus 40 (SV40) early promoter, T7 promoter, and alcohol oxidase gene (AOX1) promoter), a start codon, a replication origin, enhancers, an operator sequence, a secretion signal sequence (e.g., a-mating factor signal) and other control sequence (e.g., Shine-Dalgarno sequences and termination sequences). Preferably, vectors may further contain a marker sequence (e.g., an antibiotic resistant marker sequence) for the subsequent screening procedure. For purpose of protein production, in vectors, the given nucleotide sequence of interest may be connected to another nucleotide sequence other than the above-mentioned regulatory sequence such that a fused polypeptide is produced and beneficial to the subsequent purification procedure. Said fused polypeptide includes a tag for purpose of purification, which can be bound to an end of the polypeptide and preferably is small in size that does not affect the desired activity of the polypeptide. Specifically, the tag is of about 30 amino acid residues or less, particularly about 20 amino acid residues or less, more particularly about 10 amino acid residues or less in length; or has a molecular weight of about 10 kDa or less, particularly about 5 kDa or less, more particularly about 2.5 kDa or less. Examples of such tag include, but is not limited to a six (6) to fourteen (14) His-tag or a one (1) to two (2) Myc-tag. The tag may be connected to an N-terminus or a C-terminus of the polypeptide. In some embodiments, the tag may be cleavable in vitro or in vivo. The in vitro or in vivo cleaving may be processed by a protease.

In some embodiments, the peptide of the present invention can be said to be “isolated” or “purified” if it is substantially free of cellular material or chemical precursors or other chemicals that may be involved in the process of peptide preparation. It is understood that the term “isolated” or “purified” does not necessarily reflect the extent to which the peptide has been “absolutely” isolated or purified e.g. by removing all other substances (e.g., impurities or cellular components). In some cases, for example, an isolated or purified peptide includes a preparation containing the peptide having less than 50%, 40%, 30%, 20% or 10% (by weight) of other proteins (e.g. cellular proteins), having less than 50%, 40%, 30%, 20% or 10% (by volume) of culture medium, or having less than 50%, 40%, 30%, 20% or 10% (by weight) of chemical precursors or other chemicals involved in synthesis procedures. The term “isolated” or “purified” can also apply in the nucleic acid of the present invention.

According to the present invention, an effective amount of the IL-17RB antagonist may be formulated with a physiologically acceptable carrier into a composition of an appropriate form for the purpose of delivery and absorption. The composition of the present invention particularly comprises about 0.1% by weight to about 100% by weight of the active ingredient, wherein the percentage by weight is calculated based on the weight of the whole composition. In some embodiments, the composition of the present invention can be a pharmaceutical composition or medicament for treatment.

As used herein, “physiologically acceptable” means that the carrier is compatible with the active ingredient in the composition, and preferably can stabilize said active ingredient and is safe to the receiving individual. Said carrier may be a diluent, vehicle, excipient, or matrix to the active ingredient. Some examples of appropriate excipients include lactose, sucrose, dextrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents. The composition of the present invention can provide the effect of rapid, continued, or delayed release of the active ingredient after administration to the patient.

According to the present invention, the form of the composition may be tablets, pills, powder, lozenges, packets, troches, elixers, suspensions, lotions, solutions, syrups, soft and hard gelatin capsules, suppositories, sterilized injection fluid, and packaged powder.

The composition of the present invention may be delivered via any physiologically acceptable route, such as oral, parenteral (such as intramuscular, intravenous, subcutaneous, and intraperitoneal), transdermal, suppository, and intranasal methods. Regarding parenteral administration, it is preferably used in the form of a sterile water solution, which may comprise other substances, such as salts or glucose sufficient to make the solution isotonic to blood. The water solution may be appropriately buffered (e.g. with a pH value of 3 to 9) as needed. Preparation of an appropriate parenteral composition under sterile conditions may be accomplished with standard pharmacological techniques well known to persons skilled in the art.

To practice the method disclosed herein, an effective amount of a composition such as a pharmaceutical composition described herein, comprising an IL-17RB antagonist can be administered to a subject (e.g., a human) in need of the treatment via a suitable route. The term “effective amount” used herein refers to the amount of an active ingredient to confer a desired biological effect in a treated subject or cell. The effective amount may change depending on various reasons, such as administration route and frequency, body weight and species of the individual receiving said pharmaceutical, and purpose of administration. Persons skilled in the art may determine the dosage in each case based on the disclosure herein, established methods, and their own experience.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder, such as cancer. A subject suspected of having any of such target disease/disorder might show one or more symptoms of the disease/disorder. A subject at risk for the disease/disorder can be a subject having one or more of the risk factors for that disease/disorder.

In some embodiments, abnormal IL-17RB activation is associated with a proliferation disorder e.g. a cancer and a metastasis thereof.

In certain embodiments, the cancer is selected from the group consisting of lung cancer, pancreatic cancer, breast cancer, colorectal cancer, liver cancer, kidney cancer, head and neck cancer, esophageal cancer, gastric cancer, biliary tract cancer, gallbladder and bile duct cancer, mammary cancer, ovarian cancer, cervical cancer, uterine body cancer, bladder cancer, prostate cancer, testicular tumor, osteogenic and soft-tissue sarcomas, leukemia, malignant lymphoma, multiple myeloma, skin cancer, brain tumor and pleural malignant mesothelioma.

In one particular example, the cancer is breast cancer. In another particular example, the cancer is pancreatic cancer.

The present invention is also based on identification of phosphorylated IL-17RB as a marker of poor prognosis of cancer. As demonstrated in the examples below, pancreatic cancer patients with higher expression level of phosphorylated IL-17RB protein are observed to have lower survival rate and higher metastases than those with lower expression level of phosphorylated IL-17RB protein.

Therefore, the present invention provides a method for predicting prognosis of cancer e.g. a pancreatic cancer based on the level of phosphorylated IL-17RB protein.

As used herein, the term “prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. It would be understandable that a positive prognosis typically refers to a beneficial clinical outcome or outlook, such as long-term survival without recurrence of the subject's cancerous conditions, whereas a negative (poor) prognosis typically refers to a negative clinical outcome or outlook, such as cancer recurrence or progression. In certain embodiments, the negative prognosis is selected from the group consisting of a reduced survival rate, an increased tumor size or number, an increased risk of metastasis, an increased risk of relapse, and any combination thereof.

In some embodiments, the method of the present invention comprises measuring an expression level of phosphorylated IL-17RB protein (particularly phosphorylation at position Y447) in a sample obtained from a cancer patient and determining the prognosis of cancer in the patient based on the expression level of phosphorylated IL-17RB protein in the sample, wherein an elevated level of phosphorylated IL-17RB protein in the sample indicates poor prognosis.

As used herein, an elevated level means a level that is increased compared with the level in a subject free from the cancer or a reference or control level. For example, an elevated level can be higher than a reference or control level by more than 10%, 20%, 30%, 40%, 50%, 60%, 70%, 80%, 90% or 100%. A reference or control level can refer to the level measured in normal individuals or sample types such as tissues or cells that are not diseased.

As used herein, “low expression” and “high expression” for a biomarker as used herein are relative terms that refer to the level of the biomarker found in a sample. In some embodiments, low and high expression can then be assigned to each sample based on whether the expression of such biomarker in a sample is above (high) or below (low) the average or median expression level in a population of cancer patients. Typically, such population of cancer patients are chosen to be matched to the candidate individual in, for example, age and/or ethnic background. Preferably, such population of cancer patients and the candidate individual are of the same species. In some embodiments, low expression can be defined as the percentage of positive staining of cell populations in a tissue section less than 30%, 20%, 10% or 5% and high expression is defined as the percentage of positive staining of cell populations in a tissue section more than 30%, 20%, 10% or 5%.

To perform the methods described herein, a biological sample can be obtained from a subject in need and a marker in the biological sample can be detected or measured via any methods known in the art, such as immunoassays. A higher level of the marker as detected in a biological sample from the candidate subject can indicate that the candidate subject has a negative prognosis of cancer. In some examples, the level of the marker(s) in a control sample is undetectable in a control sample (i.e. the reference value being 0), and the presence of the marker as detected in a biological sample from a subject can indicate that the subject has a negative prognosis of cancer. In some examples, the level of the marker(s) can be measured at different time points in order to monitor the progression of the cancer. For example, two biological samples are obtained from a candidate subject at two different time points. If a trend of increase in the level of the marker(s) is observed over time, for example, the level of the marker(s) in a later obtained sample is higher than that in an earlier obtained sample, the subject is deemed to have cancer progression.

In some embodiments, the method of the present invention comprises
(a) measuring a level of phosphorylated IL-17RB protein in a first biological sample obtained from the patient at a first time-point;
(b) measuring a level of phosphorylated IL-17RB protein in a second biological sample obtained from the patient at a second time-point; and
(c) determining cancer progression in the patient based on the levels in the first and second biological samples wherein an elevated level of phosphorylated IL-17RB protein in the second biological sample as compared to that in the first biological sample is indicative of cancer progression.

In some embodiments, the presence and/or amount of a biomarker can be determined by an immunoassay. Examples of the immunoassays include, but are not limited to, Western blot, enzyme-linked immunosorbent assay (ELISA), radioimmunoassay (RIA), radioimmunoprecipitation assay (RIPA), immunofluorescence assay (IFA). ELFA (enzyme-linked fluorescent immunoassay), electrochemiluminescence (ECL), and Capillary gel electrophoresis (CGE). In some examples, the presence and/or level of a biomarker can be determined using an agent specifically recognizes said biomarker, such as an antibody that specifically binds to the biomarker.

In other embodiments, the presence and/or amount of a biomarker can be determined by measuring mRNA levels of the one or more genes. Assays based on the use of primers or probes that specifically recognize the nucleotide sequences of the genes as described may be used for the measurement, which include but are not limited to reverse transferase-polymerase chain reaction (RT-PCR) and in situ hybridization (ISH), the procedures of which are well known in the art. Primers or probes can readily be designed and synthesized by one of skill in the art based on the nucleic acid region of interest. It will be appreciated that suitable primers or probes to be used in the invention can be designed using any suitable method in view of the nucleotide sequences of the genes of interest as disclosed in the art.

Antibodies as used herein may be polyclonal or monoclonal. Polyclonal antibodies directed against a particular protein are prepared by injection of a suitable laboratory animal with an effective amount of the peptide or antigenic component, collecting serum from the animal, and isolating specific sera by any of the known immunoadsorbent techniques. Animals which can readily be used for producing polyclonal antibodies as used in the invention include chickens, mice, rabbits, rats, goats, horses and the like. In one example, an anti-phosphorylated IL-17RB at Y447 is used to perform the method of the present invention.

When an individual, such as a human patient, is diagnosed as having a negative prognosis, the individual may undergo further testing (e.g., routine physical testing, including surgical biopsy or imaging methods, such as X-ray imaging, magnetic resonance imaging (MRI), or ultrasound) to confirm the occurrence of the disease and/or to determine the stage and progression of cancer.

In some embodiments, the methods described herein can further comprise treating the cancer patient to at least relieve symptoms associated with the disease. The treatment can be any conventional anti-cancer therapy, including radiation therapy, chemotherapy, and surgery.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

The members of the Interleukin-17 (IL-17) cytokine family and their receptors (IL-17R) were identified decades ago. Unlike IL-17 receptor A (IL-URA), which hetero-dimerizes with IL-17RB, -C, and -D and mediates pro-inflammatory gene expression, IL-17B/IL-17RB plays a distinct role in promoting tumor growth and metastasis. However, the molecular basis of how the oncogenic signaling of IL-17RB is initiated and transmitted remains elusive. Here, we report that IL-17RB forms a homo-dimer and recruits MLK4, a dual kinase, to phosphorylate it at tyrosine 447 upon treatment with IL-17B ligand. The tyrosine phosphorylated IL-17RB, which is present in higher amounts in the cells of pancreatic cancer cases with poor prognosis, recruits the ubiquitin ligase TRIM56. TRIM56 adds K63-linked ubiquitin chains to lysine 470 of IL-17RB, which further assembles Act1 and other factors to propagate downstream oncogenic signaling. Consistently, both Y447F and K470R IL-17RB mutants lose this oncogenic activity. Treatment with a specific peptide consisting of amino acids 403-416 of IL-17RB blocks MLK4 binding, Y447phosphorylation, and K470 ubiquitination, thereby inhibiting tumorigenesis and metastasis and prolonging the lifespan of mice bearing pancreatic tumors. These results not only establish a clear pathway of how proximal signaling of IL-17RB occurs, but also provides insight into how this pathway is clinically significant in many pancreatic and breast cancers.

### 1. Material and Methods

### 1.1 Cell Culture, Transfection and Reagent Treatment

Human embryonic kidney cell line HEK293T, human pancreatic cancer cell lines AsPC-1, BxPC-3, CFPAC-1, human breast cancer cell lines MDA-MB-361 and MDA-MB-468 were purchased from ATCC and cultured in a humidified 37° C. incubator supplemented with 5% CO₂. These cell lines used in this paper are not listed in the database of misidentified cell lines in NCBI Biosample and were not further authenticated after purchase. HEK293T, AsPC-1, MDA-MB-361 and MDA-MB-468 cells were maintained in Dulbecco's modified Eagle's medium (DMEM), BxPC3 cells were cultured in RPMI-1640 medium, and CFPAC1 cells were cultured in Iscove's Modified Dulbecco's Medium (IMDM). All the media were supplemented with 10% fetal bovine serum, penicillin and streptomycin (100 IU/ml and 100 μg/ml, respectively), and 1× non-essential amino acids. All the media and supplements were purchased from Gibco (Thermo Fisher Scientific). These cell lines were regularly examined for mycoplasma contamination by DAPI staining and e-Myco Mycoplasma PCR Detection Kit (Bulldog Bio.). TransIT-LT1 Transfection Reagent (MIR 2300, Mirus Bio) was used for the transient transfection of plasmids into cells according to the manufacturer's instructions. Pan-mixed lineage kinase (Pan-MLK) inhibitor CEP-1347 was purchased from Tocris Bioscience. Peptides of TAT₄₈₋₅₇ (control) and TAT-IL-17RB₄₀₃₋₄₁₆ (loop peptide) were purchased from TOOLs with purity>95%. To assess the effect of IL-17B on IL-17RB signaling, cells were cultured in serum-free medium for 2 hrs before adding 50 ng/ml rIL-17B to eliminate endogenous IL-17B interference.

### 1.2 Co-Immunoprecipitation (Co-IP) Assay and Immunoblotting Analysis

Whole-cell lysates were prepared as previously described (6, 7). Briefly, 500 μg of crude whole-cell extract was incubated with 5 μg anti-IL-17RB (FGRB, in-house-generated mouse polyclonal) or control normal mouse IgG (mIgG, 015-000-003, Jackson ImmunoResearch) antibodies at 4° C. overnight. Then, 50 μl pre-washed protein A/G agarose beads (sc-2003, Santa Cruz Biotechnology) were added to the mixture and incubated at 4° C. for 2 hrs with gentle agitation. For reciprocal co-IP, the whole-cell extract was incubated with anti-Flag M2 agarose (A2220, Sigma-Aldrich) or anti-HA (HA-7) agarose (A2095, Sigma-Aldrich) at 4° C. for 2 hrs with gentle agitation. After extensive washing with diluted lysis buffer (0.01% Triton X-100), IL-17RB and its associated proteins were analyzed by immunoblotting analysis. Bradford assay (Bio-Rad) was used for determining protein concentration.

Immunoblotting analysis was performed after proteins were separated by gradient SDS-PAGE (HR gradient gel solution, TFUGG420, TOOLS) and transferred to PVDF membrane, with overnight incubation with diluted primary antibody followed by diluted horseradish peroxidase-conjugated anti-rabbit, anti-mouse or anti-goat antibody (Jackson ImmunoResearch) as described (48-50). Mouse monoclonal antibody against IL-17RB (A68) and rabbit anti-P-Y447 polyclonal antibody were generated in-house. Other primary antibodies used include anti-phosphoserine (P5747), anti-Flag (clone M2) and anti-HA (clone HA-7) from Sigma-Aldrich, anti-phosphothreonine (clone RM102), anti-AAK1 (GTX₅₉₆₆₃), anti-HIPK1 (clone C3) and anti-GAPDH antibody (GTX₆₂₇₄₀₈) from GeneTex, anti-phosphotyrosine (clone PY20) from Merck, anti-p-ERK1/2 (4370) and anti-ERK1/2 (4695) from Cell Signaling, anti-MLK4 (ab93798) from Abcam, anti-6-His tag (A109-114) from Bethyl Labs. Immunoblot Signals were developed using the Clarity™ Western ECL Blotting Substrates and ChemiDoc™ Imaging Systems (Bio-Rad). Optical density was determined using the National Institutes of Health ImageJ program.

### 1.3 Preparation of Plasmids, Retroviruses and Lentiviruses

Retro-neo control and IL-17RB were cloned into the retroviral vector as previously described (6, 7). The IL-17RB mutants including Y338F, Y350F, Y443F, Y447F, Y457F and Y466F were generated using the QuickChange XL site-directed mutagenesis kit (200516, Agilent) according to manufacturer's instructions. The WT and mutant pQCXIP-IL-17RB plasmids were individually co-transfected with pMD.G (Env-encoding vector) into Gp2-293 cells to generate retroviruses carrying IL-17RB.

Flag-MLK4 and Flag-IL-17RB cloned in pCMV6-Entry were purchased from OriGene. Full-length IL-17RB was subcloned into EcoRI/EcoRV sites of pcDNA3.1 plasmid for expressing C-terminally HA-tagged IL-17RB. IL-17RA-His was constructed by insertion of cDNA of IL-17RA at HimDIII site and BamHI site of pCNA3.1+/myc-His A vector. TRIM56-HA was constructed by insertion of cDNA of TRIM56 at HimDIII site and BamHI site of pCNA3.1+/C-HA vector. All the pcDNA3-His-Ubiquitin clones were kindly provided by Dr. Ruey-Hwa Chen at Institute of Biological Chemistry, Academia Sinica, Taiwan.

Flag-tagged IL-17RB mutants (FNmut and Y447F), HA-tagged IL-17RB mutants (ΔH346˜F354, ΔI373˜T384, ΔV403˜S416, ΔF423˜F430, S423˜F430, ΔN458˜V462, ΔK470˜Q484 and ΔQ484˜S502), MLK4 mutants (Δ100-108, E314K and Y330H), and TRIM56 mutants (Δ31-50) were also generated using the QuickChange XL site-directed mutagenesis kit (200516, Agilent).

The lentiviral shRNA expression vectors of pLK0.1-shLacZ, shMLK4 (mixture of TRCN3212 and TRCN3213), shAAK1 (mixture of TRCN1945 and TRCN1945), shHIPK1 (mixture of TRCN7163 and TRCN7165), TRIM56 (TRCN73094, 73096), ACTT (TRCN162747, 163987), and TRAF6 (TRCN7349, 7350, 7351, 7352), packaging plasmid pCMVΔR8.91, and pMD.G were obtained from the National RNAi Core Facility of Academia Sinica (Taipei, Taiwan). For lentivirus production, 293T cells were transfected with 5 μg pLKP.1-puro lentiviral vectors expressing different shRNAs along with 0.5 μg of envelope plasmid pMD.G and 5 μg of packaging plasmid pCMVΔR8.91 as described (7). Viruses were collected 48 hrs after transfection. To establish cells depleted with IL-17RB or MLK4, different cell lines including AsPC1, BxPC3, CFPAC1, MDA-MB-361 and MDA-MB-468 were infected with lentiviruses containing the corresponding shRNA for 24 hrs and then selected with appropriate antibiotics.

### 1.4 Genome-Editing of IL-17RB, MLK4 and TRIM56 Gene in the Pancreatic Cancer Cell

To introduce DNA double-strand break repair-dependent gene deletion or mutation in IL-17RB, MLK4 and TRIM56 in BxPC3 cells, we used RNA-guided endonucleases (RGENs) system were obtained from the National RNAi Core Facility of Academia Sinica (Taipei, Taiwan) to express the Cas9 endonuclease and the sequences of guide RNA (gRNA) for targeting IL-17RB, MLK4, and TRIM56 were designed and provided. BxPC3 cells were transfected with 10 μg of each plasmid using TransIT-LT1 Transfection Reagent (MIR 2300, Mirus Bio) following manufacturer's instruction. After 2 days of transfection, we performed limiting dilution to derive single cell clones and measured the expression of these genes by immunoblotting analysis.

### 1.5 RNA Isolation, Reverse-Transcription, Real-Time RT-PCR Assays

Total RNA from cultured cells and tumor tissue was isolated using Trizol reagent (Thermo Fisher Scientific) and reversely transcribed with Transcriptor First Strand cDNA Synthesis Kit (Roche Life Science) for gene expression analysis according to manufacturer's instructions. Quantitative real-time RT-PCR assay was run on the StepOnePlus system (Applied Biosystems) using KAPA SYBR FAST qPCR Kit (Kapa Biosystems) according to manufacturer's instructions, and data was analyzed by StepOne Software v2.2.2. β-actin mRNA was used as an internal control. Expression levels were calculated according to the relative ACt method as described (7).

### 1.6 Soft Agar Colony Formation Assay and Invasion Assay

Soft agar colony formation assay was performed as described (7). Briefly, 250010000 cells were seeded in a layer of 0.35% agar/complete growth medium over a layer of 0.5% agar/complete growth medium in a 12-well plate. Cell medium containing the indicated concentration of rIL-17B (R&D), DMSO (Sigma-Aldrich), or CEP-1347 (Tocris Bioscience) was replenished every three days. On day 14 or day 21 after seeding, cells were fixed and stained with pure ethanol containing 0.05% crystal violet (Sigma-Aldrich). Crystal violet-stained colonies greater than 50 μm in size were counted and analyzed objectively by light microscopy.

For invasion assay, about 10⁴ cells were seeded in the top chamber with Matrigel-coated membrane (24-well BD Falcon HTS Fluoro Block insert; pore size, 8 μm; BD Biosciences) in serum-free media containing rIL-17B, DMSO, or CEP-1347. Medium supplemented with 10% serum was used as a chemoattractant in the lower chamber. After 24- or 48 hrs incubation, the invading cells were fixed with methanol, stained with 4,6-diamidino-2-phenylindole (DAPI) and counted by fluorescence microscopy.

### 1.7 Mass Spectrometry

CFPAC1 cells were treated with 50 ng/ml rIL-17B or bovine serum albumin for 30 mins in the serum-free condition after serum starvation for 2 hrs. Then, the culture media were removed and the cells were washed with PBS. Crosslinker DSP (Thermo Fisher Scientific) dissolved in PBS was used to treat the cells for 30 mins and then stopped the reaction by adding Tris buffer (50 mM, pH 8.0). Whole cell lysate was collected and incubated with anti-IL-17RB antibody for co-immunoprecipitation. The IL-17RB-interacting proteins were separated by SDS-PAGE and the protein bands from SDS-PAGE were subjected to in-gel trypsin/chymotrypsin digestion following standard procedure, and then analyzed by mass spectrometry. The procedures and data analysis were performed as described (48). Briefly, the enzyme-digested protein samples were injected onto a self-packed precolumn (150 μm I.D.×20 mm, 5 μm, 200 Å). Chromatographic separation was performed on self-packed reversed-phase C18 nano-column (75 μm I.D.×300 mm, 5 μm, 100 Å) using 0.1% formic acid in water (mobile phase A) and 0.1% formic acid in 80% acetonitrile (mobile phase B). A linear gradient was applied from 5-45% mobile phase B for 40 min at a flow rate of 300 nL/min. Electrospray voltage was applied at 2 kV, and capillary temperature was set at 200° C. A scan cycle was initiated with a full-scan survey MS spectrum (m/z 300-2,000) performed on a FT-ICR mass spectrometer with a resolution of 100,000 at 400 Da. The ten most abundant ions detected in this scan were subjected to a MS/MS experiment performed in the LTQ mass spectrometer. Ion accumulation (Auto Gain Control target number) and maximum ion accumulation time for the full scan and MS/MS were set at 1×10⁶ ions, 1,000 ms and 5×10⁴ ions, 200 ms, respectively. Ions were fragmented by use of collision-induced dissociation (CID) with the normalized collision energy set to 35%, activation Q set to 0.3 and an activation time of 30 ms.

### 1.8 Analysis of IL-17 Receptor Oligomerization by Immunoprecipitation

BxPC3 IL-17RB-KO cells were transiently co-transfected with IL-17RB-HA. IL-17RB-Flag and IL-17RA-His plasmids using TransIT-LT1 Transfection Reagent (MIR 2300, Mirus Bio) according to manufacturer's instructions. The cells were then treated with various concentrations of rIL-17B or rIL-17E for the indicated times and lysed for co-immunoprecipitation assay using different antibodies as described above.

### 1.9 Duolink In Situ Interaction Assay

DuoLink proximity ligation assay (PLA) Kit (DuoLink, DU092101, Sigma-Aldrich) was used to detect protein—protein interaction using fluorescence microscopy as in the manufacturer's protocol. Briefly, the pancreatic cancer cells were treated with rIL-17B at the indicated concentration for 30 mins in eight-chamber microscope slides after serum starvation for 2 hrs, fixed with 4% paraformaldehyde for 15 mins at room temperature, permeabilized with 0.2% Triton X-100 and blocked with DuoLink blocking buffer for 30 mins at 37° C. The cells were then incubated with primary antibodies diluted in DuoLink antibody diluents for 1 hr. These different primary antibodies sets were used in each distinct experiment; antibodies against IL-17RB (FGRB, home-made) and MLK4 (Abram, ab93798, rabbit) were used in FIG. 3F, and antibodies against Flag tag (Sigma-Aldrich, F7425, mouse) and HA tag (Sigma-Aldrich, HA-7, rabbit) were used in FIG. 4C and FIG. 13D, and antibodies against IL-17RB (D9) native form were used in FIG. 4D. After that, the cells were washed and further incubated for 1 hrs at 37° C. with species-specific secondary antibody carrying PLA probes for hybridization, which was facilitated in close proximity (<16 nm) in the presence of two complementary oligonucleotides. After annealing and rolling-circle amplification, a detection solution consisting of fluorescently labeled oligonucleotides was added, and the slides were mounted with Fluoroshield with DAPI (GeneTex). The signal was detected as distinct fluorescent dots in the Texas red channel. Microscopy images were acquired by confocal spectral microscopy (Leica SP2/SP8X) and analyzed by LAS AF software (Leica Biosystems). Negative controls consisted of samples treated as described above but with secondary antibodies alone.

### 1.10 Immunohistochemistry (IHC)

Formalin-fixed paraffin-embedded primary tumor tissue sections were used for IHC. Heat-induced antigen retrieval was performed using 0.1M citrate buffer, pH 6.0 and autoclaved for 20 mins. Endogenous peroxidase was eliminated with 3% H₂O₂. Slides were stained with an in-house-generated anti-IL-17RB antibody (A81, 1:2000) and anti-P-Y447 (1:500), respectively in PBS/10% FBS overnight at 4° C. After washing, slides were incubated with anti-rabbit/mouse HRP polymer before visualization with liquid diaminobenzidine tetrahydrochloride plus substrate DAB chromogen from Dako REAL EnVision (Carpinteria, CA). All slides were counterstained with hematoxylin.

### 1.11 Retrospective Cohort Study for Validation of the Prognostic Marker

For the independent validation of the P-Y447 IL17RB as a prognostic marker, an independent cohort of 87 patients with pancreatic cancer who received surgical resection in National Taiwan University Hospital (NTUH) from 2007 to 2015 was used. The pancreatic cancer specimens were collected from patients who underwent pancreatic duodenectomy and had pathologically confirmed PDAC. Clinical characteristics of patients in the validation cohort were listed in Table 4. The data of the clinical relevance is reported according to REMARK guidelines in this paper.

### 1.12 Tumor Infiltrate Analysis by Flow Cytometry

Tumors were excised, weighed and measured. Approximately half of each dissected tumor was fixed in formalin for histopathologic analysis. The remaining tumor sections were placed in PBS with 1% (v/v) FBS and mechanically minced. Minced tumors were further dissociated in the solution containing 0.4 mg/mL collagenase P and 0.1 ng/mL DNase I dissolved in Hanks' Balanced Salt Solution (HBSS) at 37° C. for 30 mins. These cell pellets were resuspended in PBS and stained for Flow cytometry analysis using the following antibodies provided by BD Bioscience (San Jose, CA, USA) to identify immune and tumor cell subsets based on their cell surface markers.

The following monoclonal antibodies and reagents were used: BUV395 anti-CD45 (clone 30-F11), PerCP-Cy5.5 anti-CD4 (clone GK1.5), APC-H7 anti-CD8a (clone 53-6.7), Alexa Fluor647 anti-FoxP3 (clone MF23), PerCP-Cy5.5 anti-CD11b (clone M1/70), APC-H7 anti-Ly6G (Gr1, clone 1A8), APC-H7 anti-CD11c (clone HL3), PerCP-Cy5.5 anti-NK1.1 (clone PK136), Alexa Fluor647 anti-F4/80 (TA45-2342) and Alexa Fluor647 anti-CD206 (clone MR53D). Flow cytometric analysis was run by a BD Biosciences LSRII (Franklin Lakes, NJ, USA) flow cytometer, and data were analyzed by FlowJo software (Ashland, OR, USA).

### 1.13 Human Studies

All pancreatic cancer patient data and tissue specimens were from the National Taiwan University Hospital (NTUH), Taipei, Taiwan (Table 3 and Table 4), approved by the Institutional Review Board of the NTUH (201701015RINA).

### 1.14 Animal Studies

All animal experiments described herein were approved by the Institutional Animal Care and Utilization Committee of Academia Sinica (IACUC #16-03-945) and China Medical University (IACUC #2017-015), Taiwan. Male mice ˜8-12 weeks old were randomized to groups in unbiased fashion. The investigators were blinded to group allocation during experiments and outcome assessment. Based on pilot experiments, sample sizes were estimated to provide sufficient numbers of mice in each group for statistical analysis.

For the spontaneous pancreatic cancer model; Kras+/LSLG12D mice (B6; 129-Kras2) bearing the Cre-dependent conditional knock-in mutation KrasG12D were obtained from Mouse Models of Human Cancer Consortium (48). Kras+/LSLG12D mice were bred with Elas-CreER mice obtained from the Level Transgenic Center (Taipei, Taiwan) (33-35, 49), to generate Elas-CreER^{T};Kras+/LSLG12D mice. To induce the expression of Kras+/LSLG12D in acinar cells, five-week-old Elas-CreER^{T};Kras+/LSLG12D male mice were intraperitoneally injected with free base tamoxifen (20 mg/mL in corn oil; Sigma-Aldrich) three times per week (three injections of 2 mg each) for one week. Those mice were then intraperitoneally injected with cerulein (50 μg/mL in PBS; BACHEM) six times per week (six injections of 5 μg each) for three weeks to induce chronic inflammation and tumors. The animals were intraperitoneally injected with PBS, TAT peptide (control) or TAT-IL-17RB403-416 peptide and monitored for lifespan and tumor size.

For the orthotopic pancreatic cancer model, human pancreatic tumor cells expressing GFP/Luc were injected into six-week-old NOD-SCID female mice. These mice were first anaesthetized using continuous isoflurane, and their abdomen was sterilized. We then performed a laparotomy (5-10 mm) over the left upper quadrant of the abdomen to expose the peritoneal cavity. The pancreas was exteriorized onto a sterile field, and sterile PBS or 5×10⁵ of pancreatic tumor cells suspended in 25 μl of sterile PBS were injected into the tail of the pancreas via a 30-gauge needle (Covidien). Successful injection was confirmed by the formation of a liquid bleb at the site of injection with minimal fluid leakage. The pancreas was then gently placed back into the peritoneal cavity. The peritoneum was then closed with a 5-0 PDS II violet suture (Ethicon), and the skin was closed using the AutoClip system (Braintree Scientific). In vivo bioluminescence signals were assessed and analyzed with the IVIS Spectrum In Vivo Imaging System (PerkinElmer).

### 1.15 Study Design

The objective of our study was to elucidate the proximal transduction mechanisms by which IL-17-receptor B binds to its ligand, IL-17B, for oncogenic signaling pathway. The first clue was obtained by searching for receptor phosphorylation by immunoblotting analysis with antibodies against modified residues. A specific antibody against Y447 phosphorylated IL-17RB was made and used to search for the responsible kinase by proteomic analysis. MLK4 was identified as the kinase to phosphorylate IL-17RB and validated its importance by standard oncogenic activity assays. Next, the MLK4-binding site of IL-17RB was mapped by deletion analysis and validated by the loop peptide for blocking the interaction and inhibiting tumor malignancy in mice models. Further key step of the downstream signal was similarly explored and ubiquitin ligase TRIM56 was identified for adding K63-linked ubiquitin chains to lysine 470 of IL-17RB after P-Y447. All the experiments were repeated at least twice.

### 1.16 Statistical Analysis

We chose our sample sizes based on those commonly used in this field without predetermination by statistical methods. Except for the clinical correlation and quantification for specific immunoblots, all data were presented as means±SD, and two-tailed Student's t-test was used to compare control and treatment groups. Asterisk (*) and (**) indicate statistical significance with p-value<0.05 and <0.01, respectively. The following analyses were performed using GraphPad Prism 6 and SPSS statistics software. The Kaplan-Meier estimation method was used for overall survival analysis of the tumor-bearing mice, and a log-rank test was used to compare differences.

### 2. Results

### 2.1 Phosphorylation of Y447 Is Critical for IL-17B/IL-17RB Oncogenic Signal Transduction

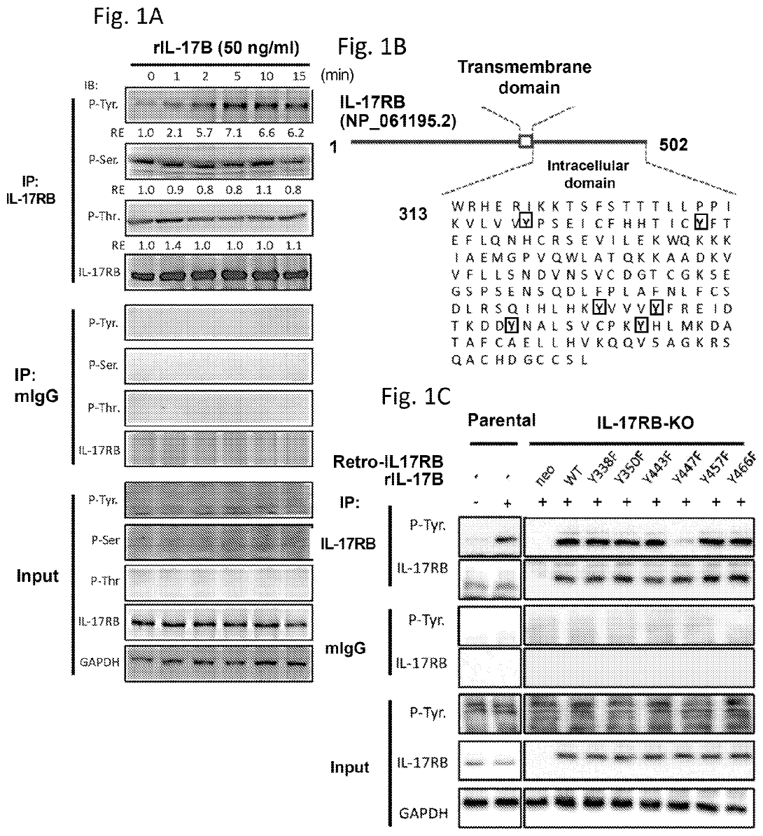

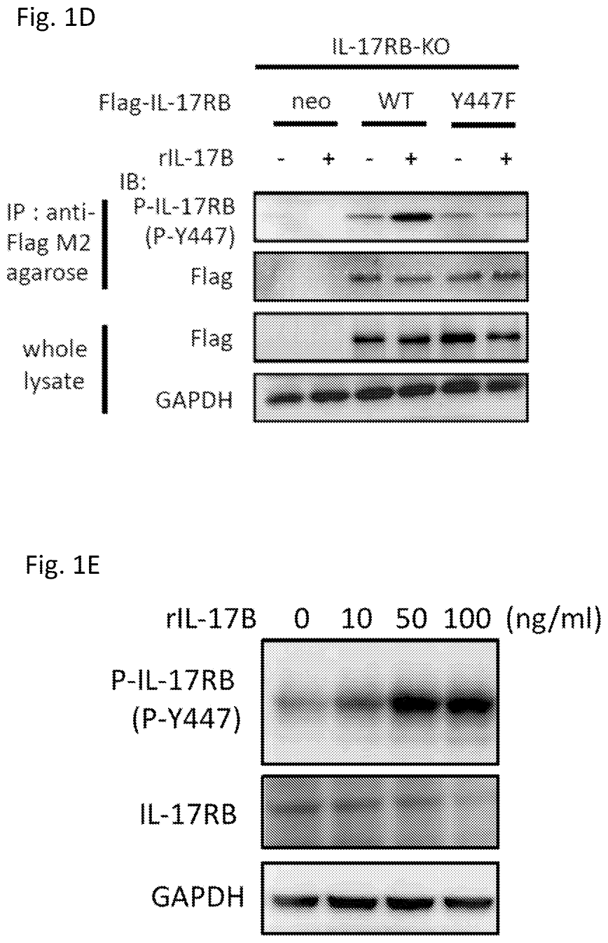

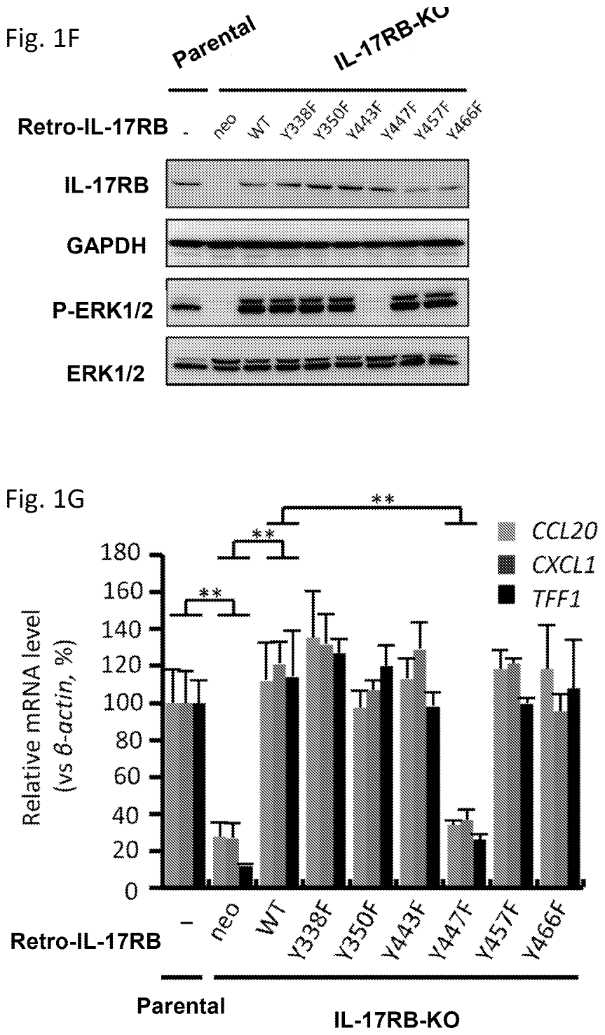

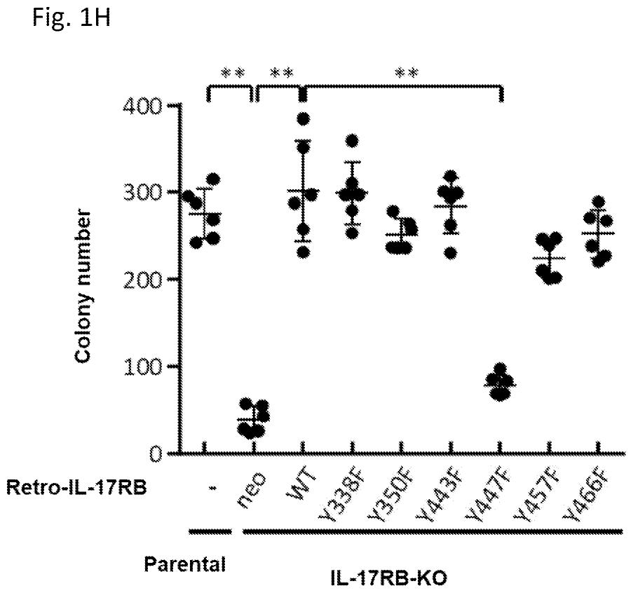

To explore the molecular basis of how IL-17B/IL-17RB signaling initiates, we employed a two-pronged approach by identifying post-transcriptional modification (PTM) residues of IL-17RB and searching for its interacting enzymes through proteomic analysis. Since the earliest response for the majority of cell surface receptors is phosphorylation upon ligand binding, we first tested whether IL-17RB is phosphorylated at tyrosine, serine or threonine residues upon IL-17B stimulation. As shown in FIG. 1A, increasing phosphorylation on tyrosine, but not on serine or threonine, of IL-17RB was observed within five minutes of IL-17B addition (FIG. 1A), suggesting tyrosine phosphorylation of IL-17RB is important for signaling. To determine which tyrosine residue was phosphorylated, we individually changed each of the six tyrosine residues in the intracellular domain (ICD) to phenylalanine (F) (FIG. 1B). We expressed the wild-type and six mutants in IL-17RB-knockout pancreatic cancer cells, in which both cytokine gene expression and the associated aggressive phenotype were lower than the parental cells (FIGS. 8A-8D), and found that the Y447F mutant lost tyrosine phosphorylation induced by IL-17B (FIG. 1C). A cross-species analysis revealed that Y447 is highly conserved in mammals (FIG. 9A). Therefore, we generated a rabbit anti-IL-17RB (P-Y447) antibody, recognizing Y447 phosphorylation (FIG. 1D), and demonstrated that Y447 phosphorylation of IL-17RB was upregulated by IL-17B in a dose-dependent manner (FIG. 1E). Consequently, we evaluated these cells for their downstream signaling, including ERK1/2 phosphorylation, cytokine gene expression, and oncogenic behavior, and found that only the cells expressing Y447F exhibited a reduction of IL-17B-induced ERK1/2 phosphorylation (FIG. 1F and FIG. 9B), cytokine gene expression (FIG. 1G and FIG. 9C), and colony formation compared to the WT control (FIG. 1H and FIG. 9D). These results suggested that phosphorylation of Y447 is critical for IL-17B/IL-17RB oncogenic signal transduction.

### 2.2 Abundance of Phospho-IL-17RB (P-Y447) Correlates With Pancreatic Tumor Malignancy and Worse Prognosis

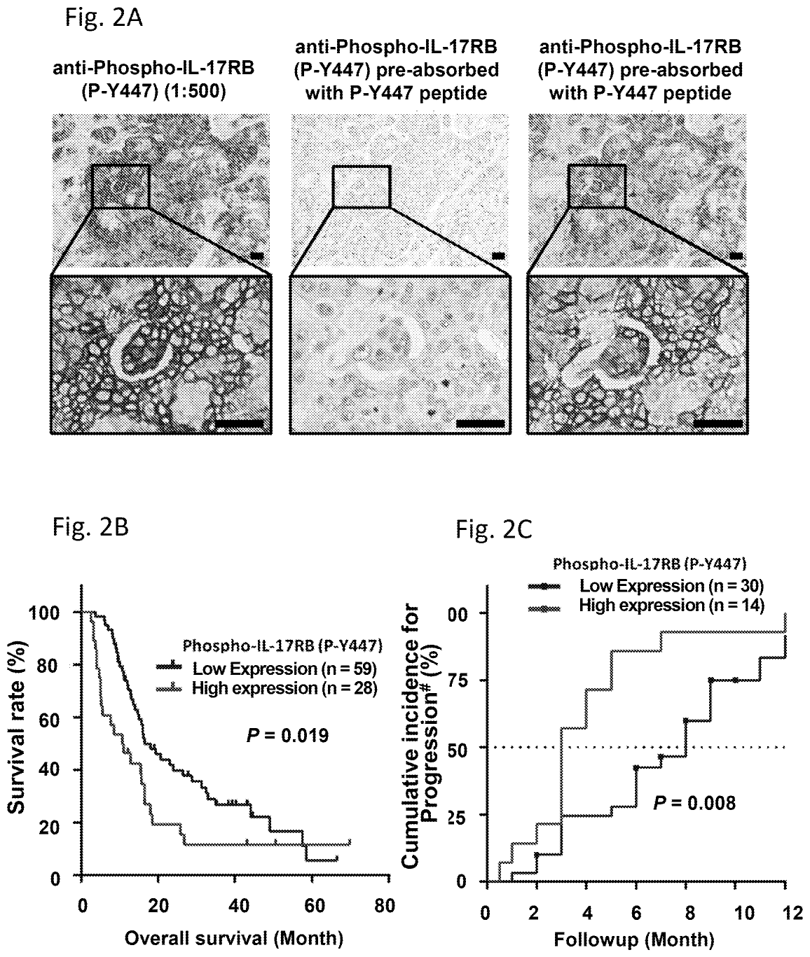

To determine the clinical significance of IL-17RB Y447 phosphorylation, immunohistochemistry (IHC) with the phospho-IL-17RB (P-Y447) antibody (FIG. 2A) was performed. P-Y447 was detected on cell membrane (FIGS. 10A-10B) and associated with the expression of IL-17RB assessed by a monoclonal antibody against IL-17RB [A81] (Table 1 and FIG. 10A).

TABLE 1
Correlation between IL-17RB expression and IL-17RB phosphorylation
(P-Y447) in 87 patients with pancreatic cancer.

| IHC | Total | Phospho-IL-17RB (P-Y447) | | | | |
|---|---|---|---|---|---|---|
| staining | N | Low, n (%) | | High, n (%) | | P value |
| | 87 | 59 | (68) | 28 | (32) | |
| IL-17RB (A81) | | | | | | |
| Low | 51 | 51 | (100) | 0 | (0) | P < 0.001 |
| High | 36 | 8 | (22) | 28 | (78) | |

Importantly, high amounts of P-Y447 IL-17RB correlated with worse prognosis in overall survival of patients with pancreatic cancer (FIG. 2B), and thus P-Y447 may be an independent prognostic biomarker (Table 2 and Table 3).

TABLE 2
Overall survival of the 87 patients with pancreatic cancer.
Chi-square test was used.

| | Patient | Hazard Ratio | |
|---|---|---|---|
| Variants | no. (%) | (95% CI) | P value |
| Univariate: | | | |
| Phospho-IL-17RB: | 28 (32) | 1.963 (1.209-3.190) | 0.007 |
| High (vs. Low) | | | |
| Multivariate^{§}: | | | |
| Phospho-IL-17RB: | 28 (32) | 2.314 (1.276-4.196) | 0.006 |
| High (vs. Low) | | | |

^{§}Adjusted with cancer clinical and pathological parameters of age, gender, T value, N value, AJCC stage, Margin involvement, lymphovascular invasion, and perineuronal invasion (Table 4).

TABLE 3
Univariate and multivariate Cox regression analysis of the influence
of IL-17RB phosphorylation on the overall survival.

| | Patient | Hazard Ratio | |
|---|---|---|---|
| Variants | no. (%) | (95% CI) | P value |
| Univariate: | | | |
| P-Y447: High (vs. Low) | 28 (32) | 1.963 (1.209-3.190) | 0.007 |
| Multivariate: | | | |
| P-Y447: High (vs. Low) | 28 (32) | 2.314 (1.276-4.196) | 0.006 |
| Age: >=60 yr (vs. <60 yr) | 55 (63) | 1.145 (0.672-1.950) | 0.618 |
| Gender: Male (vs. Female) | 52 (60) | 1.178 (0.688-2.018) | 0.551 |
| T value: III (vs II) | 76 (87) | 1.263 (0.473-3.372) | 0.642 |
| N value: Positive (vs Negative) | 50 (57) | 1.005 (0.548-1.841) | 0.988 |
| AJCC stage: IIA & | 82 (94) | 1.392 (0.306-6.336) | 0.668 |
| IIB (vs IA & IB) | | | |
| Grade | | | 0.015 |
| II (vs. I) | 59 (67) | 0.768 (0.370-1.597) | |
| III (vs. I) | 12 (14) | 2.190 (0.905-5.299) | |
| Margins involvement: | 17 (20) | 1.990 (1.005-3.940) | 0.048 |
| involved (vs Free) | | | |
| Lymphovascular invasion: | 60 (69) | 0.706 (0.388-1.287) | 0.256 |
| Positive (vs Negative) | | | |
| Perineural invasion: | 79 (91) | 1.857 (0.702-4.913) | 0.212 |
| Positive (vs Negative) | | | |

Total 87 patients with pancreatic cancer after surgical therapy.

Moreover, tumor specimens with high P-Y447 expression were poorly differentiated (Table 4) and possessed a higher potential to form tumors in mouse xenograft models (Table 5).

TABLE 4
High P-Y447 of IL-17RB expression in pancreatic tumor specimens
correlates with worse clinical progression of the patients.

| | | Phospho-IL-17RB (P-Y447) | | | | |
|---|---|---|---|---|---|---|
| | Total | Low expression | | High expression | | |
| C [symbol] parameters | [symbol] | n (%) | | n (%) | | P value |
| | 87 | 59 | (68) | 28 | (32) | |
| Age | | | | | | |
| <60 | 37 | 20 | (63) | 12 | (37) | 0.418 |
| >=60 | 55 | 39 | (71) | 16 | (29) | |
| Gender | | | | | | |
| Female | 35 | 25 | (77) | 10 | (23) | 0.554 |
| Male | 52 | 34 | (65) | 18 | (35) | |
| T value | | | | | | |
| II | 11 | 6 | (55) | 5 | (45) | 0.313 |
| III | 76 | 53 | (70) | 23 | (30) | |
| N value | | | | | | |
| Negative | 37 | 25 | (68) | 12 | (32) | [symbol] |
| Positive | 50 | 34 | (68) | 16 | (32) | |
| Stage ( [symbol] ) | | | | | | |
| IA & [symbol] | 5 | 2 | (40) | [symbol] | (60) | 0.170 |
| IIA & [symbol] | 82 | 57 | (70) | 25 | (30) | |
| Grade | | | | | | |
| (Differentation) | | | | | | |
| I (Well) | 16 | 16 | (100) | 0 | (0) | 0.007 |
| II (Moderate) | 59 | 37 | (63) | 22 | (37) | |
| III (Poor) | 12 | 6 | (50) | 6 | (50) | |
| Margin involvement | | | | | | |
| Free | 70 | 49 | (70) | 21 | (30) | 0.397 |
| [symbol] nvolved | 17 | 10 | (59) | 7 | (41) | |
| Lymphvascular | | | | | | |
| invasion | | | | | | |
| Negative | 27 | 20 | (74) | 7 | (26) | 0.402 |
| Positive | 60 | 39 | (65) | 21 | (35) | |
| [symbol] invasion | | | | | | |
| Negative | 8 | 6 | (75) | 2 | (25) | 0.648 |
| Positive | 79 | 53 | (67) | [symbol] 6 | (33) | |

Pearson’s Chi-square (x²) test was used to analyze the correlations. [symbol]
indicates data missing or illegible when filed

TABLE 5
Correlation between P-Y447 expression in cancer
cells and tumor formulation in patient-derived
xenografts in 44 pancreatic cancer cases.

| | | Patient-derived xenograft | | | | |
|---|---|---|---|---|---|---|
| | | | | Tumor | | |
| | Total | None | | formation | | |
| IHC staining | N | n (%) | | n (%) | | P value |
| | 44 | 26 | (59) | 18 | (41) | |
| Phospho-IL-17RB (P-Y447) | | | | | | |
| Low | 26 | 21 | (81) | 5 | (19) | 0.001 |
| High | 18 | 5 | (28) | 13 | (72) | |

Among these 44 patients with resected pancreatic tumors for PDX (Table 5), high IL-17RB P-Y447 expression correlated with the worse post-operative progression indicated by the recurrence and/or metastasis (FIG. 2C). Furthermore, larger proportion of tumor specimens with high P-Y447 was obtained from liver metastases than from primary tumors ( 12/17 vs 28/87, P=0.005, FIG. 10B). Taking together these results suggests that P-Y447 of IL-17RB is associated with more clinically aggressive pancreatic tumors.

### 2.3 MLK4 Is Identified for IL-17RB Y447 Phosphorylation and Essential for Downstream Oncogenic Signaling

To identify kinase phosphorylating Y-447 of IL-17RB, we performed co-immunoprecipitation followed by MS/MS analysis to identify proteins associated with IL-17RB upon IL-17B stimuli (FIG. 3A). There were 126 proteins co-immunoprecipitated with IL-17RB following IL-17B stimulation. The top three proteins that possess kinase activity include AP2-associated protein kinase 1 (AAK1, Accession (Uniprot ID): AAK1_HUMAN), homeodomain-interacting protein kinase 1 (HIPK1, Accession (Uniprot ID): HPK3_HUMAN), and mixed-lineage kinase 4 (MLK4, also known as KIAA1804 and MAP3K21, Accession (Uniprot ID): M3LK4 HUMAN). Upon IL-17B treatment, these three proteins indeed interacted with IL-17RB in a dose-dependent manner (FIG. 3B). Interestingly, only depletion of MLK4 (FIG. 3C), but not AAK1 (FIG. 11A) or HIPK1 (FIG. 11B), attenuated ERK1/2 phosphorylation induced by IL-17B. Moreover, based on reciprocal co-immunoprecipitation assay (FIG. 3D) and proximity ligation assay (PLA) (FIG. 3E), IL-17RB and MLK4 directly interacted upon IL-17B addition in vivo. Since MLK4 is a relatively poorly characterized kinase (19), we tested whether MLK4 is involved in oncogenic signaling by depleting MLK4 expression in both pancreatic and breast cancer cells. It was noticed that IL-17B-induced ERK1/2 phosphorylation (FIG. 12A), cytokine gene expression (FIG. 3F and FIG. 12B), colony formation (FIG. 3G and FIG. 12C), and cell invasion activity (FIG. 12D) were all abrogated in these MLK4-knockdown cancer cells, suggesting that MLK4 is essential for the IL-17RB-mediated oncogenic pathway.

### 2.4 IL-17RB Homo-Dimerization Is Required for Recruiting MLK4 for IL-17B-Induced Oncogenic Signaling

It was reported that IL-17RA heterodimerizes upon specific ligand binding to initiate intracellular signaling (20, 21). Although IL-17RB forms heterodimers with IL-17RA for IL-17E binding, it is unclear as to whether IL-17RB binds to another receptor chain for IL-17B binding. From the list of IL-17RB-interacting proteins induced by IL-17B (not shown), no other member of the IL-17 receptor family was found, implicating that IL-17RB may homo-dimerize following IL-17B binding. To test this possibility, we co-transfected HA- and Flag-tagged IL-17RB (IL-17RB-HA and IL-17RB-Flag, respectively) into IL-17RB-KO cells for co-IP experiments after IL-17B addition. Consistent with increased MLK4 binding and ERK1/2 phosphorylation, IL-17RB homo-dimerization was induced by IL-17B in a time-dependent manner (FIG. 4A). To confirm this finding, we expressed IL-17RB wild-type and mutant (IL-17RB^{FNmut}-Flag), in which the putative dimerization domain (fibronectin-like domain 2) (22) was truncated (FIG. 13A), in a compatible amount distributing on cell surface (FIG. 13B and FIG. 13C) for co-IP experiment following IL-17B addition. Unlike wild-type IL-17RB, IL-17RB^{FNmut} failed to form homodimer following stimulation (FIG. 4B). Similar results were obtained using Duolink in situ interaction assay visualizing the specific interaction of homodimers of IL-17RB on the cell membrane under either with ectopically expressed receptors (FIG. 4C and FIG. 13D) or with the endogenous condition (FIG. 4D). Moreover, abrogation of IL-17RB dimerization not only inhibited MLK4 binding (FIG. 4E), but also abolished IL-17B-induced phosphorylation of IL-17RB Y-447 and ERK1/2 (FIG. 4F). These results suggest that IL-17B triggered homo-dimerization of IL-17RB for MLK4 binding and downstream signal transmission.

IL-17RB has two different ligands: IL-17E and IL-17B (23, 24). Unlike IL-17B, IL-17E binds to IL-17RA/IL-17RB hetero-dimer to activate Th2 immune responses (24-26). To validate the specificity of IL-17RB dimerization in response to IL-17B, we performed co-IP experiments using IL-17RB-KO cells ectopically expressing HA-tagged and Flag-tagged IL-17RB, and His-tagged IL-17RA (FIG. 4G). We found that IL-17B specifically induced IL-17RB homo-dimerization, but not IL-17RA/IL-17RB hetero-dimerization that resulted from IL-17E binding (FIG. 4G). Moreover, IL-17B, but not IL-17E, induced MLK4 binding to IL-17RB, but to IL-17RB^{FNmut} (FIGS. 4E and 4G). Consistently, cells expressing IL-17RB^{FNmut} abrogated IL-17B-induced cytokines expression (FIG. 4H and FIG. 13E) and colony formation ability (FIG. 4I and FIG. 13F). Furthermore, homodimerization of IL-17RB induced by IL-17B was not affected by depletion of MLK4 or mutation of Y447F (FIG. 14A and FIG. 14B), indicating the dimerization of IL-17RB was prerequisite of the downstream signaling events. Taken together, these results suggest that IL-17RB/B signaling cascade is distinct from the other IL-17 receptor family members.

### 2.5 MLK4 Specifically Phosphorylates IL-17RB at Y447 Through Binding to Flexible Loop of IL-17RB.

MLK4 belongs to the mixed-lineage kinase family, whose members possess both serine/threonine and tyrosine kinase domains (19, 27). Although MLKs are known to have functional serine/threonine kinase activity (28, 29), their tyrosine kinase activity is rarely displayed. Since both phosphorylation of IL-17RB (FIG. 1 and FIG. 13G) and MLK4 binding to IL-17RB were observed (FIG. 4E and FIG. 4G) upon IL-17B stimuli, but not with IL-17E, IL-17RB is thought to be a substrate of MLK4. As shown in FIG. 5A, either knockdown (left) or knockout (right) of MLK4 nearly completely abolished IL-17RB phosphorylation at Y447 in both pancreatic and breast cancer cells (FIG. 5A and FIGS. 15A-15D), suggesting that other cellular enzymes may not substitute MLK4 for this action.

Next, to explore the critical interaction region of IL-17RB essential for the MLK4 binding, we modeled the intracellular domain (ICD) of human IL-17RB based on the structure of mouse I1-17rb (3vbc) (FIG. 16A and FIG. 16B). This revealed that the ICD is composed of five a-helices, five β-sheets and a flexible loop (FIG. 16A), and that mouse Y444 (homologous to human Y447) is located on the surface of the ICD (FIG. 16B). We then constructed eight individual mutants by deleting core residues of the a-helix and flexible loop distributed on the surface of the IL-17RB ICD (FIG. 5B). The deleted IL-17RB mutants and Flag-MLK4 were ectopically co-expressed in 293T cells for reciprocal co-IP experiments. The AV403—S416 mutant (Del-3) alone abrogated IL-17B-induced binding of IL-17RB and MLK4 (FIG. 5C). To assess the biological activity of Del-3, IL-17RB-KO pancreatic cancer cells were ectopically expressed Del-3 or wild-type IL-17RB, and the cell lysates were assayed for MLK4 binding and Y447 phosphorylation. Del-3 failed to bind to MLK4 and phosphorylate Y447 (FIG. 5D). Cells expressing Del-3 not only diminished ERK1/2 phosphorylation (FIG. 5D) and CCL20 and TFF1 expression (FIG. 5E), but also reduced their invasion (FIG. 5F) and colony formation (FIG. 5G) abilities compared to control cells. These results clearly indicate that the flexible loop containing V403—S416 of IL-17RB is critical for MLK4 binding and downstream oncogenic signaling.

To further assess the biological significance of MLK4 in IL-17B/IL-17RB signaling in an in vitro culture system, we performed rescue experiments by ectopically expressing wild-type or known MLK4 mutants (SH3 mutant: del 100-108; kinase-dead mutant: E314K, Y330H) (30) in MLK4-KO cells. Ectopic expression of the wild-type MLK4, but not the mutants, restored IL-17B-induced phosphorylation on IL-17RB Y447 and ERK1/2 (FIG. 5H), as well as downstream gene expression (FIG. 5I). Furthermore, when we used CEP-1347 to treat the high IL-17RB-expressing breast and pancreatic cancer cells, we noted that the phosphorylation of both ERK1/2 and IL-17RB (FIG. 15A), cytokine gene expressions (FIG. 15B), colony formation (FIG. 15C), and invasion properties of the cancer cells were abrogated (FIG. 15D). These data further suggest that the kinase activity of MLK4 for phosphorylating Y447 of IL-17RB is essential for IL-17B/IL-17RB oncogenic signaling.

### 2.6 P-Y447 IL-17RB Recruits TRIM56 Ubiquitin Ligase to Add K63-Linked Ubiquitin Onto Lysine 470 of IL-17RB for Oncogenic Signaling

To reveal the next step of how P-Y447 IL-17RB conducts downstream oncogenic signaling, we performed co-IP and MS/MS analysis to identify the proteins specifically bound to P-Y447 of IL-17RB using IL-17RB-KO BxPC3 cells that ectopically express WT or Y447F of IL-17RB (FIG. 6A). 61 proteins were found to be associated with P-Y447 IL-17RB upon IL-17B stimulation. Among these, the ubiquitin E3 ligase TRIM56 (tripartite-motif 56) was of interest because it is known to interact with and perform K63-linked ubiquitination on STING for recruitment of TBK1 to transduce downstream signals (31), and thus may regulate IL-17RB in a similar manner. To test this possibility, we performed in vivo (FIG. 6B) and in vitro (FIG. 17A) binding assays and observed that TRIM56 specifically bound WT, but not the Y447F mutant or non-tyrosine phosphorylated IL-17RB, upon IL-17B stimulation. Moreover, both knockdown and knockout of TRIM56 abrogated IL-17B-induced ERK1/2 phosphorylation (FIG. 6C and FIG. 17B), cytokine gene expression (FIG. 17C), colony formation (FIG. 17D), and invasion (FIG. 17E) capacities in both breast and pancreatic cancer cells. These results suggested that TRIM56 plays an essential role in IL-17B/IL-17RB oncogenic signaling.

To identify the region of IL-17RB that binds to TRIM56, we ectopically co-expressed the eight IL-17RB mutants generated as shown in FIG. 5B and HA-TRIM56 in 293T cells for reciprocal co-IP experiments. The ΔV403˜S416 (Del-3), ΔN458˜V462 (Del-6), and ΔK470˜Q484 (Del-7) mutants failed to bind TRIM56 upon IL-17B stimuli (FIG. 6D). Del-3 failed to bind to TRIM56 as expected because this region was critical for MLK4 binding and essential for IL-17RB phosphorylation at Y447 (FIG. 5C). Since phosphorylation of Y447 is required for TRIM56 binding, the region (N458˜V462) near Y447 may be important for IL-17RB to interact with TRIM56. To test this possibility, we ectopically expressed WT and Del-6 in 293T cells for co-IP experiments and noticed that Del-6 not only failed to bind the endogenous TRIM56, but also failed to induce ERK1/2 phosphorylation (FIG. 17F).

It was noticed that IL-17RB ubiquitination induced by IL-17B was composed of K63-linked, but not K48-linked, poly-ubiquitin (FIG. 18A), and depletion of TRIM56 abrogated IL-17B-induced K63-linked ubiquitination of IL-17RB (FIG. 6E). To determine the ubiquitination site of IL-17RB, we substituted three lysine residues on the surface of the IL-17RB intracellular domain (FIG. 18B) with arginine to generate K333R, K454R and K470R mutants, separately. These mutants were ectopically expressed in the IL-17RB-KO cells. In comparison to WT, which was poly-ubiquitinated upon IL-17B binding, the K470R, but not the K333R or K454R mutants, failed to be ubiquitinated (FIG. 6F and FIG. 18C), suggesting that K470 was the ubiquitination site. This finding may partly explain the observation that the deletion of K470˜Q484 of IL-17RB compromised TRIM56 binding (FIG. 6D and FIG. 18F). Furthermore, purified TRIM56 could ubiquitinate IL-17RB at K470 in vitro (FIG. 18D). Taken together, these results suggest that IL-17RB undergoes K63-linked poly-ubiquitination at K470 by TRIM56 upon IL-17B induction for oncogenic signaling transmission.

Transmission of the IL-17RA signaling includes ligand-induced oligomerization of IL-17RA, recruitment of ACT1 through the cytoplasmic SEFIR domain, and activation of the signal axis of ACT1/TRAF6/TAK1/TBK1 complexes (20, 32). Of note, it was observed that WT, but not K470R-mutant, IL-17RB interacted with ACT1, TRAF6 and TAK1 upon IL-17B induction (FIG. 6G), but knockdown of ACT1 or TRAF6, which has E3 ligase activity, did not affect IL-17B-induced IL-17RB ubiquitination (FIG. 18E). Consistently, ectopic expression of the K470R mutant, but not of the WT, in IL-17RB-KO cells failed to restore transduction of oncogenic signaling (FIG. 6H to FIG. 6J), further indicating that poly-ubiquitination of IL-17RB at K470 by TRIM56 was critical for IL-17B/IL-17RB oncogenic signaling.

Together, these results revealed the pivotal role of Y447 phosphorylation of IL-17RB by MLK4, which led to the recruitment of TRIM56 to poly-ubiquitinate IL-17RB at K470 in this oncogenic signaling cascade.

### 2.7 Disruption of the Interaction Between IL-17RB and MLK4 by a Specific Peptide Impedes Oncogenic Signaling

This mechanistic understanding highlighted ample opportunity to target and potentially block oncogenic signaling. To explore this further, we focused on the first step of tyrosine phosphorylation of IL-17RB by MLK4. We considered it likely that a peptide containing the flexible loop of V403—V416 would competitively inhibit MLK4 binding and Y447 phosphorylation. Thus, we synthesized two peptides, a control peptide (TAT₄₈₋₅₇) and a loop peptide (TAT-IL-17RB₄₀₃₋₄₁₆) (FIG. 7A) and demonstrated that both peptides can get into cells (FIG. 19A). We then treated cells ectopically expressing both Flag-MLK4 and HA-IL-17RB with these peptides and performed co-IP assays. Treatment with the loop peptide, but not the control peptide, disrupted the interaction between IL-17RB and MLK4 (FIG. 7B), abolished tyrosine phosphorylation and ubiquitination of IL-17RB, and ERK1/2 phosphorylation induced by IL-17B (FIG. 7C). Moreover, pancreatic cancer cells expressing IL-17RB diminished cytokine gene expression (FIG. 7D and FIG. 19B), invasion (FIG. 7E and FIG. 19C) and colony formation (FIG. 7F and FIG. 19D) abilities when treated with the loop peptide, suggesting that the loop peptide had the potential in blocking oncogenic signaling.

To test the therapeutic potential of the loop peptide (TAT-IL-17RB₄₀₃₋₄₁₆), two pancreatic cancer mouse models were used. The first model involved spontaneous pancreatic tumors generated in pancreas-specific KrasG12D-knockin mice (LSL-Kras^{G12D/+}; p53^{+/-}; Ela-Cre^{ERT}; EKP mice) upon cerulein treatment (33-35). Two parallel treatment protocols were employed (FIG. 7G). First, we measured lung metastases after euthanizing the mice at day 56 following the treatment protocol (FIG. 7G, upper). Treating with the loop peptide, but not the control, diminished the expression of P-Y447 IL-17RB in pancreatic tissues (FIG. 7H), suppressed lung metastasis nodule formation (FIG. 7I) and reduced the recruitment of MDSC and M2 macrophage (f FIG. 20A and FIG. 20B). Similarly, following the second protocol for measuring the survival time of the treated mice (FIG. 7G, bottom), it was noticed that treating with the loop peptide significantly prolonged lifespan (FIG. 7J).

To explore the therapeutic potential of the loop peptide in human pancreatic cancer cells, we transplanted GFP/Luc-tagged CFPAC1 cells orthotopically into NOD-SCID mice and performed in vivo imaging to monitor pancreatic tumor growth and distant metastasis in livers and lungs by IVIS following the protocol as illustrated (FIG. 21A). The control and the loop peptides were injected into the peritoneal cavities of mice bearing pancreatic tumors seven days after orthotopic implantation. The expression of phospho-IL-17RB was inhibited in the pancreatic tumors (FIG. 21B). Following the course, we monitored tumor size by IVIS (FIG. 21C) and noticed that treating with the loop peptide significantly impeded tumor growth (FIG. 21D) and prolonged lifespan as shown by Kaplan-Meier survival analysis (FIG. 21E). In the other set of the experiments, we euthanized the mice at Day 34 for metastasis analysis by the IVIS system and noticed that treating with the loop peptide significantly suppressed distant metastases of the pancreatic tumors to liver and lung (FIG. 21F and FIG. 21G).

### 3. Discussion

IL-17 receptor family members are known for their proinflammatory functions and for promoting an inflammatory microenvironment for tumor progression (36). However, unlike other IL-17 receptors, overexpression of IL-17RB confers tumorigenic activity to pancreatic and breast cancers. Mechanistically, IL-17RB forms a homodimer upon IL-17B binding, and recruits MLK4 to phosphorylate IL-17RB's Y447. The tyrosine-phosphorylated IL-17RB, in turn, recruits TRIM56 to add K63-linked ubiquitin chains onto IL-17RB's K470. Mutation of either Y447 or K470 of IL-17RB abrogated oncogenic signaling. The biological significance of this signaling mechanism was further demonstrated by blocking MLK4 binding to IL-17RB with a specific peptide containing amino acid sequence 403-416 of IL-17RB that leads to the loss of Y477 phosphorylation and K470 ubiquitination, thereby reducing tumorigenesis and metastasis, and prolonging the lifespan of pancreatic tumor-bearing mice.

It is known that IL-17RA serves as the common receptor that forms heterodimer with other IL-17 receptors. IL-17A and IL-17F exist either as homodimers or as a heterodimer, and all forms of the cytokine induce signals through an obligate dimeric IL-17RA and IL-17RC receptor complex (37). Similarly, IL-17E (IL-25) induces signals through IL-17RA and IL-17RB heterodimer to amplify Th2 immune responses. Unlike these IL-17 ligands that bind to IL-17RA heterodimers, IL-17B apparently binds to IL-17 RB homodimer (FIG. 4). A possibility exists that IL-17E may inhibit IL-17RB dimerization and phosphorylation if the level of IL-17RA is high. This was indeed the case in IL-17RA overexpressing cells as demonstrated (FIGS. 22A to 22D). Interestingly, IL-17RB was overexpressed in pancreatic cancer cells (7), but the expression of IL-17RA was barely or not detectable, conferring those cells a higher sensitivity to IL-17B than IL-17E response (FIGS. 22A to 22D). Moreover, the pancreatic cancer cells secreted IL-17B in an autocrine fashion to facilitate the activation of IL-17B/IL-17RB signaling for promoting cancer progression (7). These functional variation and differential distribution of the IL-17 cytokine and receptor family members may contribute to distinct biological function and disease pattern.

Importantly, the elucidated proximal mechanistic process of IL-17B/IL-17RB oncogenic signaling appears to be distinct among the IL-17 receptor family. IL-17B binds to IL-17RB and causes homo-dimerization, which is required for oncogenic signaling. Unlike RTK receptors, IL-17RB itself is not a kinase and recruits a mixed-lineage kinase. MLK-4, to phosphorylate Y447 of IL-17RB after homo-dimerization. This finding highlights IL-17RB's similarity to an RTK in that both are tyrosine-phosphorylated receptors. It was reported that the tyrosine kinase, Syk, may be involved in IL-17RA signaling (40). However, Syk has no influence on IL-17B-induced phosphorylation of IL-17RB and ERK1/2 (FIG. 11C), suggest Syk may have little or no role in IL-17RB oncogenic signaling. Thus, the tyrosine phosphorylation of IL-17RB by MLK4 to initiate IL-17B/IL-17RB oncogenic signaling apparently is different from the other IL-17 family receptors. Whether other IL-17 heterodimer receptors are phosphorylated by other tyrosine kinase in responding to their cognate ligands remains to be addressed.

Apparently, IL-17RB Y447 phosphorylation is required for recruiting TRIM56 E3 ligase to ubiquitinate IL-17RB on K470 (FIG. 7). Although ubiquitination by TRAF6 or ACT1 E3 ligase is observed on other mediators in IL-17RA signaling pathways (20, 41), removal of ACT1 or TRAF6 does not affect IL-17RB K63-linked ubiquitination (FIG. 18E). The function of K63-linked poly-ubiquitination of IL-17RB is to recruit other factors including ACT1 and perhaps other mediators, which are responsible for transmitting distal oncogenic signaling. Interestingly, ACT1 also relies on its E3 ligase activity for downstream IL-17 signaling transduction, leading to activation of the nuclear factor KB (NF-KB) and mitogen-activated protein kinase (MAPK) pathways, as well as the CCAAT-enhancer-binding proteins (C/EBPs) pathway (42, 43). Together, these transcription factors drive transcriptional activation of IL-17 target genes. Interestingly, the IL-17B/IL-17RB oncogenic pathway appears to be involved in the NF-KB and MAPK pathways to activate downstream targeted cytokine genes, which promote oncogenesis and metastasis (6, 7). Thus, the IL-17B/IL-17RB proximal signaling mechanism appears to be distinct as previously foreseen from the 3D structure (14).

Based on the understanding of this proximal signaling mechanism, there are several nodes apparently critical for oncogenic signal transduction. Thus, blocking these nodes would likely be effective strategies to specifically inhibit tumor malignancy (FIG. 23). Inhibition of IL-17RB by neutralizing antibodies was reported (7). Similarly, antibody-mediated removal of IL-17B is conceivable because blocking IL-17A with antibodies is clinically beneficial to psoriasis (44, 45). It is known that IL-17RB also serves as the receptor for IL-17E, which promotes Th2-skewed inflammatory responses (25, 26). Since Th2 is critical for immune homeostasis and defenses to bacterial infection and Th2 cytokines are linked to tumor growth and metastasis through suppression of the anti-tumor immunity (46, 47), a prolonged therapeutic blockage of IL-17RB such as using anti-IL-17RB antibody might result in deleterious clinical side effects. However, inhibition of MLK4 and IL-17RB interaction specifically by TAT-IL-17RB₄₀₃₋₄₁₆ would block the IL-17RB-mediated oncogenic signaling, but not Th2 immune responses induced by IL-17E/IL-17RB (FIG. 24), implicating a minimal adverse effect to Th2 immunity.

It was noticed that TRIM56 is the E3 ligase that mediates IL-17RB K63-linked ubiquitination (FIGS. 6A to 6J). Apparently, this TRIM56-mediated IL-17RB K63-linked ubiquitination process is also essential for IL-17RB/B oncogenic signal. Based on the above-demonstrated principle, the interaction surface between TRIM56 and IL-17RB may also provide a useful target for blocking this pathway. Thus. our results described herein not only elucidate the molecular basis of proximal IL-17B/IL-17RB oncogenic signaling, but also provide a new strategy using the loop peptide or its derivatives for treating certain pancreatic, breast, and other malignancies driven by this pathway.

### REFERENCES AND NOTES

1. Y. Iwakura, H. Ishigame, S. Saijo, S. Nakae, Functional specialization of interleukin-17 family members. Immunity 34, 149-162 (2011).
2. S. Aggarwal, A. L. Gurney, IL-17: prototype member of an emerging cytokine family. J Leukoc Biol 71, 1-8 (2002).
3. M. Novatchkova, A. Leibbrandt, J. Werzowa, A. Neubuser, F. Eisenhaber, The STIR-domain superfamily in signal transduction, development and immunity. Trends Biochem Sci 28, 226-229 (2003).
4. V. Ramirez-Carrozzi, A. Sambandam, E. Luis, Z. Lin, S. Jeet, J. Lesch, J. Hackney, J. Kim, M. Zhou, J. Lai, Z. Modrusan, T. Sai, W. Lee, M. Xu, P. Caplazi, L. Diehl, J. de Voss, M. Balazs, L. Gonzalez, Jr., H. Singh, W. Ouyang, R. Pappu, IL-17C regulates the innate immune function of epithelial cells in an autocrine manner. Nat Immunol 12, 1159-1166 (2011).
5. Y Su, J. Huang, X. Zhao, H. Lu, W. Wang, X. O. Yang, Y. Shi, X. Wang, Y Lai, C. Dong, Interleukin-17 receptor D constitutes an alternative receptor for interleukin-17A important in psoriasis-like skin inflammation. Sci Immunol 4, (2019).
6. C. K. Huang, C. Y Yang, Y M. Jeng, C. L. Chen, H. H. Wu, Y C. Chang, C. Ma, W. H. Kuo, K. J. Chang, J. Y. Shew, W. H. Lee, Autocrine/paracrine mechanism of interleukin-17B receptor promotes breast tumorigenesis through NF-kappaB-mediated antiapoptotic pathway. Oncogene 33, 2968-2977 (2014).
7 H. H. Wu, W. W. Hwang-Verslues, W. H. Lee, C. K. Huang, P. C. Wei, C. L. Chen, J. Y Shew, E. Y Lee, Y M. Jeng, Y W. Tien, C. Ma, W. H. Lee, Targeting IL-17B-IL-17RB signaling with an anti-IL-17RB antibody blocks pancreatic cancer metastasis by silencing multiple chemokines. J Exp Med 212, 333-349 (2015).
8. A. Maitra, F. Shen, W. Hanel, K. Mossman, J. Tocker, D. Swart, S. L. Gaffen, Distinct functional motifs within the IL-17 receptor regulate signal transduction and target gene expression. Proc Natl Acad Sci U S A 104, 7506-7511 (2007).
9. R. M. Onishi, S. J. Park, W. Hanel, A. W. Ho, A. Maitra, S. L. Gaffen, SEF/IL-17R (SEFIR) is not enough: an extended SEFIR domain is required for il-17RA-mediated signal transduction. J Biol Chem 285, 32751-32759 (2010).
10. B. Zhang, C. Liu, W. Qian, Y. Han, X. Li, J. Deng, Structure of the unique SEFIR domain from human interleukin 17 receptor A reveals a composite ligand-binding site containing a conserved alpha-helix for Act1 binding and IL-17 signaling. Acta Crystallogr D Biol Crystallogr 70, 1476-1483 (2014).
11. A. V. Garg, M. Ahmed, A. N. Vallejo, A. Ma, S. L. Gaffen, The deubiquitinase A20 mediates feedback inhibition of interleukin-17 receptor signaling. Sci Signal 6, ra44 (2013).
12. A. V. Garg, S. L. Gaffen, IL-17 signaling and A20: a balancing act. Cell Cycle 12, 3459-3460 (2013).
13. S. Zhu, W. Pan, P. Shi, H. Gao, F. Zhao, X. Song, Y Liu, L. Zhao, X. Li, Y Shi, Y. Qian, Modulation of experimental autoimmune encephalomyelitis through TRAF3-mediated suppression of interleukin 17 receptor signaling. J Exp Med 207, 2647-2662 (2010).
14. B. Zhang, C. Liu, W. Qian, Y. Han, X. Li, J. Deng, Crystal structure of IL-17 receptor B SEFIR domain. J Immunol 190, 2320-2326 (2013).
15. F. McAllister, J. M. Bailey, J. Alsina, C. J. Nirschl, R. Sharma, H. Fan, Y Rattigan, J. C. Roeser, R. H. Lankapalli, H. Zhang, E. M. Jaffee, C. G. Drake, F. Housseau, A. Maitra, J. K. Kolls, C. L. Sears, D. M. Pardoll, S. D. Leach, Oncogenic Kras activates a hematopoietic-to-epithelial IL-17 signaling axis in preinvasive pancreatic neoplasia. Cancer Cell 25, 621-637 (2014).
16. S. Furuta, Y. M. Jeng, L. Zhou, L. Huang, I. Kuhn, M. J. Bissell, W. H. Lee, IL-25 causes apoptosis of IL-25R-expressing breast cancer cells without toxicity to nonmalignant cells. Sci Transl Med 3, 78ra31 (2011).
17. S. C. Huang, P. C. Wei, W. W. Hwang-Verslues, W. H. Kuo, Y. M. Jeng, C. M. Hu, J. Y Shew, C. S. Huang, K. J. Chang, E. Y Lee, W. H. Lee, TGF-betal secreted by Tregs in lymph nodes promotes breast cancer malignancy via up-regulation of IL-17RB. EMBO Mol Med 9, 1660-1680 (2017).
18. S. R. Hubbard, W. T. Miller, Receptor tyrosine kinases: mechanisms of activation and signaling, Curr Opin Cell Biol 19, 117-123 (2007).
19. S. M. Craige, M. M. Reif, S. Kant, Mixed-Lineage Protein kinases (MLKs) in inflammation, metabolism, and other disease states. Biochim Biophys Acta 1862, 1581-1586 (2016).
20. S. L. Gaffen, Structure and signalling in the IL-17 receptor family. Nat Rev Immunol 9, 556-567 (2009).
21. J. M. Kramer, L. Yi, F. Shen, A. Maitra, X. Jiao, T. Jin, S. L. Gaffen, Evidence for ligand-independent multimerization of the IL-17 receptor. J Immunol 176, 711-715 (2006).
22. J. M. Kramer, W. Hanel, F. Shen, N. Isik, J. P. Malone, A. Maitra, W. Sigurdson, D. Swart, J. Tocker, T. Jin, S. L. Gaffen, Cutting edge: identification of a pre-ligand assembly domain (PLAD) and ligand binding site in the IL-17 receptor. J Immunol 179, 6379-6383 (2007).
23. Y Shi, S. J. Ullrich, J. Zhang, K. Connolly, K. J. Grzegorzewski, M. C. Barber, W. Wang, K. Wathen, V. Hodge, C. L. Fisher, H. Olsen, S. M. Ruben, I. Knyazev, Y. H. Cho, V. Kao, K. A. Wilkinson, J. A. Carrell, R. Ebner, A novel cytokine receptor-ligand pair. Identification, molecular characterization, and in vivo immunomodulatory activity. J Biol Chem 275, 19167-19176 (2000).
24. E. Tian, J. R. Sawyer, D. A. Largaespada, N. A. Jenkins, N. G. Copeland, J. D. Shaughnessy, Jr., Evi27 encodes a novel membrane protein with homology to the IL17 receptor. Oncogene 19, 2098-2109 (2000).
25. V. Dolgachev, B. C. Petersen, A. L. Budelsky, A. A. Berlin, N. W. Lukacs, Pulmonary IL-17E (IL-25) production and IL-17RB+ myeloid cell-derived Th2 cytokine production are dependent upon stem cell factor-induced responses during chronic allergic pulmonary disease. J Immunol 183, 5705-5715 (2009).
26. E. A. Rickel, L. A. Siegel, B. R. Yoon, J. B. Rottman, D. G. Kugler, D. A. Swart, P. M. Anders, J. E. Tocker, M. R. Comeau, A. L. Budelsky, Identification of functional roles for both IL-17RB and IL-17RA in mediating IL-25-induced activities. J Immunol 181, 4299-4310 (2008).
27. A. Rana, B. Rana, R. Mishra, G. Sondarva, V. Rangasamy, S. Das, N. Viswakarma, A. Kanthasamy, Mixed Lineage Kinase-c-Jun N-Terminal Kinase Axis: A Potential Therapeutic Target in Cancer. Genes Cancer 4, 334-341 (2013).
28. M. Martini, M. Russo, S. Lamba, E. Vitiello, E. H. Crowley, F. Sassi, D. Romanelli, M. Frattini, A. Marchetti, A. Bardelli, Mixed lineage kinase MLK4 is activated in colorectal cancers where it synergistically cooperates with activated RAS signaling in driving tumorigenesis. Cancer Res 73, 1912-1921 (2013).
29. A. Rana, K. Gallo, P. Godowski, S. Hirai, S. Ohno, L. Zon, J. M. Kyriakis, J. Avruch, The mixed lineage kinase SPRK phosphorylates and activates the stress-activated protein kinase activator, SEK-1. J Biol Chem 271, 19025-19028 (1996).
30. A. A. Marusiak, N. L. Stephenson, H. Baik, E. W. Trotter, Y Li, K. Blyth, S. Mason, P. Chapman, L. A. Puto, J. A. Read, C. Brassington, H. K. Pollard, C. Phillips, I. Green, R. Overman, M. Collier, E. Testoni, C. J. Miller, T. Hunter, O. J. Sansom, J. Brognard, Recurrent MLK4 Loss-of-Function Mutations Suppress JNK Signaling to Promote Colon Tumorigenesis. Cancer Res 76, 724-735 (2016).
31. T. Tsuchida, J. Zou, T. Saitoh, H. Kumar, T. Abe, Y Matsuura, T. Kawai, S. Akira, The ubiquitin ligase TRIM56 regulates innate immune responses to intracellular double-stranded DNA. Immunity 33, 765-776 (2010).
32. Y. Qian, C. Liu, J. Hartupee, C. Z. Altuntas, M. F. Gulen, D. Jane-Wit, J. Xiao, Y. Lu, N. Giltiay, J. Liu, T. Kordula, Q. W. Zhang, B. Vallance, S. Swaidani, M. Aronica, V. K. Tuohy, T. Hamilton, X. Li, The adaptor Act1 is required for interleukin 17-dependent signaling associated with autoimmune and inflammatory disease. Nat Immunol 8, 247-256 (2007).
33. C. C. Hsieh, Y M. Shyr, W. Y. Liao, T. H. Chen, S. E. Wang, P. C. Lu, P. Y Lin, Y. B. Chen, W. Y. Mao, H. Y. Han, M. Hsiao, W. B. Yang, W. S. Li, Y. P. Sher, C. N. Shen, Elevation of beta-galactoside alpha2,6-sialyltransferase 1 in a fructoseresponsive manner promotes pancreatic cancer metastasis. Oncotarget 8, 7691-7709 (2017).
34. S. C. Tang, L. Baeyens, C. N. Shen, S. J. Peng, H. J. Chien, D. W. Scheel, C. E. Chamberlain, M. S. German, Human pancreatic neuro-insular network in health and fatty infiltration. Diabetologia 61, 168-181 (2018).
35. S. C. Tang, C. N. Shen, P. Y. Lin, S. J. Peng, H. J. Chien, Y. H. Chou, C. E. Chamberlain, P. J. Pasricha, Pancreatic neuro-insular network in young mice revealed by 3D panoramic histology. Diabetologia 61, 158-167 (2018).
36. G. Murugaiyan, B. Saha, Protumor vs antitumor functions of IL-17. J Immunol 183, 4169-4175 (2009).
37. N. Amatya, A. V. Garg, S. L. Gaffen, IL-17 Signaling: The Yin and the Yang. Trends Immunol 38, 310-322 (2017).
38. P. G. Fallon, S. J. Ballantyne, N. E. Mangan, J. L. Barlow, A. Dasvarma, D. R. Hewett, A. McIlgorm, H. E. John, A. N. McKenzie, Identification of an interleukin (IL)-25-dependent cell population that provides IL-4, IL-5, and IL-13 at the onset of helminth expulsion. J Exp Med 203, 1105-1116 (2006).
39. A. M. Owyang, C. Zaph, E. H. Wilson, K. J. Guild, T. McClanahan, H. R. Miller, D. J. Cua, M. Goldschmidt, C. A. Hunter, R. A. Kastelein, D. Artis, Interleukin 25 regulates type 2 cytokine-dependent immunity and limits chronic inflammation in the gastrointestinal tract. J Exp Med 203, 843-849 (2006).
40. N. L. Wu, D. Y. Huang, H. N. Tsou, Y. C. Lin, W. W. Lin, Syk mediates IL-17-induced CCL20 expression by targeting Act1-dependent K63-linked ubiquitination of TRAF6. J Invest Dermatol 135, 490-498 (2015).
41. Z. Rong, L. Cheng, Y Ren, Z. Li, Y Li, X. Li, H. Li, X. Y Fu, Z. Chang, Interleukin-17F signaling requires ubiquitination of interleukin-17 receptor via TRAF6. Cell Signal 19, 1514-1520 (2007).
42. M. J. Ruddy, G. C. Wong, X. K. Liu, H. Yamamoto, S. Kasayama, K. L. Kirkwood, S. L. Gaffen, Functional cooperation between interleukin-17 and tumor necrosis factor-alpha is mediated by CCAAT/enhancer-binding protein family members. J Biol Chem 279, 2559-2567 (2004).
43. Z. Yao, S. L. Painter, W. C. Fanslow, D. Ulrich, B. M. Macduff, M. K. Spriggs, R. J. Armitage, Human IL-17: a novel cytokine derived from T cells. J Immunol 155, 5483-5486 (1995).
44. C. E. Griffiths, K. Reich, M. Lebwohl, P. van de Kerkhof, C. Paul, A. Menter, G. S. Cameron, J. Erickson, L. Zhang, R. J. Secrest, S. Ball, D. K. Braun, O. O. Osuntokun, M. P. Heffernan, B. J. Nickoloff, K. Papp, Uncover, U.-. investigators, Comparison of ixekizumab with etanercept or placebo in moderate-to-severe psoriasis (UNCOVER-2 and UNCOVER-3): results from two phase 3 randomised trials. Lancet 386, 541-551 (2015).
45. P. J. Mease, I. B. McInnes, B. Kirkham, A. Kavanaugh, P. Rahman, D. van der Heijde, R. Landewe, P. Nash, L. Pricop, J. Yuan, H. B. Richards, S. Mpofu, F. S. Group, Secukinumab Inhibition of Interleukin-17A in Patients with Psoriatic Arthritis. N Engl J Med 373, 1329-1339 (2015).
46. A. Budhu, M. Forgues, Q. H. Ye, H. L. Jia, P. He, K. A. Zanetti, U. S. Kammula, Y. Chen, L. X. Qin, Z. Y. Tang, X. W. Wang, Prediction of venous metastases, recurrence, and prognosis in hepatocellular carcinoma based on a unique immune response signature of the liver microenvironment. Cancer Cell 10, 99-111 (2006).
47. R. F. Gabitass, N. E. Annels, D. D. Stocken, H. A. Pandha, G. W. Middleton, Elevated myeloid-derived suppressor cells in pancreatic, esophageal and gastric cancer are an independent prognostic factor and are associated with significant elevation of the Th2 cytokine interleukin-13. Cancer Immunol Immunother 60, 1419-1430 (2011).
48. E. L. Jackson, N. Willis, K. Mercer, R. T. Bronson, D. Crowley, R. Montoya, T. Jacks, D. A. Tuveson, Analysis of lung tumor initiation and progression using conditional expression of oncogenic K-ras. Genes Dev 15, 3243-3248 (2001).
49. S. Y. Wu, C. C. Hsieh, R. R. Wu, J. Susanto, T. T. Liu, C. R. Shen, Y Chen, C. C. Su, F. P. Chang, H. M. Chang, D. Tosh, C. N. Shen, Differentiation of pancreatic acinar cells to hepatocytes requires an intermediate cell type. Gastroenterology 138, 2519-2530 (2010).
50. C. M. Hu, S. C. Tien, P. K. Hsieh, Y M. Jeng, M. C. Chang, Y T. Chang, Y J. Chen, Y. J. Chen, E. Y. P. Lee, W. H. Lee, High Glucose Triggers Nucleotide Imbalance through O-GlcNAcylation of Key Enzymes and Induces KRAS Mutation in Pancreatic Cells. Cell Metab 29, 1334-1349 e1310 (2019).
51. C. K. Huang, P. H. Chang, W. H. Kuo, C. L. Chen, Y M. Jeng, K. J. Chang, J. Y. Shew, C. M. Hu, W. H. Lee, Adipocytes promote malignant growth of breast tumours with monocarboxylate transporter 2 expression via beta-hydroxybutyrate. Nat Commun 8, 14706 (2017).
52. P. C. Wei, Y H. Hsieh, M. I. Su, X. Jiang, P. H. Hsu, W. T. Lo, J. Y. Weng, Y M. Jeng, J. M. Wang, P. L. Chen, Y C. Chang, K. F. Lee, M. D. Tsai, J. Y Shew, W. H. Lee, Loss of the oxidative stress sensor NPGPx compromises GRP78 chaperone activity and induces systemic disease. Mol Cell 48, 747-759 (2012).

1. A method for inhibiting interleukin-17B (IL-17B)/interleukin-17 receptor B (IL-17RB) activation and/or treating a disease or disorder associated with IL-17B/IL-17RB activation comprising administering to a subject in need thereof an effective amount of an antagonist of IL-17RB, wherein the IL-17RB antagonist targets the interaction between IL-17RB and mixed-lineage kinase 4 (MLK4), Y447 phosphorylation and/or K470 ubiquitination.

2. The method of claim 1, wherein the IL-17RB antagonist is a peptide or a small molecule inhibiting the binding of MLK4 to IL-17RB.

3. The method of claim 1, wherein the IL-17RB antagonist does not inhibit interleukin-17E (IL-17E)/IL-17RB-mediated type 2 immunity in the subject.

4. The method of claim 1, wherein the IL-17RB antagonist is an IL-17RB inhibitory peptide comprising a first segment that comprises the amino acid sequence X₁CDX₂X₃CX₄X₅X₆EGX₇X₈X₉ as set forth in SEQ ID NO:10, wherein X₁ is valine (V), isoleucine (I), leucine (L), alanine (A), methionine (M), X₂ is glycine (G) or serine (S), X₃ is threonine (T) or alanine (A), X₄ is glycine (G), serine (S) or aspartic acid (D), X₅ is lysine (K), histidine (H) or asparagine (N), X₆ is serine (S), lysine (K) or asparagine (N), X₇ is serine (S) or glycine (G), X₈ is proline (P) or alanine (A), and X₉ is serine (S), cysteine (C), Threonine (T), arginine (R) or histidine (H).

5. The method of claim 4, wherein the first segment comprises X₁CDX₂X₃CGX₅X₆EGSX₈X₉ as set forth in SEQ ID NO: 11, wherein X₁ is valine (V), isoleucine (I) or leucine (L), X₂ is glycine (G) or serine (S), X₃ is threonine (T) or alanine (A), X₅ is lysine (K) or histidine (H), X₆ is serine (S), lysine (K) or asparagine (N), X₈ is proline (P) or alanine (A), and X₉ is serine (S), cysteine (C), Threonine (T), arginine (R) or histidine (H).

6. The method of claim 4, wherein the first segment comprises X₁CDGTCGKSEGSPX₉ as set forth in SEQ ID NO: 12, wherein X₁ is valine (V) or isoleucine (I), and X₉ is serine (S), cysteine (C) or histidine (H).

7. The method of claim 4, wherein the first segment comprises VCDGTCGKSEGSPX₉ as set forth in SEQ ID NO: 13, wherein X₉ is serine (S) or histidine (H).

8. The method of claim 2, wherein the peptide has a length of less than 100 amino acids.

9. The method of claim 4, wherein the first segment comprises the amino acid sequence selected from the group consisting of
(SEQ ID NO: 14)
VCDGTCGKSEGSPS,
(SEQ ID NO: 15)
ICDGTCGKSEGSPC,
(SEQ ID NO: 16)
LCDSACGHKEGSAT,
(SEQ ID NO: 17)
LCDSACGHNEGSAR,
(SEQ ID NO: 18)
VCDGTCGKSEGSPH,
(SEQ ID NO: 19)
ACDGTCSNSEGGPH,
and
(SEQ ID NO: 20)
MCDSTCDKSEGSPH.

10. The method of claim 4, wherein the first segment is fused to a second segment that comprises a cell-penetrating peptide sequence.

11. The method of claim 10, wherein the cell-penetrating peptide sequence is selected from the group consisting of
(SEQ ID NO: 21)
RKKRRQRRR,
(SEQ ID NO: 22)
RQIKIWFQNRRMKWKK,
(SEQ ID NO: 23)
VRLPPPVRLPPPVRLPPP,
(SEQ ID NO: 24)
TRQARRNRRRWRERQR,
(SEQ ID NO: 25)
RRRNRTRRNRRRVR,
(SEQ ID NO: 26)
TRRQRTRRARRNR,
(SEQ ID NO: 27)
KRPAAIKKAGQAKKKK,
(SEQ ID NO: 28)
GWTLNSAGYLLGKINLKALAALAKKIL,
and
(SEQ ID NO: 29)
LLIILRRRIRKQAHAHSK.

12. The method of claim 11, wherein the IL-17RB inhibitory peptide comprises or consists of the amino acid sequence as set forth in RKKRRQRRRVCDGTCGKSEGSPS (SEQ ID NO: 30).

13. The method of claim 4, wherein the IL-17RB inhibitory peptide is a cyclic peptide.

14. The method of claim 1, wherein the disease or disorder is IL-17B/IL-17RB-mediated proliferation disorder.

15. The method of claim 14, wherein the disease or disorder is a cancer and a metastasis thereof.

16. The method of claim 15, wherein the cancer is selected from the group consisting of lung cancer, pancreatic cancer, breast cancer, colorectal cancer, liver cancer, kidney cancer, head and neck cancer, esophageal cancer, gastric cancer, biliary tract cancer, gallbladder and bile duct cancer, mammary cancer, ovarian cancer, cervical cancer, uterine body cancer, bladder cancer, prostate cancer, testicular tumor, osteogenic and soft-tissue sarcomas, leukemia, malignant lymphoma, multiple myeloma, skin cancer, brain tumor and pleural malignant mesothelioma.

17. The method of claim 16, wherein the cancer is breast cancer.

18. The method of claim 16, wherein the cancer is pancreatic cancer.

19. An IL-17RB inhibitory peptide that inhibits the binding of MLK4 to IL-17RB, wherein the IL-17RB inhibitory peptide comprises a first segment that comprises the amino acid sequence X₁CDX₂X₃CX₄X₅X₆EGX₇X₈X₉ as set forth in SEQ ID NO:10, wherein X₁ i is valine (V), isoleucine (I), leucine (L), alanine (A), methionine (M), X₂ is glycine (G) or serine (S), X₃ is threonine (T) or alanine (A), X₄ is glycine (G), serine (S) or aspartic acid (D), X₅ is lysine (K), histidine (H) or asparagine (N), X₆ is serine (S), lysine (K) or asparagine (N), X₇ is serine (S) or glycine (G), X₈ is proline (P) or alanine (A), and X₉ is serine (S), cysteine (C), Threonine (T), arginine (R) or histidine (H).

20. A recombinant nucleic acid comprising a nucleotide sequence encoding the IL-17RB inhibitory peptide that inhibits the binding of MLK4 to IL-17RB as defined in claim 19.

21. The recombinant nucleic acid of claim 20, which is a vector.

22. A composition, comprising the IL-17RB inhibitory peptide that inhibits the binding of MLK4 to IL-17RB as defined in claim 19 or a recombinant nucleic acid that comprises the IL-17RB inhibitory peptide, and a physiologically acceptable carrier.

23. The composition of claim 22, which is a pharmaceutical composition.

24.-35. (canceled)

36. A method for predicting the prognosis of cancer comprising measuring an expression level of phosphorylated IL-17RB in a sample obtained from a cancer patient and determining the prognosis of cancer in the patient based on the expression level of phosphorylated IL-17RB in the sample, wherein an elevated level of phosphorylated IL-17RB in the sample indicates poor prognosis.

37. A method for monitoring progression of cancer in a cancer patient, comprising
(a) measuring a level of phosphorylated IL-17RB protein in a first biological sample obtained from the patient at a first time-point;
(b) measuring a level of phosphorylated IL-17RB protein in a second biological sample obtained from the patient at a second time-point; and
(c) determining cancer progression in the patient based on the levels in the first and second biological samples wherein an elevated level of phosphorylated IL-17RB protein in the second biological sample as compared to that in the first biological sample is indicative of cancer progression.

38. The method of claim 36 or 37, wherein the cancer is pancreatic cancer.
